SEQUENCE LISTING (15 sequences)

SEQ ID NO: 1 - DNA, 16 bp, Artificial Sequence
  Description of Artifical Sequence Synthetic oligonucleotide
ctcaatccatggcagc

SEQ ID NO: 2 - DNA, 21 bp, Artificial Sequence
  Description of Combined DNA/RNA Molecule Synthetic oligonucleotide
gcaugcggccucuguuugatt

SEQ ID NO: 3 - DNA, 21 bp, Artificial Sequence
  Description of Combined DNA/RNA Molecule Synthetic oligonucleotide
ucaaacagaggccgcaugctt

SEQ ID NO: 4 - DNA, 19 bp, Artificial Sequence
  Description of Artifical Sequence Synthetic oligonucleotide
tctcccagcgtgcgcccat

SEQ ID NO: 5 - DNA, 16 bp, Artificial Sequence
  Description of Artifical Sequence Synthetic oligonucleotide
tggcaagcatcctgta

SEQ ID NO: 6 - DNA, 16 bp, Artificial Sequence
  Description of Artifical Sequence Synthetic oligonucleotide
tagcctgtcacttctc

SEQ ID NO: 7 - DNA, 17 bp, Artificial Sequence
  Description of Artifical Sequence Synthetic oligonucleotide
tagcttgtcccattctc

SEQ ID NO: 8 - protein, 12 aa, Artificial Sequence
  Description of Artifical Sequence Synthetic peptide
CYGRKKRRQRRR

SEQ ID NO: 9 - protein, 10 aa, Artificial Sequence
  Description of Artifical Sequence Synthetic peptide
CRRRRRRRRR

SEQ ID NO: 10 - protein, 13 aa, Artificial Sequence
  Description of Artifical Sequence Synthetic peptide
CYGRKKRRQRRRC

SEQ ID NO: 11 - protein, 23 aa, Artificial Sequence
  Description of Artifical Sequence Synthetic peptide
RRRQRRKKRGYRRRQRRKKRGYC

SEQ ID NO: 12 - protein, 4 aa, Artificial Sequence
  Description of Artifical Sequence Synthetic peptide
RGDC

SEQ ID NO: 13 - protein, 5 aa, Artificial Sequence
  Description of Artifical Sequence Synthetic peptide
RGDFC

SEQ ID NO: 14 - protein, 19 aa, Artificial Sequence
  Description of Artifical Sequence Synthetic peptide
SDGRGGGRRRQRRKKRGYC

SEQ ID NO: 15 - protein, 9 aa, Artificial Sequence
  Description of Artifical Sequence Synthetic peptide
RRRRRRRRR

(19) United States
(12) Patent Application Publication
(10) Pub. No.: US 2011/0305769 A1
(43) Pub. Date: Dec. 15, 2011

(54) BRANCHED CATIONIC LIPIDS FOR NUCLEIC ACIDS DELIVERY SYSTEM

(75) Inventors: Hong Zhao, Edison, NJ (US); Weili Yan, Piscataway, NJ (US); Lianjun Shi, Bridgewater, NJ (US); Dechun Wu, Bridgewater, NJ (US)

(73) Assignee: ENZON PHARMACEUTICALS, INC., Bridgewater, NJ (US)

(21) Appl. No.: 13/129,495

(22) PCT Filed: Jul. 31, 2009
(86) PCT No.: PCT/US2009/052462

§ 371 Date: Aug. 30, 2011

Related U.S. Application Data
(60) Provisional application No. 61/115,307, filed on Nov. 17, 2008.

(51) Int. Cl. A61K 9/14; A61K 31/7088; A61K 31/713; C12N 5/071; A61P 35/00; A61P 35/04; A61P 31/12; A61P 29/00; C07J 9/00; C12N 5/09; B82Y 5/00

(52) U.S. Cl. 424/499; 552/544; 552/506; 514/785; 514/788; 514/44 R; 514/44 A; 435/366; 435/375; 977/773; 977/797; 977/906; 977/800

(57) ABSTRACT

The present invention is directed to cationic lipid for the delivery of oligonucleotides and methods of modulating an expression of a targeted gene using the nanoparticle compositions. In particular, the invention relates to cholesterol and its derivatives having multiple positively charged moieties via branching spacers, and nanoparticle compositions of oligonucleotides encapsulated in a mixture of a cationic lipid, a fusogenic lipid and a PEG lipid.

### CROSS-REFERENCE TO RELATED APPLICATION

This application claims the benefit of priority from U.S. Provisional Patent Application Ser. No. 61/115,307 filed Nov. 17, 2008, the contents of which are incorporated herein by reference.

### FIELD OF THE INVENTION

The present invention relates to cationic lipids and nanoparticle compositions containing the same for the delivery of oligonucleotides and methods of modulating gene expression using nanoparticle compositions.

### BACKGROUND OF THE INVENTION

Therapy using nucleic acids has been proposed as an endeavor to treat various diseases over the past years. Therapy such as antisense therapy is a powerful tool in the treatment of disease because a therapeutic gene can selectively modulate gene expression associated with disease and minimize side effects which occur when other therapeutic approaches are used.

Therapy using nucleic acids has, however, been limited due to poor stability of genes and ineffective delivery. Several gene delivery systems have been proposed to overcome the hurdles and effectively introduce therapeutic genes into a targeted area, such as cancer cells or tissues in vitro and in vivo. Such attempts to improve delivery and enhance cellular uptake of therapeutic genes are directed to utilizing liposomes.

Currently available liposomes do not effectively deliver oligonucleotides into the body, although some progress has been made in the delivery of plasmids. In the delivery of oligonucleotides, desirable delivery systems should include positive charges sufficient enough to neutralize the negative charges of oligonucleotides. Recently, coated cationic liposomal (CCL) and Stable Nucleic Acid-Lipid Particles (SNALP) formulations described by Stuart, D. D., et al Biochim. Biophys. Acta, 2000, 1463:219-229 and Semple, S. C., et al, Biochim. Biophys. Acta, 2001, 1510:152-166, respectively, were reported to provide nanoparticles with small sizes, high nucleic acid encapsulation rate, good serum stability, and long circulation time. However, they did not show significantly improved in vivo activities especially in organs other than the liver, as compared to the use of the naked oligonucleotides.

It is desirable to provide a nucleic acids delivery system which allows enhanced cellular uptake and increased bioavailability of oligonucleotides in the cells, e.g. cancer cells. It is also desirable if the nucleic acids delivery system is stable for storage and safe for clinical use.

In spite of the attempts and advances, there continues to be a need to provide improved nucleic acids delivery systems. The present invention addresses this need.

### SUMMARY OF THE INVENTION

The present invention provides cationic lipids and nanoparticle compositions containing the same for nucleic acids delivery. Polynucleic acids, such as oligonucleotides, are encapsulated within nanoparticle complexes containing a mixture of a cationic lipid, a fusogenic lipid and a PEG lipid.

In accordance with this aspect of the invention, the cationic lipids for the delivery of nucleic acids (i.e., an oligonucleotide) have Formula (I):

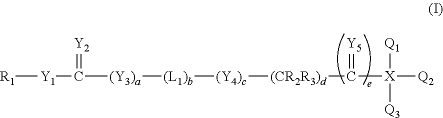

wherein
R₁ is a cholesterol or analog thereof;
Y₁, Y₂ and Y₅ are independently O, S or NR₄;
Y₃ and Y₄ are independently O, S or NR₅;
L₁ is a spacer having a substituted saturated or unsaturated, branched or linear, C₃₋₅₀ alkyl, wherein one or more carbons are replaced with NR₆, O, S or C(═Y), wherein Y is O, S or NR₄;
(a), (c) and (e) are independently 0 or 1;
(b) is 0 or a positive integer;
(d) is 0 or a positive integer;
X is C or P;
Q₁ is H, C₁₋₆ alkyl, NH₂, or -(L₁₁)_{d1}-R₁₁;
Q₂ is H, C₁₋₆ alkyl, NH₂, or -(L₁₂)_{d2}-R₁₂;
Q₃ is (═O), H, C₁₋₆ alkyl, NH₂, or -(L₁₃)_{d3}-R₁₃,
   provided that
   (i) when X is C, Q₃ is not (═O); and
   (ii) when X is P, (e) is 0,
      wherein
      L₁₁, L₁₂ and L₁₃ are independently selected bifunctional spacers;
      (d1), (d2) and (d3) are independently 0 or a positive integer;
      R₁₁, R₁₂ and R₁₃ are independently hydrogen, NH₂,

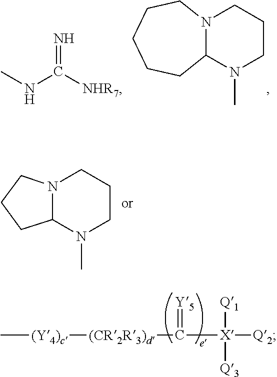

wherein
Y′₄ is O, S, or NR′₅;
Y′₅ are independently O, S or NR′₄;
(c′) and (e′) are independently 0 or 1;
(d′) is 0 or a positive integer;
X′ is C or P;
Q′₁ is H, C₁₋₆ alkyl, NH₂, or -(L′₁₁)_{d′1}-R′₁₁;
Q′₂ is H, C₁₋₆ alkyl, NH₂, or -(L′₁₂)_{d′2}-R′₁₂;
Q′₃ is a lone electron pair, (═O), H, C₁₋₆ alkyl, NH₂, or -(L′₁₃)_{d′3}-R′₁₃;
   provided that
   (i) when X′ is C, Q′₃ is not (═O); and
   (ii) when X′ is P, (e′) is 0,
   wherein
   L′₁₁, L′₁₂ and L′₁₃ are independently selected bifunctional spacers;
   (d′1), (d′2) and (d′3) are independently 0 or a positive integer;
   R′₁₁, R′₁₂ and R′₁₃ are independently hydrogen, NH₂,

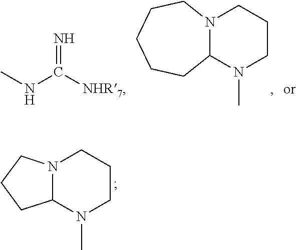

and
R₂₋₇, R′₂₋₅ and R′₇ are independently selected from among hydrogen, amino, substituted amino, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, and substituted C₁₋₆ heteroalkyl,
provided that at least one or more of Q₁₋₃ and Q′₁₋₃ include

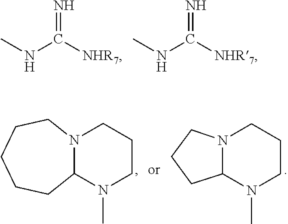

The present invention also provides nanoparticle compositions for nucleic acids delivery. Nucleic acids, such as oligonucleotides, are encapsulated within nanoparticle complexes containing a mixture of a cationic lipid, a fusogenic lipid and a PEG lipid.

In accordance with this aspect of the invention, the nanoparticle composition for the delivery of nucleic acids (i.e., an oligonucleotide) includes:

(i) a cationic lipid of Formula (I);

(ii) a fusogenic lipid; and

(iii) a PEG lipid.

The present invention further provides methods for the delivery of nucleic acids (preferably, oligonucleotides) to a cell or tissue, in vivo and in vitro. Oligonucleotides introduced by the methods described herein can modulate the expression of a target gene.

One aspect of the present invention provides methods of inhibiting expression of a target gene, i.e., oncogenes and genes associated with disease in mammals, preferably humans. The methods include contacting cells, such as cancer cells or tissues, with a nanoparticle/nanoparticle complex prepared from the nanoparticle composition described herein. The oligonucleotides encapsulated within the nanoparticle are released, which then mediate the down-regulation of mRNA or protein in the cells or tissues being treated. The treatment with the nanoparticle allows modulation of target gene expression (and the attendant benefits associated therewith) in the treatment of malignant disease, such as inhibition of the growth of cancer cells. Such therapies can be carried out as a single treatment or as part of a combination therapy, with one or more useful and/or approved treatments.

Further aspects include methods of making the cationic lipids of Formula (I) as well as nanoparticles containing the same.

The nanoparticles described herein have improved in vitro cellular uptake of LNA-containing oligonucleotides (LNA-ONs) in human cancer cells and enhanced the delivery of LNA-ONs to the tumors in mammals.

The cationic lipids described herein neutralize the negative charges of nucleic acids and facilitate cellular uptake of the nanoparticle containing the nucleic acids therein. The cationic lipids herein further provide multiple units of cationic moieties per cholesterol moiety, to provide higher efficiency in (i) neutralizing the negative charges of the nucleic acids and (ii) forming a tighter ionic complex with nucleic acids. This technology is advantageous for the delivery of therapeutic oligonucleotides and the treatment of mammals, i.e., humans, using therapeutic oligonucleotides including LNA, and those based on siRNA, microRNA, and MOE antisense.

Another advantage of the cationic lipids described herein is that they provide a means to control the size of the nanoparticles by forming multiple ionic complexes with nucleic acids.

The cationic lipids described herein stabilize nanoparticle complexes and nucleic acids therein in biological fluids. Without being bound by any theory, it is believed that the nanoparticle complex enhances the stability of the encapsulated nucleic acids, at least in part by shielding the molecules from nucleases, thereby protecting from degradation. The nanoparticles based on cationic lipids of Formula (I) described herein stabilize the encapsulated nucleic acids.

The cationic lipids described herein allow high efficiency (e.g. above 70%, preferably above 80%) of nucleic acids (oligonucleotides) loading compared to art-known neutral or negatively charged nanoparticles, which typically have loadings of about or less than 10%. Without being bound by any theory, the high loading is achieved in part by the fact that the guanidinium group having high pKa (13-14) of the cationic lipids of Formula (I) described herein forms substantially compact zwitter ionic hydrogen bonds with phosphate groups of nucleic acids, and thereby enabling more nucleic acids to be effectively packaged into the inner compartment of nanoparticles.

The nanoparticles described herein provide a further advantage over neutral or negatively charged nanoparticles, in that the aggregation or precipitation of nanoparticles is less likely to occur. Without being bound by any theory, the desired property is attributed in part to the fact that the cationic lipids forming hydrogen bonds or electrostatic interaction with nucleic acids are encapsulated within the nanoparticles, and noncationic/fasogenic lipids and PEG lipids surround the cationic lipid and nucleic acids.

The nanoparticles described herein provide another advantage, such as high transfection efficiency. The nanoparticles described herein allow transfection of cells in vitro and in vivo without the aid of a transfection agent. The nanoparticles are safe because they do not have the same toxicity as art-known nanoparticles which require transfection agents. The high transfection efficiency of the nanoparticles also provides a means to deliver therapeutic nucleic acids into a nucleus.

The nanoparticles described herein also provide an advantageous stability and flexibility in the preparation of the nanoparticles. The nanoparticles can be prepared in a wide pH range such as about 2-12. The nanoparticles described herein also can be used clinically at a desirable physiological pH, such as about 7.2-7.6.

The nanoparticle delivery systems described herein also allow sufficient amounts of the therapeutic oligonucleotides to be selectively available at the desired target area, such as cancer cells via EPR (Enhanced Permeation and Retention) effects. The nanoparticle composition described herein thus improves specific mRNA downregulation in cancer cells or tissues.

Another advantage is that the cationic lipids described herein allow for the preparation of homogenous nanoparticles in size and stability of the nanoparticles during storage. The nanoparticle complexes containing the cationic lipids described herein are stable under buffer conditions. This is a significant advantage over prior art technologies since this feature provides clinicians with reliable and flexible treatment regimens.

Another advantage is that the nanoparticles described herein allow delivery of one or more, same or different antisense target oligonucleotides, thereby attaining synergistic effects in treatment of disease.

It has been increasingly attractive to treat human diseases at the gene level. Oligonucleotides, including locked nucleic acids and siRNA, have the potential to prohibit unwanted gene expression. The present invention allows for enhancement in cellular uptake and accumulation of nucleic acids such as LNA-ONs in the target area, cells or tissues. In addition, the cationic lipid-based nanoparticles described herein are safe to deliver oligonucleotides in vivo to improve their pharmacokinetic profile, cell penetration, and specific tumor targeting, as compared to viral delivery systems.

Another advantage of the present invention is that the nanoparticles described herein enable potent down-modulation of target mRNA in human tumor cells without the aid of transfection agents and improves the cellular delivery of nucleic acids in tumor-bearing mammals.

Other and further advantages will be apparent from the following description.

For purposes of the present invention, the term “residue” shall be understood to mean that portion of a compound, to which it refers, e.g., cholesterol, etc. that remains after it has undergone a substitution reaction with another compound.

For purposes of the present invention, the term “alkyl” refers to a saturated aliphatic hydrocarbon, including straight-chain, branched-chain, and cyclic alkyl groups. The term “alkyl” also includes alkyl-thio-alkyl, alkoxyalkyl, cycloalkylalkyl, heterocycloalkyl, and C₁₋₆ alkylcarbonylalkyl groups. Preferably, the alkyl group has 1 to 12 carbons. More preferably, it is a lower alkyl of from about 1 to 7 carbons, yet more preferably about 1 to 4 carbons. The alkyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆hydrocarbonyl, aryl, and amino groups.

For purposes of the present invention, the term “substituted” refers to adding or replacing one or more atoms contained within a functional group or compound with one of the moieties from the group of halo, oxy, azido, nitro, cyano, alkyl, alkoxy, alkyl-thio, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ alkylcarbonylalkyl, aryl, and amino groups.

For purposes of the present invention, the term “alkenyl” refers to groups containing at least one carbon-carbon double bond, including straight-chain, branched-chain, and cyclic groups. Preferably, the alkenyl group has about 2 to 12 carbons. More preferably, it is a lower alkenyl of from about 2 to 7 carbons, yet more preferably about 2 to 4 carbons. The alkenyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “alkynyl” refers to groups containing at least one carbon-carbon triple bond, including straight-chain, branched-chain, and cyclic groups. Preferably, the alkynyl group has about 2 to 12 carbons. More preferably, it is a lower alkynyl of from about 2 to 7 carbons, yet more preferably about 2 to 4 carbons. The alkynyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups. Examples of “alkynyl” include propargyl, propyne, and 3-hexyne.

For purposes of the present invention, the term “aryl” refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include phenyl, naphthyl, 1,2,3,4-tetrahydronaphthalene and biphenyl. Preferred examples of aryl groups include phenyl and naphthyl.

For purposes of the present invention, the term “cycloalkyl” refers to a C₃₋₈ cyclic hydrocarbon. Examples of cycloalkyl include cyclopropyl, cyclobutyl, cyclopentyl, cyclohexyl, cycloheptyl and cyclooctyl.

For purposes of the present invention, the term “cycloalkenyl” refers to a C₃₋₈ cyclic hydrocarbon containing at least one carbon-carbon double bond. Examples of cycloalkenyl include cyclopentenyl, cyclopentadienyl, cyclohexenyl, 1,3-cyclohexadienyl, cycloheptenyl, cycloheptatrienyl, and cyclooctenyl.

For purposes of the present invention, the term “cycloalkylalkyl” refers to an alklyl group substituted with a C₃₋₈ cycloalkyl group. Examples of cycloalkylalkyl groups include cyclopropylmethyl and cyclopentylethyl.

For purposes of the present invention, the term “alkoxy” refers to an alkyl group of indicated number of carbon atoms attached to the parent molecular moiety through an oxygen bridge. Examples of alkoxy groups include methoxy, ethoxy, propoxy and isopropoxy.

For purposes of the present invention, an “alkylaryl” group refers to an aryl group substituted With an alkyl group.

For purposes of the present invention, an “aralkyl” group refers to an alkyl group substituted with an aryl group.

For purposes of the present invention, the term “alkoxyalkyl” group refers to an alkyl group substituted with an alkoxy group.

For purposes of the present invention, the term “alkyl-thio-alkyl” refers to an alkyl-S-alkyl thioether, for example methylthiomethyl or methylthioethyl.

For purposes of the present invention, the term “amino” refers to a nitrogen containing group, as is known in the art, derived from ammonia by the replacement of one or more hydrogen radicals by organic radicals. For example, the terms “acylamino” and “alkylamino” refer to specific N-substituted organic radicals with acyl and alkyl substituent groups respectively.

For purposes of the present invention, the term “alkylcarbonyl” refers to a carbonyl group substituted with alkyl group.

For purposes of the present invention, the term “halogen’ or “halo” refers to fluorine, chlorine, bromine, and iodine.

For purposes of the present invention, the term “heterocycloalkyl” refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include piperazine, morpholine, piperidine, tetrahydrofuran, pyrrolidine, and pyrazole. Preferred heterocycloalkyl groups include piperidinyl, piperazinyl, morpholinyl, and pyrrolidinyl.

For purposes of the present invention, the term “heteroaryl” refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, triazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

For purposes of the present invention, the term “heteroatom” refers to nitrogen, oxygen, and sulfur.

In some embodiments, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aryls include moieties such as naphthyl; substituted aryls include moieties such as 3-bromo phenyl; aralkyls include moieties such as tolyl; heteroalkyls include moieties such as ethylthiophene; substituted heteroalkyls include moieties such as 3-methoxy-thiophene; alkoxy includes moieties such as methoxy; and phenoxy includes moieties such as 3-nitrophenoxy. Halo shall be understood to include fluoro, chloro, iodo and bromo.

For purposes of the present invention, “positive integer” shall be understood to include an integer equal to or greater than 1 and as will be understood by those of ordinary skill to be within the realm of reasonableness by the artisan of ordinary skill.

For purposes of the present invention, the term “linked” shall be understood to include covalent (preferably) or noncovalent attachment of one group to another, i.e., as a result of a chemical reaction.

The terms “effective amounts” and “sufficient amounts” for purposes of the present invention shall mean an amount which achieves a desired effect or therapeutic effect, as is understood by those of ordinary skill in the art.

The term “nanoparticle” and/or “nanoparticle complex” formed using the nanoparticle composition described herein refers to a lipid-based nanocomplex. The nanoparticle contains nucleic acids such as oligonucleotides encapsulated in a mixture of a cationic lipid, a fusogenic lipid, and a PEG lipid. Alternatively, the nanoparticle can be formed without nucleic acids.

For purposes of the present invention, the term “therapeutic oligonucleotide” refers to an oligonucleotide used as a pharmaceutical or diagnostic.

For purposes of the present invention, “modulation of gene expression” shall be understood as broadly including down-regulation or up-regulation of any types of genes, preferably associated with cancer and inflammation, compared to a gene expression observed in the absence of the treatment with the nanoparticle described herein, regardless of the route of administration.

For purposes of the present invention, “inhibition of expression of a target gene” shall be understood to mean that mRNA expression or the amount of protein translated are reduced or attenuated when compared to that observed in the absence of the treatment with the nanoparticle described herein. Suitable assays of such inhibition include, e.g., examination of protein or mRNA levels using techniques known to those of ordinary skill in the art such as dot blots, northern blots, in situ hybridization, ELISA, immunoprecipitation, enzyme function, as well as phenotypic assays known to those of ordinary skill in the art. The treated conditions can be confirmed by, for example, decrease in mRNA levels in cells, preferably cancer cells or tissues.

Broadly speaking, successful inhibition or treatment shall be deemed to occur when the desired response is obtained. For example, successful inhibition or treatment can be defined by obtaining, e.g., 10% or higher (i.e., 20% 30%, 40%) downregulation of genes associated with tumor growth inhibition. Alternatively, successful treatment can be defined by obtaining at least 20%, preferably 30% or more preferably 40% or higher (i.e., 50% or 80%) decrease in oncogene mRNA levels in cancer cells or tissues, including other clinical markers contemplated by the artisan in the field, when compared to that observed in the absence of the treatment with the nanoparticle described herein.

Further, the use of singular terms for convenience in description is in no way intended to be so limiting. Thus, for example, reference to a composition comprising an oligonucleotide, a cholesterol analog, a cationic lipid, a fusogenic lipid, a PEG lipid, etc., refers to one or more molecules of that oligonucleotide, cholesterol analog, cationic lipid, fuosogenic lipid, PEG lipid, etc. It is also contemplated that the oligonucleotide can be of the same or different kind of gene. It is also to be understood that this invention is not limited to the particular configurations, process steps, and materials disclosed herein as such configurations, process steps, and materials may vary somewhat.

It is also to be understood that the terminology employed herein is used for the purpose of describing particular embodiments only and is not intended to be limiting, since the scope of the present invention will be limited by the appended claims and equivalents thereof.

### BRIEF DESCRIPTION OF THE DRAWINGS

FIG. 1 schematically illustrates a reaction scheme of preparing compound 6, as described in Examples 3-8.

FIG. 2 schematically illustrates a reaction scheme of preparing compound 11, as described in Examples 9-13.

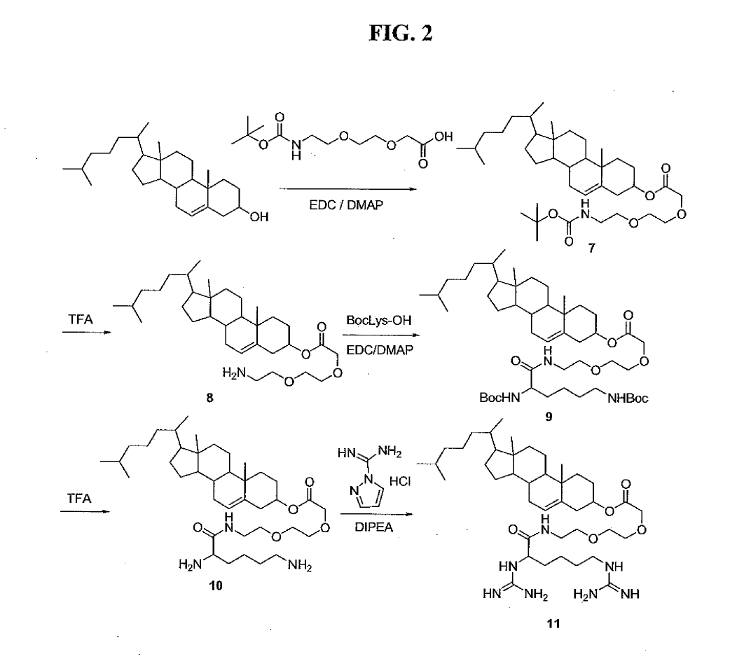

FIG. 3 schematically illustrates a reaction scheme of preparing compound 14, as described in Examples 14-16.

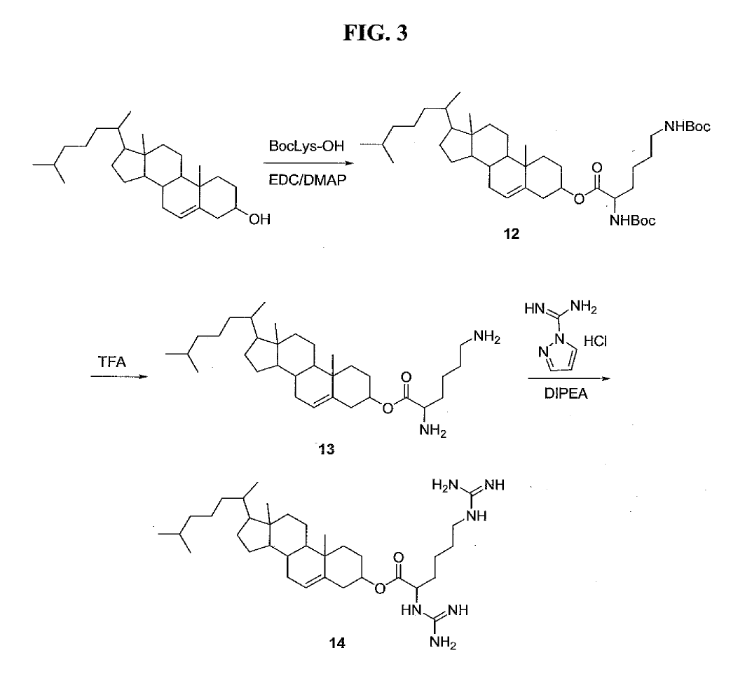

### DETAILED DESCRIPTION OF THE INVENTION

In one aspect of the present invention, there are provided cationic lipids containing multiple cationic moieties. In another aspect of the invention, there are provided nanoparticle compositions containing the same for the delivery of nucleic acids. The nanoparticle composition may contain (i) a cationic lipid of Formula (I); (ii) a fusogenic lipid; and (iii) a PEG lipid. The nucleic acids contemplated include oligonucleotides or plasmids, and preferably oligonucleotides. The nanoparticles prepared by using the nanoparticle composition described herein include nucleic acids encapsulated in the lipid carrier.

### A. Cationic Lipids of Formula (I)

### 1. Overview

The cationic lipids described herein have Formula (I):

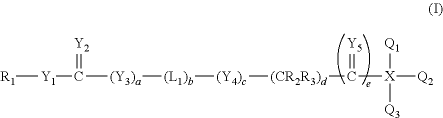

wherein
R₁ is a cholesterol or analog thereof;
Y₁, Y₂ and Y₅ are independently O, S or NR₄, preferably O;
Y₃ and Y₄ are independently O, S or NR₅, preferably O or NR₅;
L₁ is a spacer having a substituted saturated or unsaturated, branched or linear, C₃₋₅₀ alkyl (i.e., C₃₋₄₀ alkyl, C₃₋₂₀ alkyl, C₃₋₁₅ alkyl, C₃₋₁₀ alkyl, etc.), wherein one or more carbons are replaced with NR₆, O, S or C(═Y), preferably O, wherein Y is O, S or NR, preferably O;
(a), (c) and (e) are independently 0 or 1;
(b) is 0 or a positive integer, preferably 0 or a postive integer from about 1 to about 5 (e.g., 0, 1, 2, 3, 4, 5), and more preferably 0 or an integer from about 1 to about 3 (e.g., 0, 1, 2, 3), provided that when (b) is 0, both (a) and (c) are not simultaneously positive integers;
(d) is 0 or a positive integer, preferably 0 or a positve integer from about 1 to about 5 (e.g., 0, 1, 2, 3, 4, 5);
X is C or P;
Q₁ is H, C₁₋₆ alkyl, NH₂, or -(L₁₁)_{d1}-R₁₁;
Q₂ is H, C₁₋₆ alkyl, NH₂, or -(L₁₂)_{d2}-R₁₂;
Q₃ is (═O), H, C₁₋₆ alkyl, NH₂, or -(L₁₃)_{d3}-R₁₃,
   provided that
   (i) when X is C, Q₃ is not (═O); and
   (ii) when X is P, (e) is 0,
      wherein
      L₁₁, L₁₂ and L₁₃ are independently selected bifunctional spacers;
      (d1), (d2) and (d3) are independently 0 or a positive integer, preferably 0 or an integer from about 1 to about 9 (e.g., 1, 2, 3, 4, 5, 6), and more preferably 0 or a positive integer from about 1 to about 3 (e.g., 1, 2, 3, 4);
      R₁₁, R₁₂ and R₁₃ are independently hydrogen, NH₂,

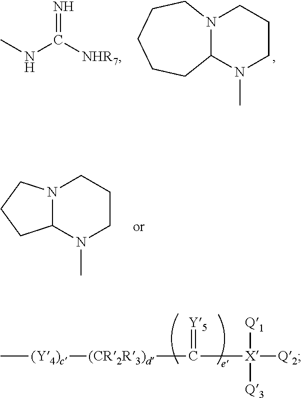

wherein
Y′₄ is O, S, or NR′₅, preferably O or NR′₅;
Y′₅ are independently O, S or NR′₄, preferably O;
(c′) and (e′) are independently 0 or 1;
(d′) is 0 or a positive integer; preferably 0 or a positive integer from about 1 to about 10 (e.g., 1, 2, 3, 4, 5, 6), and more preferably 0 or a positive integer from about 1 to about 4 (e.g., 1, 2, 3)
   X′ is C or P;
   Q′₁ is H, C₁₋₆ alkyl, NH₂, or -(L′₁₁)_{d′1}-R′₁₁;
      Q′₂ is H, C₁₋₆ alkyl, NH₂, or -(L′₁₂)_{d′2}-R′₁₂;
      Q′₃ is (═O), H, C₁₋₆ alkyl, NH₂, or -(L′₁₃)_{d′3}-R′₁₃,
         provided that
         (i) when X′ is C, Q′₃ is not (═O); and
         (ii) when X′ is P, (e′) is 0,
         Wherein
         L′₁₁, L′₁₂ and L′₁₃ are independently selected bifunctional spacers;
         (d′1), (d′2) and (d′3) are independently 0 or a positive integer, preferably 0, 1, 2, 3, 4;
         R′₁₁, R′₁₂ and R′₁₃ are independently hydrogen, NH₂,

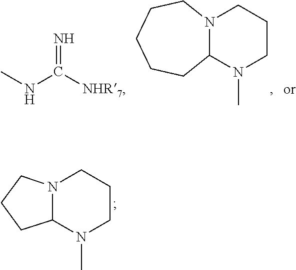

and
R₂₋₇, R′₂₋₅ and R′₇ are independently selected from among hydrogen, amino, substituted amino, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, and substituted C₁₋₆ heteroalkyl, preferably H, methyl, ethyl and propyl, and more preferably H,
provided that at least one or more (eg., one, two, three) of Q₁₋₃ and Q′₁₋₃ include

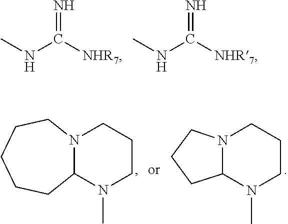

For purposes of the present invention, each L₁ is the same or different when (b) is equal to or greater than 2.

For purposes of the present invention, each L₁₁, L₁₂ and L₁₃ is the same or different when each (d1), (d2) and (d3), respectively, is equal to or greater than 2.

For purposes of the present inventions, each L′₁₁, L′₁₂ and L′₁₃ is the same or different when each (d′1), (d′2) and (d′3), respectively, is equal to or greater than 2.

In one preferred aspect, both (d1) and (d2) are not zero. In another preferred aspect, (d1), (d2), (d3), (d′1), (d′2) and (d′3) are not simultaneously zero.

In certain aspects of the invention, (a), (b), (c), (d) and (e) are all zero.

In one embodiment, the cationic lipids have Formula (Ia):

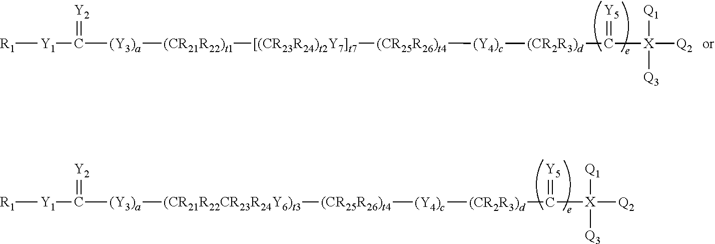

wherein

Y₆ and Y₇ are independently O, S or NR₂₉, preferably O or NH;

R₂₁₋₂₆ and R₂₉ are independently selected from among hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆alkoxy, phenoxy and C₁₋₆heteroalkoxy, preferably hydrogen, methyl, ethyl and propyl;

(t1), (t2), (t3), (t4), and (t7) are independently 0 or a positive integer, preferably from about 1 to about 10 (e.g., 1, 2, 3, 4, 5), and more preferably 1, 2, 3,
wherein R₂₁ and R₂₂ in each occurrence are independently the same or different, when (t1) is equal to or greater than 2;
wherein R₂₃, R₂₄, and Y₇ in each occurrence are independently the same or different, when (t2) and (t7) are indenpendently equal to or greater than 2,
wherein R₂₁, R₂₂, R₂₃, R₂₄, and Y₆, in each occurrence, are independently the same or different, when (t3) is equal to or greater than 2,
wherein R₂₅ and R₂₆ in each occurrence are independently the same or different, when (t4) is equal to or greater than 2; and

all the other variables are as defined above.

The cationic lipids of Formula (I) described herein would carry a net positive charge at a selected pH, such as pH<13 (e.g. pH 6-12, pH 6-8).

### 2. Spacer L₁

In one aspect of the invention, the spacer L₁ is a bifunctional linker having a substituted saturated or unsaturated, branched or linear, C₃₋₅₀ alkyl (i.e., C₃₋₄₀ alkyl, C₃₋₂₀ alkyl, C₃₋₁₅ alkyl, C₃₋₁₀ alkyl, etc.), wherein optionally one or more carbons are replaced with NR₆, O, S or C(═Y), (preferably O or NH), but not exceeding 70% (i.e., less than 60%, 50%, 40%, 30%, 20%, 10%) of the carbons being replaced.

Some illustrative examples of L₁, when combined with a moiety of (Y₄)_{c}—(CR₂R₃)_{d}—C(═Y₅)ₑ, are independently selected from among:

—(CR₂₁R₂₂)ₜ₁—[C(═Y₆)]ₑ₁—(Y₄)_{c}—(CR₂R₃)_{d}—C(═Y₅)ₑ—;

—(CR₂₁R₂₂)ₜ₁Y₇—(CR₂₃R₂₄)ₜ₂—(Y₈)ₑ₂—[C(═Y₆)]ₑ₁—(Y₄)_{c}—(CR₂R₃)_{d}—C(═Y₅)ₑ—,

—(CR₂₁R₂₂CR₂₃R₂₄Y₇)ₜ₃—[C(═Y₆)]ₑ₁—(Y₄)_{c}—(CR₂R₃)_{d}—C(═Y₅)ₑ—,

—(CR₂₁R₂₂CR₂₃R₂₄Y₇)ₜ₃(CR₂₅R₂₆)ₜ₄—(Y₈)ₜ₂—[(C(═Y₆)]ₑ₁—(Y₄)_{c}—(CR₂R₃)_{d}—C(═Y₅)ₑ—,

—(CR₂₁R₂₂CR₂₃R₂₄Y₇)ₜ₃(CR₂₅R₂₆)ₜ₄—(Y₈)ₑ₂—[C(═Y₆)]ₑ₁—(CR₂₇R₂₈)ₜ₁—(Y₄)_{c}—(CR₂R₃)_{d}—C(═Y)ₑ—,

—[(CR₂₁R₂₂CR₂₃R₂₄)ₜ₅Y₇]ₜ₆(CR₂₅R₂₆)ₜ₄—(Y₈)ₑ₂—[C(═Y₆)]ₑ₁—(Y₄)_{c}—(CR₂R₃)_{d}—C(αY₅)ₑ—,

—(CR₂₁R₂₂)ₜ₁—[(CR₂₃R₂₄)ₜ₂Y₇]ₜ₇(CR₂₅R₂₆)ₜ₄—(Y₄)_{c}—(CR₂R₃)_{d}—C(═Y₅)ₑ—, and

—(CR₂₁R₂₂)ₜ₁—[(CR₂₃R₂₄)ₜ₂Y₇]ₜ₇(CR₂₅R₂₆)ₜ₄—(Y₈)ₑ₂—[C(═Y₆)]ₑ₁—(Y₄)_{c}—(CR₂R₃)_{d}—C(═Y₅)ₑ—,

wherein:

Y₆ is O, NR₂₉, or S, preferably O;

Y₇₋₈ are independently O, NR₂₉, or S, preferably O or NR₂₉;

R₂₁₋₂₉ are independently selected from the group consisting of hydrogen, C₁₋₆alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆heteroalkyls, C₁₋₆ alkoxy, phenoxy and C₁₋₆heteroalkoxy, preferably H, methyl, ethyl, and propyl, and more preferably H;

each of (t1), (t2), (t3), (t4), (t5), (t6) and (t7) is independently zero or a positive integer (e.g., 1, 2, 3, 4);

each (c), (e), (e1) and (e2) are independently zero or 1; and

all the other variables are as defined above.

The bifunctional L₁ linkers contemplated within the scope of the present invention include those in which combinations of substituents and variables are permissible so that such combinations result in stable compounds (cationic lipids of Formula (I)).

For purposes of the present inventions, R₂₁, R₂₂, R₂₃, R₂₄, R₂₅, R₂₆, R₂₇, and R₂₈, in each occurrence, are independently the same or different when each of (t1), (t2), (t3), (t4), (t5), (t6) and (t7) is independently equal to or greater than 2.

In one preferred embodiment, R₂₁₋₂₉ are hydrogen or methyl.

In another preferred embodiment, Y₇₋₈ are O or NH and R₂₁₋₂₉ are hydrogen or methyl.

In a further embodiment and/or alternative embodiment, illustrative examples of the L₁ group when combined with a moiety of (Y₄)_{c}—(CR₂R₃)_{d}—C(═Y₅)ₑ are selected from among:

—(CH₂)₄—C(═O)—,

—(CH₂)₅—C(═O)—,

—(CH₂)₆—C(═O)—,

—CH₂CH₂O—CH₂O—C(═O)—,

—(CH₂CH₂O)₂—CH₂O—C(═O)—,

—(CH₂CH₂O)₃—CH₂O—C(═O)—,

—(CH₂CH₂O)₂—C(═O)—,

—CH₂CH₂O—CH₂CH₂NH—C(═O)—,

—(CH₂CH₂O)₂—CH₂CH₂NH—C(═O)—,

—(CH₂CH₂O)₂—CH₂CH₂NH—C(═O)—CH₂NHC(═O)—,

—(CH₂CH₂O)₂—CH₂CH₂O—C(═O)—,

—CH₂—O—CH₂CH₂O—CH₂CH₂NH—C(═O)—,

—CH₂—O—(CH₂CH₂O)₂—CH₂CH₂NH—C(═O)—,

—CH₂—O—CH₂CH₂O—CH₂C(═O)—,

—CH₂—O—(CH₂CH₂O)₂—CH₂C(═O)—,

—(CH₂)₄—C(═O)NH—,

—(CH₂)₅—C(═O)NH—,

—(CH₂)₆—C(═O)NH—,

—CH₂CH₂O—CH₂O—C(═O)—NH—,

—(CH₂CH₂O)₂—CH₂O—C(═O)—NH—,

—(CH₂CH₂O)₃—CH₂O—C(═O)—NH—,

—(CH₂CH₂O)₂—C(═O)—NH—,

—(CH₂CH₂O)₂—CH₂C(═O)—NH—,

—CH₂CH₂O—CH₂CH₂NH—C(═O)—NH—,

—(CH₂CH₂O)₂—CH₂CH₂NH—C(═O)—NH—,

—CH₂—O—CH₂CH₂O—CH₂CH₂NH—C(═O)—NH—,

—CH₂—O—(CH₂CH₂O)₂—CH₂CH₂NH—C(═O)—NH—,

—CH₂—O—CH₂CH₂O—CH₂C(═O)—NH—,

—CH₂—O—(CH₂CH₂O)₂—CH₂C(═O)—NH—,

—(CH₂CH₂O)₂—,

—(CH₂CH₂O)₃—,

—CH₂CH₂O—CH₂O—,

—(CH₂CH₂O)₂—CH₂CH₂NH—,

—(CH₂CH₂O)₃—CH₂CH₂NH—,

—CH₂CH₂O—CH₂CH₂NH—,

—(CH₂CH₂O)₂—CH₂CH₂NH—,

—CH₂—O—CH₂CH₂O—CH₂CH₂NH—,

—CH₂—O—(CH₂CH₂O)₂—CH₂CH₂NH—,

—CH₂—O—CH₂CH₂O— and

—CH₂—O—(CH₂CH₂O)₂—.

### 3. Bifunctional Spacers L₁₁₋₁₃ and L′₁₁₋₁₃

In another embodiment, the bifunctional spacers L₁₁₋₁₃ and L′₁₁₋₁₃ are terminal bifunctional linkers which can be connected to cationic moieties, such as guanidinium, DBU, DBN, etc. The bifunctional linkers L₁₁₋₁₃ and L′₁₁₋₁₃ are independently selected from among:

—(CR′₂₁R′₂₂)_{q1}(Y′₈)_{v′}[C(═Y′₉)]ᵥ(CR′₂₃R′₂₄)_{q2}—,

—(CR′₂₁R′₂₂)_{q1}(Y′₈)_{v′}[C(═Y′₉)]ᵥY′₁₀(CR′₂₃R′₂₄)_{q2}—,

—(CR′₂₁R′₂₂)_{q1}(Y′₈)_{v′}[C(═Y′₉)]ᵥ(CR′₂₃R′₂₄)_{q2}—Y′₁₁—(CR′₂₃R′₂₄)_{q3}—,

—(CR′₂₁R′₂₂)_{q1}(Y′₈)_{v′}[C(═Y′₉)]ᵥY′₁₀(CR′₂₃R′₂₄)_{q2}—Y′₁₁—(CR′₂₃R′₂₄)_{q3}—,

—(CR′₂₁R′₂₂)_{q1}(Y′₈)_{v′}[C(═Y′₉)]ᵥ(CR′₂₃R′₂₄R′₂₅R′₂₆Y′₁₂)_{q4}(CR′₂₇CR′₂₈)_{q5}—,

—(CR′₂₁R′₂₂)_{q1}(Y′₈)_{v′}[C(═Y′₉)]ᵥY′₁₀(CR′₂₃R′₂₄CR′₂₅R′₂₆Y′₁₂)_{q4}(CR′₂₇CR′₂₈)_{q5}—, and

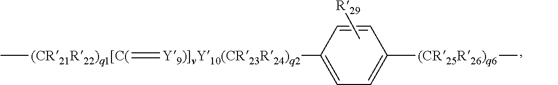

wherein:

Y′₈ and Y′₁₀₋₁₂ are independently O, NR′₃₀, or S, preferably O or NR′₃₀;

Y′₉ are independently O, NR′₃₁, or S, preferably O;

R′₂₁₋₃₁, in each occurrence, are independently selected from among hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy, preferably hydrogen, methyl, ethyl and propyl;

(q1), (q2), (q3), (q4), (q5), and (q6) are independently zero or a positive integer of from about 1 to about 10, preferably 1, 2, 3, 4, 5, 6; and

(v) and (v′) are independently zero or 1.

The bifunctional spacers contemplated within the scope of the present invention include those in which combinations of substituents and variables are permissible so that such combinations result in stable compounds.

For purposes of the present inventions, R′₂₁ and R′₂₂, in each occurrence, are independently the same or different when (q1) is equal to or greater than 2.

For purposes of the present inventions, R′₂₃ and R′₂₄, in each occurrence, are independently the same or different when (q2) and/or (q3) is equal to or greater than 2.

For purposes of the present inventions, R′₂₃, R′₂₄, R′₂₅ and R′₂₆, in each occurrence, are independently the same or different when (q4) is equal to or greater than 2.

For purposes of the present inventions, R′₂₅ and R′₂₅, in each occurrence, are independently the same or different when (q6) is equal to or greater than 2.

For purposes of the present inventions, R′₂₇ and R₂₈, in each occurrence, are independently the same or different when (q5) is equal to or greater than 2.

In a preferred embodiment, R′₂₁₋₃₁ are hydrogen or methyl.

In another preferred embodiment, L₁₁₋₁₃ and L′₁₁₋₁₃ is independently selected from among:

—CH₂—,

—(CH₂)₂—,

—(CH₂)₄—,

—(CH₂)₃—,

—O(CH₂)₂—

—C(═O)O(CH₂)₃—,

—C(═O)NH(CH₂)₃—,

—C(═O)(CH₂)₂—,

—C(═O)(CH₂)₃—,

—CH₂—C(═O)—O(CH₂)₃—,

—CH₂—C(═O)—NH(CH₂)₃—,

—CH₂—OC(═O)—O(CH₂)₃—,

—CH₂—OC(═O)—NH(CH₂)₃—,

—(CH₂)₂—C(═O)—O(CH₂)₃—,

—(CH₂)₂—C(═O)—NH(CH₂)₃—,

—CH₂C(═O)O(CH₂)₂—O—(CH₂)₂—,

—CH₂C(═O)NH(CH₂)₂—O—(CH₂)₂—,

—(CH₂)₂C(═O)O(CH₂)₂—O—(CH₂)₂—,

—(CH₂)₂C(═O)NH(CH₂)₂—O—(CH₂)₂—,

—CH₂C(═O)O(CH₂CH₂O)₂CH₂CH₂—, and

—(CH₂)₂C(═O)O(CH₂CH₂O)₂CH₂CH₂—.

In certain embodiments, some examples of the X(Q₁)(Q₂)(Q₃) moiety include:

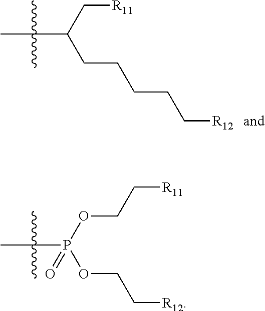

In a preferred embodiment, both R₁₁ and R₁₂ include:

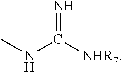

In another preferred embodiment, both R′₁₁ and R′₁₂ include:

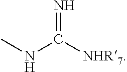

### B. Preparation of Cationic Lipids of Formula (I)

The methods of preparing cationic lipids of Formula (I) described herein include reacting an amine-functionalized cholesterol (functionalized cholesterol) with 1H-pyrazole-1-carboxamidine to provide a guanidinium moiety. The amine linked to cholesterol can be a primary and/or secondary amine and the amines in 1H-pyrazole-1-carboxamidine can be unsubstituted or substituted.

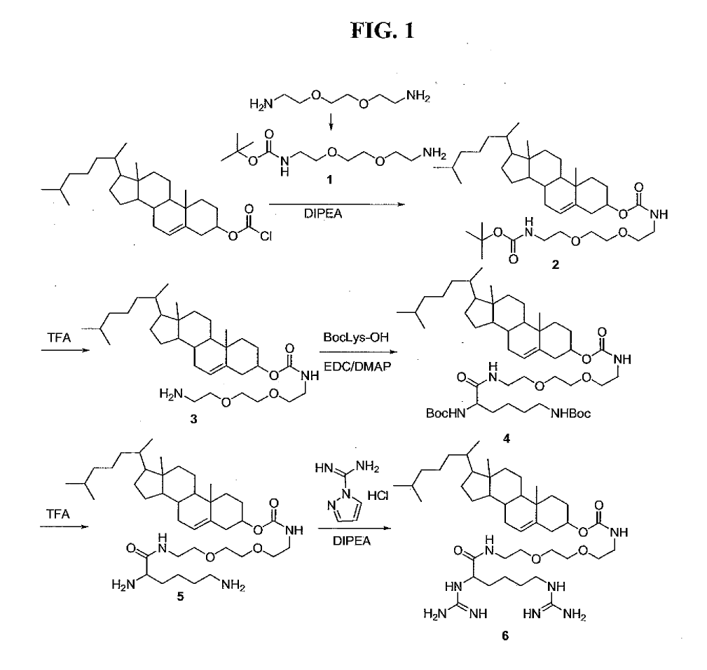

One illustrative example of the preparation of a cholesteryl cationic lipid is shown in FIG. 1. An activated cholesterol carbonate such as cholesteryl chloroformate, cholesteryl NHS carbonate, or cholesteryl PNP carbonate, reacts with a nucleophile amine followed by deprotection of the Boc group to prepare compound 3 (cholesterol having a bifunctional linker with a terminal amine). The terminal amine was further reacted with lysine to prepare cholesterol with a branched moiety (compound 4). By deprotection of the Boc moiety of compound 4 in an acidic condition, compound 5 was prepared. The amines of compound 5 reacted with 1H-pyrazole-1-carboxamidine to provide compound 6 containing bis-guanidinium moieties.

Attachment of an amine-containing compound to cholesterol can be carried out by using standard organic synthetic techniques in the presence of a base, using coupling agents known to those of ordinary skill in the art such as 1,3-diisopropylcarbodiimide (DIPC), dialkyl carbodiimides, 2-halo-1-alkylpyridinium halides, 1-(3-dimethylaminopropyl)-3-ethyl carbodiimide (EDC), propane phosphonic acid cyclic anhydride (PPACA) and phenyl dichlorophosphates.

In a further embodiment, when cholesterol or an amine-containing compound is activated with a leaving group such as NHS, PNP, or chloroformate, the reaction can be carried out in the presence of a base without a coupling agent.

Generally, the cationic lipids of Formula (I) described herein are prepared by reacting an activated cholesterol with an amine-containing nucleophile such as compound 1 in the presence of a base such as DMAP or DIEA. Preferably, the reaction is carried out in an inert solvent such as methylene chloride, chloroform, toluene, DMF or mixtures thereof. The reaction is also preferably conducted in the presence of a base, such as DMAP, DIEA, pyridine, triethylamine, etc., at a temperature from about −4° C. to about 70° C. (e.g. abut −4° C. to about 50° C.). In one preferred embodiment, the reaction is performed at a temperature from about 0° C. to about 25° C. or 0° C. to about room temperature.

Removal of a protecting group from an amine-containing compound, such as compound 2 or 4, can be carried out with a strong acid such as trifluoroacetic acid (TFA), HCl, sulfuric acid, etc., or by catalytic hydrogenation, radical reaction, etc. In one embodiment, deprotection of a Boc group is carried out with HCl solution in dioxane. The deprotection reaction can be carried out at a temperature from about −4° C. to about 50° C. Preferably, the reaction is carried out at a temperature from about 0° C. to about 25° C. or to room temperature. In another embodiment, the deprotection of a Boc group is carried out at room temperature.

Conversion of an amine to a guanidine moiety is carried out by reacting an amine linked to cholesterol (e.g., the amines of compound 5) with 1H-pyrazole-1-carboxamidine in an inert solvent such as methylene chloride, chloroform, DMF or mixtures thereof. Other reagents, such as N-BOC-1H-pyrazole-1-carboxamidine or N,N′-Di-(tert-butoxycarbonyl)thiourea and a coupling reagent can also be used to convert the amine to a guanidine moiety.

Coupling agents known to those of ordinary skill in the art, such as 1,3-diisopropylcarbodiimide (DIPC), dialkyl carbodiimides, 2-halo-1-alkylpyridinium halides, 1-(3-dimethylaminopropyl)-3-ethyl carbodiimide (EDC), propane phosphonic acid cyclic anhydride (PPACA) and phenyl dichlorophosphates, can be employed in the preparation of cationic lipids described herein. The reaction is preferably conducted in the presence of a base, such as DMAP, DIEA, pyridine, triethylamine, etc. at a temperature from about −4° C. to about 50° C. In one preferred embodiment, the reaction is performed at a temperature from about 0° C. to about 25° C. or to room temperature.

Some representative embodiments prepared by the methods described herein include, but are not limited to:

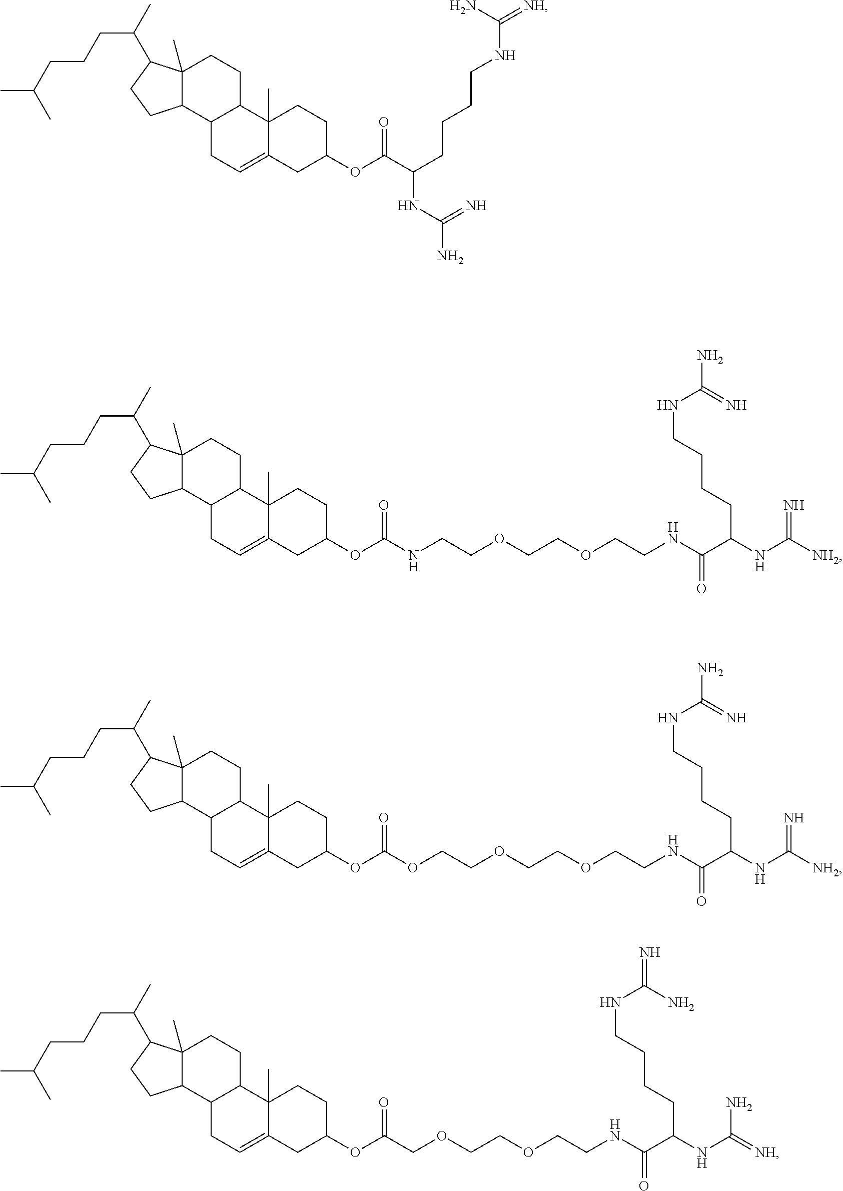

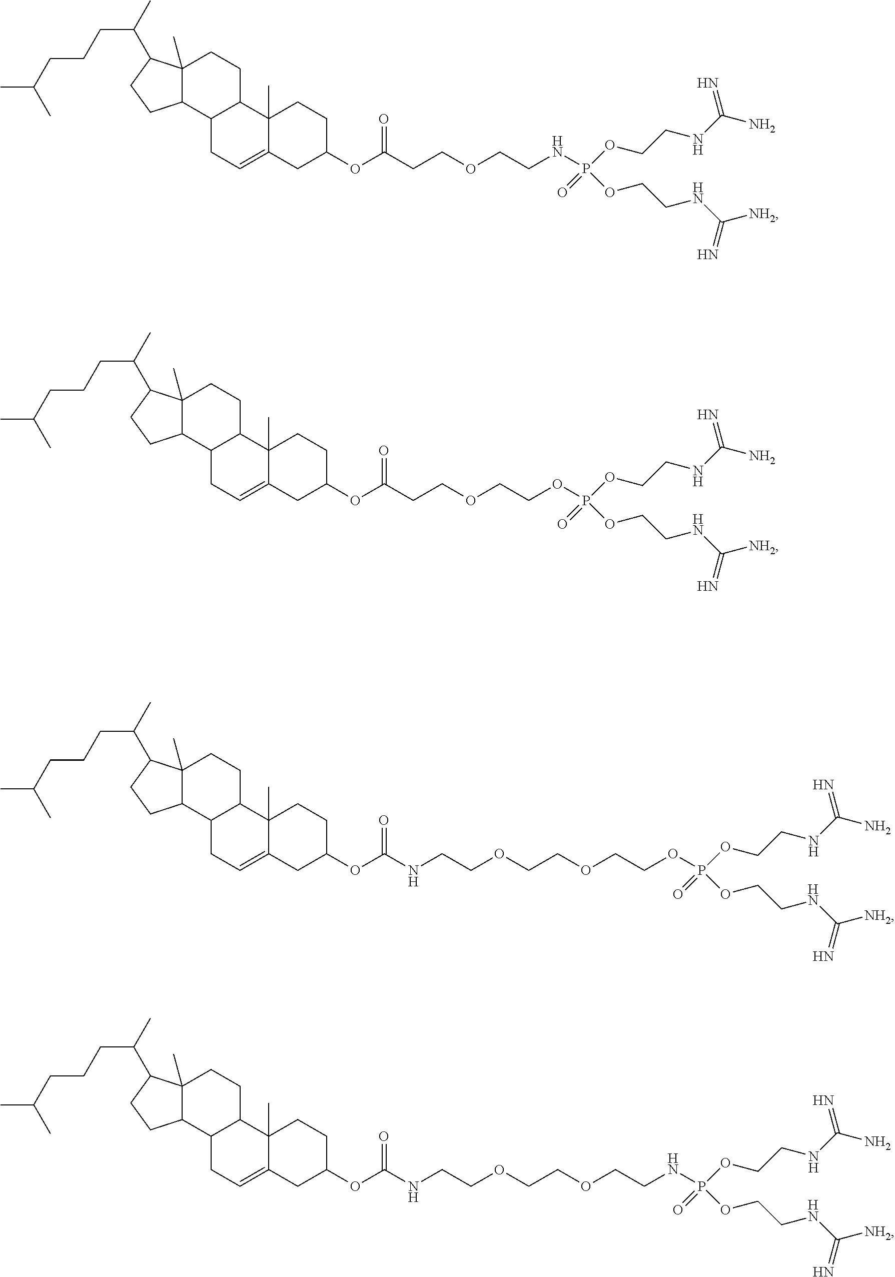

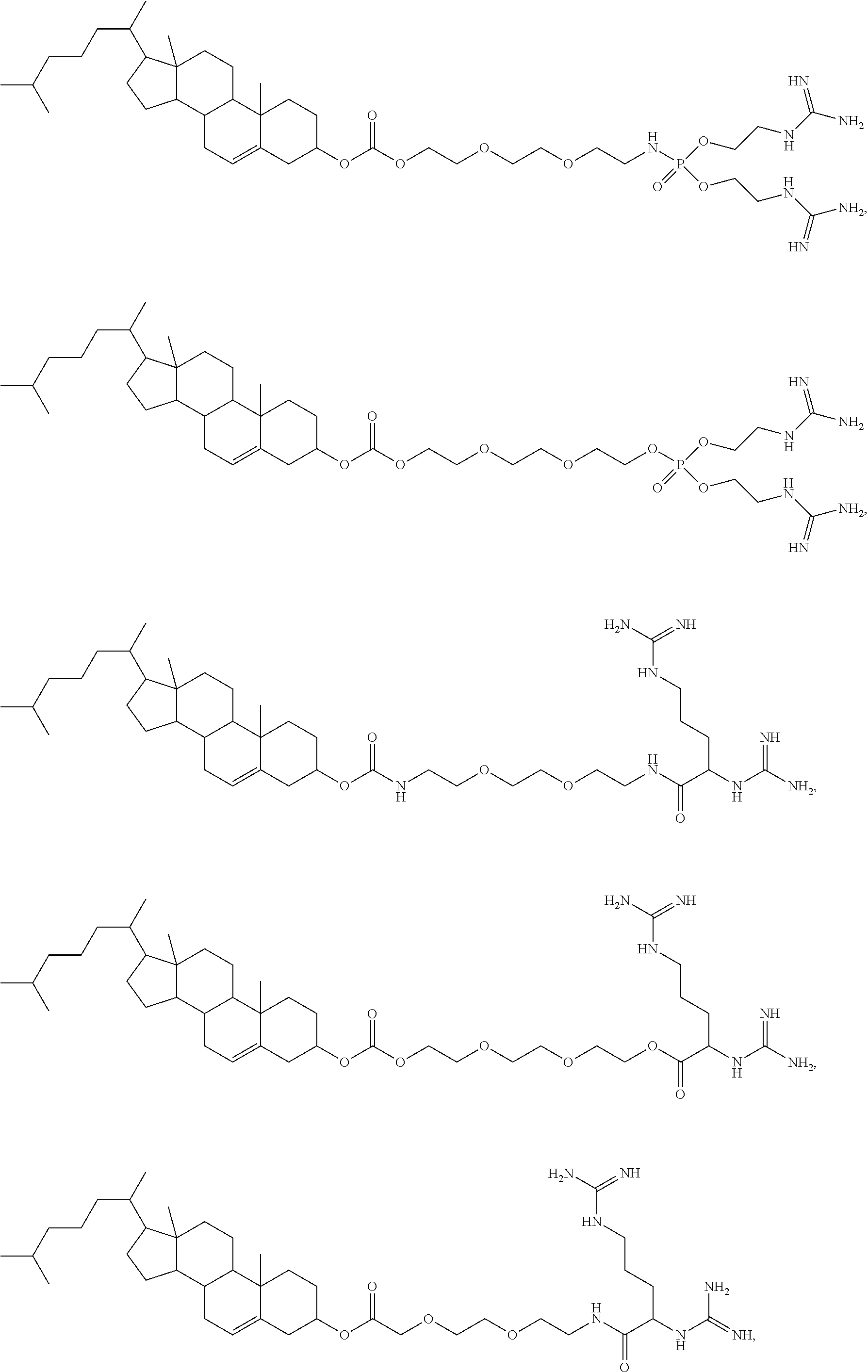

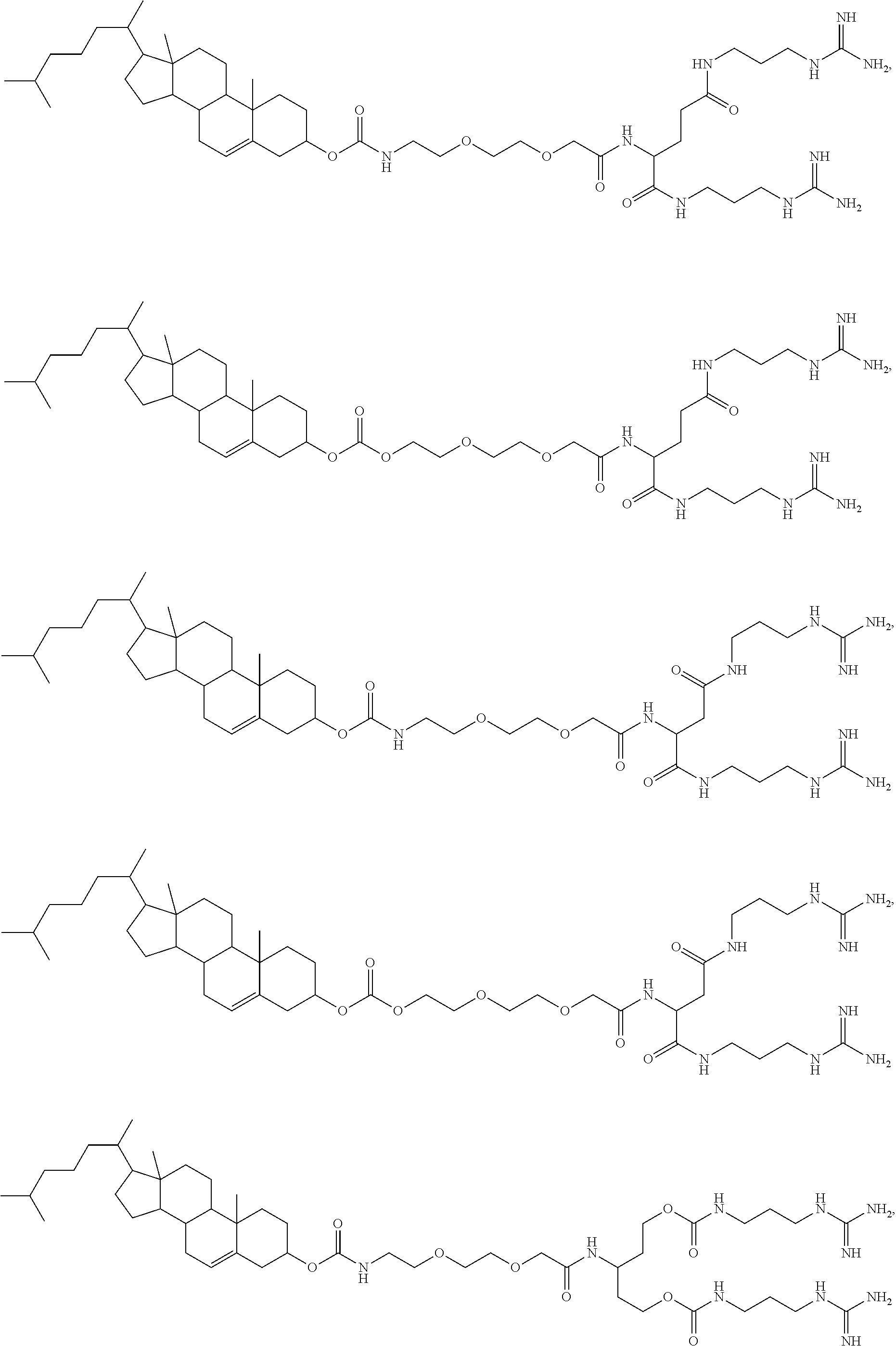

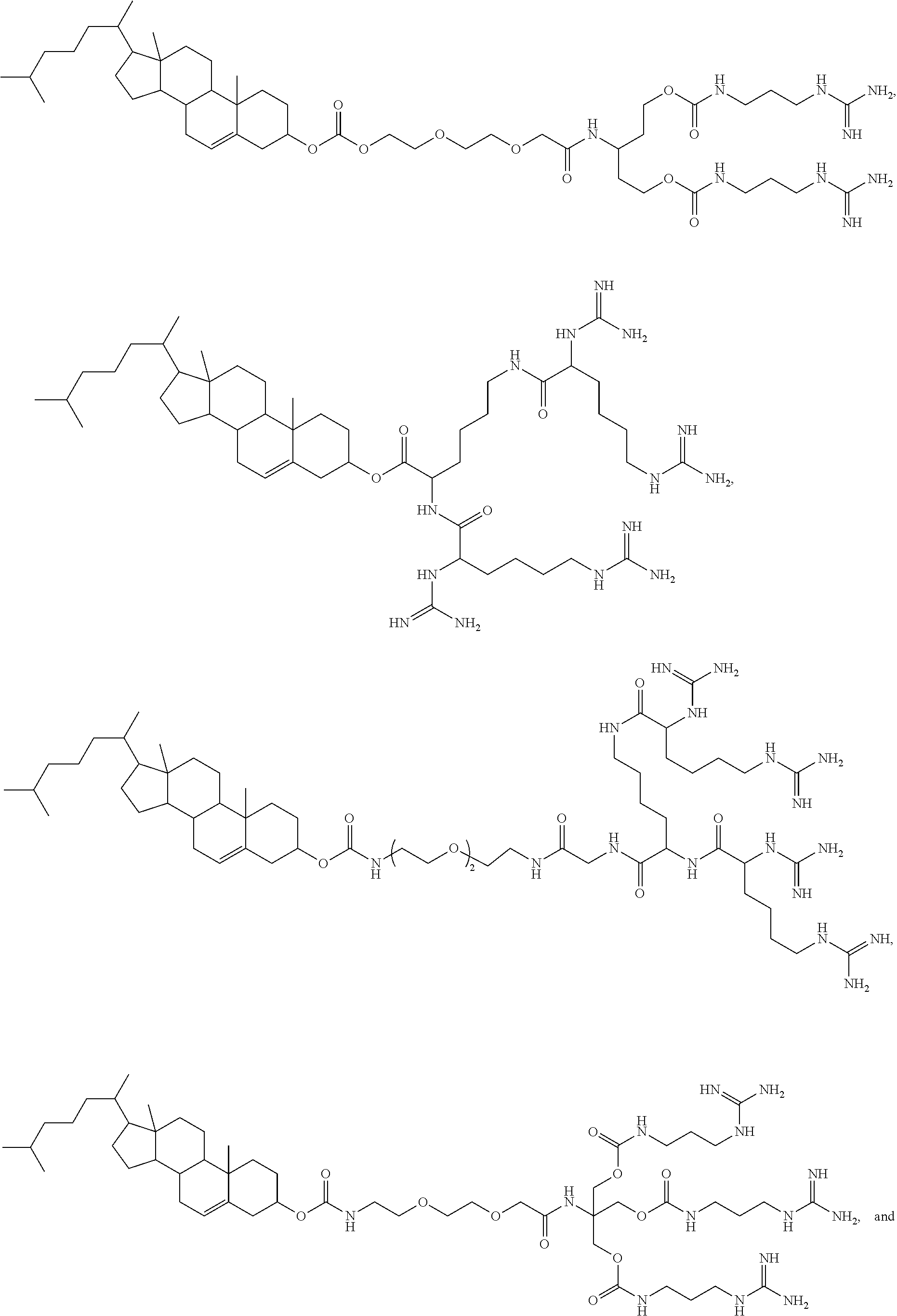

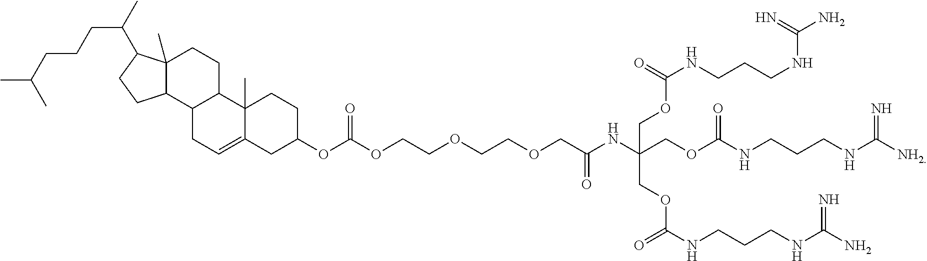

One preferred embodiment includes:

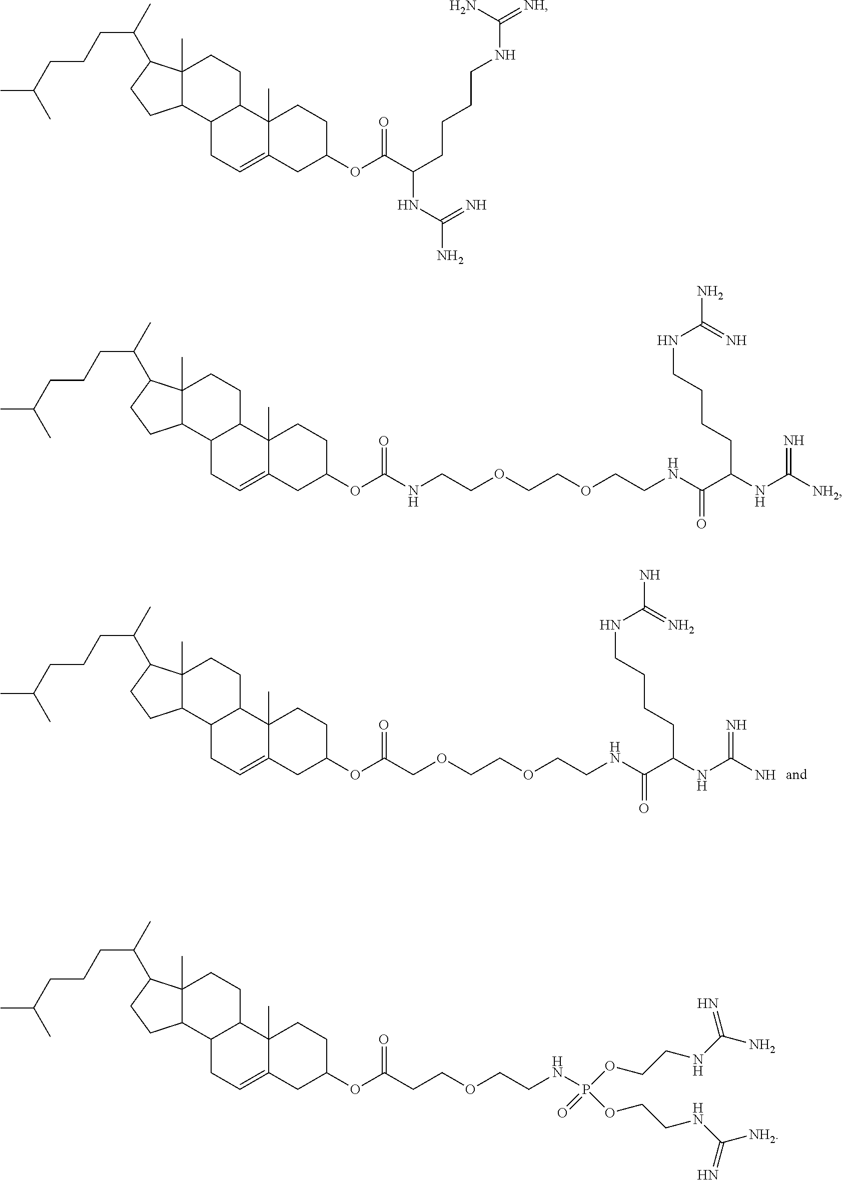

### C. Nanoparticle Compositions/Formulations

### 1. Overview

In one aspect of the invention, the nanoparticle composition contains a cationic lipid of Formula (I).

In a preferred aspect, the nanoparticle composition contains a cationic lipid of Formula (I), a fusogenic lipid and a PEG-lipid.

In a more preferred aspect, the nanoparticle composition includes cholesterol.

In a further aspect of the present invention, the nanoparticle composition described herein may contain additional art-known cationic lipids. The nanoparticle composition containing a mixture of different fusogenic lipids (non-cationic lipids) and/or a mixture of different PEG-lipids are also contemplated.

In another aspect, the nanoparticle composition contains the cationic lipid of Formula (I) described herein in a molar ratio ranging from about 10% to about 99.9% of the total lipid (pharmaceutical carrier) present in the nanoparticle composition.

The cationic lipid component can range from about 2% to about 60%, from about 5% to about 50%, from about 10% to about 45%, from about 15% to about 25%, or from about 30% to about 40% of the total lipid present in the nanoparticle composition.

In one particular embodiment, the cationic lipid is present in amounts of from about 15 to about 25% (i.e., 15, 16, 17, 18, 19, 20, 21, 22, 23, 24 or 25%) of the total lipid present in the nanoparticle composition.

In another preferred aspect of the nanoparticle composition described herein, the compositions contain a total fusogenic/non-cationic lipid, including cholesterol and/or noncholesterol-based fusogenic lipid, in a molar ratio of from about 20% to about 85%, from about 25% to about 85%, from about 60% to about 80% (e.g., 65, 75, 78, or 80%) of the total lipid present in the nanoparticle composition. In one particular embodiment, a total fusogenic/non-cationic lipid is about 80% of the total lipid present in the nanoparticle composition.

In yet another preferred embodiment, a noncholesterol-based fusogenic/non-cationic lipid is present in a molar ratio of from about 25 to about 78% (25, 35, 47, 60, or 78%), or from about 60 to about 78% of the total lipid present in the nanoparticle composition. In one particular embodiment, a noncholesterol-based fusogenic/non-cationic lipid is about 60% of the total lipid present in the nanoparticle composition.

In yet another preferred aspect, the nanoparticle composition includes cholesterol in addition to non-cholesterol fusogenic lipid, in a molar ratio ranging from about 0% to about 60%, from about 10% to about 60%, or from about 20% to about 50% (e.g., 20, 30, 40 or 50%) of the total lipid present in the nanoparticle composition. In one embodiment, cholesterol is about 20% of the total lipid present in the nanoparticle composition.

In yet another aspect of the invention, the PEG-lipid contained in the nanoparticle composition ranges in a molar ratio of from about 0.5% to about 20%, from about 1.5% to about 18% of the total lipid present in the nanoparticle composition. In one embodiment of the nanoparticle composition, the PEG lipid is included in a molar ratio of from about 2% to about 10% (e.g., 2, 3, 4, 5, 6, 7, 8, 9 or 10%) of the total lipid. For example, a total PEG lipid is about 2% of the total lipid present in the nanoparticle composition.

### 2. Cationic Lipids

In one preferred aspect of the invention, the cationic lipids of Formula (I) are included in a nanoparticle composition. In accordance with this aspect of the invention, the nanoparticle composition for the delivery of nucleic acids (i.e., an oligonucleotide) may further include a fusogenic lipid and a PEG lipid.

In a further aspect of the invention, the nanoparticle composition described herein can include additional art-known cationic lipids. Additional suitable lipids contemplated include for example:

N-[1-(2,3-dioleoyloxy)propyl]-N,N,N-trimethylammonium chloride (DOTMA);

1,2-bis(oleoyloxy)-3-3-(trimethylammonium)propane or N-(2,3-dioleoyloxy)propyl)-N,N,N-trimethylammonium chloride (DOTAP);

1,2-bis(dimyrstoyloxy)-3-3-(trimethylammonia)propane (DMTAP);

1,2-dimyristyloxypropyl-3-dimethylhydroxyethylammonium bromide or N-(1,2-dimyristyloxyprop-3-yl)-N,N-dimethyl-N-hydroxyethyl ammonium bromide (DMRIE);

dimethyldioctadecylammonium bromide or N,N-distearyl-N,N-dimethylammonium bromide (DDAB);

3-(N—(N′,N′-dimethylaminoethane)carbamoyl)cholesterol (DC-Cholesterol);

3β-[N′,N′-diguanidinoethyl-aminoethane)carbamoyl cholesterol (BGTC);

2-(2-(3-(bis(3-aminopropyl)amino)propylamino)acetamido)-N,N-ditetradecylacetamide (RPR209120);

1,2-dialkenoyl-sn-glycero-3-ethylphosphocholines (i.e., 1,2-dioleoyl-sn-glycero-3-ethylphosphocholine, 1,2-distearoyl-sn-glycero-3-ethylphosphocholine and 1,2-dipalmitoyl-sn-glycero-3-ethylphosphocholine);

tetramethyltetrapalmitoyl spermine (TMTPS);

tetramethyltetraoleyl spermine (TMTOS);

tetramethlytetralauryl spermine (TMTLS);

tetramethyltetramyristyl spermine (TMTMS);

tetramethyldioleyl spermine (TMDOS);

2,5-bis(3-aminopropylamino)-N-(2-(dioctadecylamino)-2-oxoethyl)pentanamide (DOGS);

2,5-bis(3-aminopropylamino)-N-(2-(di(Z)-octadeca-9-dienylamino)-2-oxoethy-1)pentanamide (DOGS-9-en);

2,5-bis(3-aminopropylamino)-N-(2-(di(9Z,12Z)-octadeca-9,12-dienylamino)-2-oxoethyl)pentanamide (DLinGS);

N4-Spermine cholesteryl carbamate (GL-67);

(9Z,9′Z)-2-(2,5-bis(3-aminopropylamino)pentanamido)propane-1,3-diyl-dioctadec-9-enoate (DOSPER);

2,3-dioleyloxy-N-[2(sperminecarboxamido)ethyl]-N,N-dimethyl-1-propanaminium trifluoroacetate (DOSPA);

1,2-dimyristoyl-3-trimethylammonium-propane; 1,2-distearoyl-3-trimethylammonium-propane;

dioctadecyldimethylammonium (DODMA);

distearyldimethylammonium (DSDMA);

N,N-dioleyl-N,N-dimethylammonium chloride (DODAC); pharmaceutically acceptable salts and mixtures thereof.

Details of cationic lipids are also described in US2007/0293449 and U.S. Pat. Nos. 4,897,355; 5,279,833; 6,733,777; 6,376,248; 5,736,392; 5,686,958; 5,334,761; 5,459,127; 2005/0064595; 5,208,036; 5,264,618; 5,279,833; 5,283,185; 5,753,613; and 5,785,992.

Additionally, commercially available preparations including cationic lipids can be used: for example, LIPOFECTIN® (cationic liposomes containing DOTMA and DOPE, from GIBCO/BRL, Grand Island, N.Y., USA); LIPOFECTAMINE® (cationic liposomes containing DOSPA and DOPE, from GIBCO/BRL, Grand Island, N.Y., USA); and TRANSFECTAM® (cationic liposomes containing DOGS from Promega Corp., Madison, Wis., USA).

### 3. Fusogenic/Non-Cationic Lipids

In another aspect of the invention, the nanoparticle composition contains a fusogenic lipid. The fusogenic lipids include non-cationic lipids such as neutral uncharged, zwitter ionic and anionic lipids. For purposes of the present invention, the terms “fusogenic lipid” and “non-cationic lipids” are interchangeable.

Neutral lipids include a lipid that exists either in an uncharged or neutral zwitter ionic form at a selected pH, preferably at physiological pH. Examples of such lipids include diacylphosphatidylcholine, diacylphosphatidylethanolamine, ceramide, sphingomyelin, cephalin, cholesterol, cerebrosides and diacylglycerols.

Anionic lipids include a lipid that is negatively charged at physiological pH. These lipids include, but are not limited to, phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and neutral lipids modified with other anionic modifying groups.

Many fusogenic lipids include amphipathic lipids generally having a hydrophobic moiety and a polar head group, and can form vesicles in aqueous solution.

Fusogenic lipids contemplated include naturally-occurring and synthetic phospholipids and related lipids.

A non-limiting list of the non-cationic lipids are selected from among phospholipids and nonphosphous lipid-based materials, such as lecithin; lysolecithin; diacylphosphatidylcholine; lysophosphatidylcholine; phosphatidylethanolamine; lysophosphatidylethanolamine; phosphatidylserine; phosphatidylinositol; sphingomyelin; cephalin; ceramide; cardiolipin; phosphatidic acid; phosphatidylglycerol; cerebrosides; dicetylphosphate;

1,2-dilauroyl-sn-glycerol (DLG);

1,2-dimyristoyl-sn-glycerol (DMG);

1,2-dipalmitoyl-sn-glycerol (DPG);

1,2-distearoyl-sn-glycerol (DSG);

1,2-dilauroyl-sn-glycero-3-phosphatidic acid (DLPA);

1,2-dimyristoyl-sn-glycero-3-phosphatidic acid (DMPA);

1,2-dipalmitoyl-sn-glycero-3-phosphatidic acid (DPPA);

1,2-distearoyl-sn-glycero-3-phosphatidic acid (DSPA);

1,2-diarachidoyl-sn-glycero-3-phosphocholine (DAPC);

1,2-dilauroyl-sn-glycero-3-phosphocholine (DLPC);

1,2-dimyristoyl-sn-glycero-3-phosphocholine (DMPC);

1,2-dipalmitoyl-sn-glycero-3-ethylphosphocholine (DPePC);

1,2-dipalmitoyl-sn-glycero-3-phosphocholine or dipalmitoylphosphatidylcholine (DPPC);

1,2-distearoyl-sn-glycero-3-phosphocholine or distearoylphosphatidylcholine (DSPC);

1,2-dilauroyl-sn-glycero-3-phosphoethanolamine (DLPE);

1,2-dimyristoyl-sn-glycero-3-phosphoethanolamine or dimyristoylphosphoethanolamine (DMPE);

1,2-dipalmitoyl-sn-glycero-3-phosphoethanolamine or dipalmitoylphosphatidyl-ethanolamine (DPPE);

1,2-distearoyl-sn-glycero-3-phosphoethanolamine or distearoylphosphatidyl-ethanolamine (DSPE);

1,2-dioleoyl-sn-glycero-3-phosphoethanolamine or dioleoylphosphatidylethanolamine (DOPE);

1,2-dilauroyl-sn-glycero-3-phosphoglycerol (DLPG);

1,2-dimyristoyl-sn-glycero-3-phosphoglycerol (DMPG) or 1,2-dimyristoyl-sn-glycero-3-phospho-sn-1-glycerol (DMP-sn-1-G);

1,2-dipalmitoyl-sn-glycero-3-phosphoglycerol or dipahnitoylphosphatidylglycerol (DPPG);

1,2-distearoyl-sn-glycero-3-phosphoglyeerol (DSPG) or 1,2-distearoyl-sn-glycero-3-phospho-sn-1-glycerol (DSP-sn-1-G);

1,2-dipalmitoyl-sn-glycero-3-phospho-L-serine (DPPS);

1-palmitoyl-2-linoleoyl-sn-glycero-3-phosphocholine (PLinoPC);

1-palmitoyl-2-oleoyl-sn-glycero-3-phosphocholine or palmitoyloleoylphosphatidylcholine (POPC);

1-palmitoyl-2-oleoyl-sn-glycero-3-phosphoglycerol (POPG);

1-palmitoyl-2-lyso-sn-glycero-3-phosphocholine (P-lyso-PC);

1-stearoyl-2-lyso-sn-glycero-3-phosphocholine (S-lyso-PC);

diphytanoylphosphatidylethanolamine (DPhPE);

1,2-dioleoyl-sn-glycero-3-phosphocholine or dioleoylphosphatidylcholine (DOPC);

1,2-diphytanoyl-sn-glycero-3-phosphocholine (DPhPC),

dioleoylphosphatidylglycerol (DOPG);

palmitoyloleoylphosphatidylethanolamine (POPE);

dioleoyl-phosphatidylethanolamine 4-(N-maleimidomethyl)-cyclohexane-1-carboxylate (DOPE-mal);

16-O-monomethyl PE;

16-O-dimethyl PE;

18-1-trans PE; 1-stearoyl-2-oleoyl-phosphatidyethanolamine (SOPE);

1,2-dielaidoyl-sn-glycero-3-phophoethanolamine (transDOPE); and pharmaceutically acceptable salts thereof and mixtures thereof. Details of the fusogenic lipids are described in US Patent Publication Nos. 200710293449 and 2006/0051405.

Noncationic lipids include sterols or steroid alcohols such as cholesterol.

Additional non-cationic lipids are, e.g., stearylamine, dodecylamine, hexadecylamine, acetylpalmitate, glycerolricinoleate, hexadecylstereate, isopropylmyristate, amphoteric acrylic polymers, triethanolaminelauryl sulfate, alkylarylsulfate polyethyloxylated fatty acid amides, and dioctadecyldimethyl ammonium bromide.

Anionic lipids contemplated include phosphatidylserine, phosphatidic acid, phosphatidylcholine, platelet-activation factor (PAF), phosphatidylethanolamine, phosphatidyl-DL-glycerol, phosphatidylinositol, phosphatidylinositol, cardiolipin, lysophosphatides, hydrogenated phospholipids, sphingoplipids, gangliosides, phytosphingosine, sphinganines, pharmaceutically acceptable salts and mixtures thereof.

Suitable noncationic lipids useful for the preparation of the nanoparticle composition described herein include diacylphosphatidylcholine (e.g., distearoylphosphatidylcholine, dioleoylphosphatidylcholine, dipalmitoylphosphatidylcholine and dilinoleoylphosphatidyl-choline), diacylphosphatidylethanolamine (e.g., dioleoylphosphatidylethanolamine and palmitoyloleoylphosphatidylethanolamine), ceramide or sphingomyelin. The acyl groups in these lipids are preferably fatty acids having saturated and unsaturated carbon chains such as linoyl, isostearyl, oleyl, elaidyl, petroselinyl, linolenyl, elaeostearyl, arachidyl, myristoyl, palmitoyl, and lauroyl. More preferably, the acyl groups are lauroyl, myristoyl, palmitoyl, stearoyl or oleoyl. Alternatively and/preferably, the fatty acids have saturated and unsaturated C₈-C₃₀ (preferably C₁₀-C₂₄) carbon chains.

A variety of phosphatidylcholines useful in the nanoparticle composition described herein includes:

1,2-didecanoyl-sn-glycero-3-phosphocholine (DDPC, C10:0, C10:0);

1,2-dilauroyl-sn-glycero-3-phosphocholine (DLPC, C12:0, C12:0);

1,2-dimyristoyl-sn-glycero-3-phosphocholine (DMPC, C14:0, C14:0);

1,2-dipalmitoyl-sn-glycero-3-phosphocholine (DPPC, C16:0, C16:0);

1,2-distearoyl-sn-glycero-3-phosphocholine (DSPC, C18:0, C18:0);

1,2-dioleoyl-sn-glycero-3-phosphocholine (DOPC, C18:1, C18:1);

1,2-dierucoyl-sn-glycero-3-phosphocholine (DEPC, C22:1, C22:1);

1,2-dieicosapentaenoyl-sn-glycero-3-phosphocholine (EPA-PC, C20:5, C20:5);

1,2-didocosahexaenoyl-sn-glycero-3-phosphocholine (DHA-PC, C22:6, C22:6);

1-myristoyl-2-palmitoyl-sn-glycero-3-phosphocholine (MPPC, C14:0, C16:0);

1-myristoyl-2-stearoyl -sn-glycero-3-phosphocholine (MSPC, C14:0, C18:0);

1-palmitoyl-2-stearoyl-sn-glycero-3-phosphocholine (PMPC, C16:0, C14:0);

1-palmitoyl-2-stearoyl-sn-glycero-3-phosphocholine (PSPC, C16:0, C18:0);

1-stearoyl-2-myristoyl-sn-glycero-3-phosphocholine (SMPC, C18:0, C14:0);

1-stearoyl-2-palmitoyl-sn-glycero-3-phosphocholine (SPPC, C18:0, C16:0);

1,2-myristoyl-oleoyl-sn-glycero-3-phosphoethanolamine (MOPC, C14:0, C18:0);

1,2-palmitoyl-oleoyl-sn-glycero-3-phosphoethanolamine (POPC, C16:0, C18:1);

1,2-stearoyl-oleoyl-sn-glycero-3-phosphoethanolamine (POPC, C18:0, C18:1), and pharmaceutically acceptable salts thereof and mixtures thereof.

A variety of lysophosphatidylcholine useful in the nanoparticle composition described herein includes:

1-myristoyl-2-lyso-sn-glycero-3-phosphocholine (M-LysoPC, C14:0);

1-malmitoyl-2-lyso-sn-glycero-3-phosphocholine (P-LysoPC, C16:0);

1-stearoyl-2-lyso-sn-glycero-3-phosphocholine (S-LysoPC, C18:0), and pharmaceutically acceptable salts thereof and mixtures thereof.

A variety of phosphatidylglycerols useful in the nanoparticle composition described herein are selected from among:

hydrogenated soybean phosphatidylglycerol (HSPG);

non-hydrogenated egg phosphatidylgycerol (EPG);

1,2-dimyristoyl-sn-glycero-3-phosphoglycerol (DMPG, C14:0, C14:0);

1,2-dipalmitoyl-sn-glycero-3-phosphoglycerol (DPPG, C16:0, C16:0);

1,2-distearoyl-sn-glycero-3-phosphoglycerol (DSPG, C18:0, C18:0);

1,2-dioleoyl-sn-glycero-3-phosphoglycerol (DOPG, C18:1, C18:1);

1,2-dierucoyl-sn-glycero-3-phosphoglycerol (DEPG, C22:1, C22:1);

1-palmitoyl-2-oleoyl-sn-glycero-3-phosphoglycerol (POPG, C16:0, C18:1), and pharmaceutically acceptable salts thereof and mixtures thereof.

A variety of phosphatidic acids useful in the nanoparticle composition described herein includes:

1,2-dimyristoyl-sn-glycero-3-phosphatidic acid (DMPA, C14:0, C14:0);

1,2-dipalmitoyl-sn-glycero-3-phosphatidic acid (DPPA, C16:0, C16:0);

1,2-distearoyl-sn-glycero-3-phosphatidic acid (DSPA, C18:0, C18:0), and pharmaceutically acceptable salts thereof and mixtures thereof.

A variety of phosphatidylethanolamines useful in the nanoparticle composition described herein includes:

hydrogenated soybean phosphatidylethanolamine (HSPE);

non-hydrogenated egg phosphatidylethanolamine (EPE);

1,2-dimyristoyl-sn-glycero-3-phosphoethanolamine (DMPE, C14:0, C14:0);

1,2-dipalmitoyl-sn-glycero-3-phosphoethanolamine (DPPE, C16:0, C16:0);

1,2-distearoyl-sn-glycero-3-phosphoethanolamine (DSPE, C18:0, C18:0);

1,2-dioleoyl-sn-glycero-3-phosphoethanolamine (DOPE, C18:1, C18:1);

1,2-dioleoyl-sn-glycero-3-phosphoethanolamine (DEPE, C22:1, C22:1);

1,2-dierucoyl-sn-glycero-3-phosphoethanolamine (POPE, C16:0, C18:1), and pharmaceutically acceptable salts thereof and mixtures thereof.

A variety of phosphatidylserines useful in the nanoparticle composition described herein includes:

1,2-dimyristoyl-sn-glycero-3-phospho-L-serine (DMPS, C14:0, C14:0);

1,2-dipalmitoyl-sn-glycero-3-phospho-L-serine (DPPS, C16:0, C16:0);

1,2-distearoyl-sn-glycero-3-phospho-L-serine (DSPS, C18:0, C18:0);

1,2-dioleoyl-sn-glycero-3-phospho-L-serine (DOPS, C18:1, C18:1);

1-palmitoyl-2-oleoyl-sn-3-phospho-L-serine (POPS, C16:0, C18:1), and pharmaceutically acceptable salts thereof and mixtures thereof.

In one preferred embodiment, suitable neutral lipids useful for the preparation of the nanoparticle composition described herein include, for example,

dioleoylphosphatidylethanolamine (DOPE),

distearoylphosphatidylethanolamine (DSPE),

palmitoyloleoylphosphatidylethanolamine (POPE),

egg phosphatidylcholine (EPC),

dipalmitoylphosphatidylcholine (DPPC),

distearoylphosphatidylcholine (DSPC),

dioleoylphosphatidylcholine (DOPC),

palmitoyloleoylphosphatidylcholine (POPC),

dipalmitoylphosphatidylglycerol (DPPG),

dioleoylphosphatidylglycerol (DOPG),

dioleoyl-phosphatidylethanolamine 4-(N-maleimidomethyl)-cyclohexane-1-carboxylate (DOPE-mal), cholesterol, pharmaceutically acceptable salts and mixtures thereof.

In certain preferred embodiments, the nanoparticle composition described herein includes DSPC, EPC, DOPE, etc, and mixtures thereof.

In a further aspect of the invention, the nanoparticle composition contains non-cationic lipids such as sterol. The nanoparticle composition preferably contains cholesterol or analogs thereof, and more preferably cholesterol.

### 4. PEG Lipids

In another aspect of the invention, the nanoparticle composition described herein contains a PEG lipid. The PEG lipids extend circulation of the nanoparticle described herein and prevent the premature excretion of the nanoparticles from the body. The PEG lipids reduce the immunogenicity and enhance the stability of the nanoparticles.

The PEG lipids useful in the nanoparticle composition include PEGylated forms of fusogenic/noncationic lipids. The PEG lipids include, for example, PEG conjugated to diacylglycerol (PEG-DAG), PEG conjugated to diacylglycamides, PEG conjugated to dialkyloxypropyls (PEG-DAA), PEG conjugated to phospholipids such as PEG coupled to phosphatidylethanolamine (PEG-PE), PEG conjugated to ceramides (PEG-Cer), PEG conjugated to cholesterol derivatives (PEG-Chol) or mixtures thereof. See U.S. Pat. Nos. 5,885,613 and 5,820,873, and US Patent Publication No. 2006/051405, the contents of each of which are incorporated herein by reference.

PEG is generally represented by the structure:
—O—(CH₂CH₂O)ₙ—

where (n) is a positive integer from about 5 to about 2300, preferably from about 5 to about 460 so that the polymeric portion of PEG lipid has an average number molecular weight of from about 200 to about 100,000 daltons, preferably from about 200 to about 20,000 daltons. (n) represents the degree of polymerization for the polymer, and is dependent on the molecular weight of the polymer.

In one preferred aspect, the PEG is a polyethylene glycol with a number average molecular weight ranging from about 200 to about 20,000 daltons, more preferably from about 500 to about 10,000 daltons, yet more preferably from about 1,000 to about 5,000 daltons (i.e., about 1,500 to about 3,000 daltons). In one particular embodiment, the PEG has a molecular weight of about 2,000 daltons. In another particular embodiment, the PEG has a molecular weight of about 750 daltons.

Alternatively, the polyethylene glycol (PEG) residue portion can be represented by the structure:

—Y₇₁—(CH₂CH₂O)ₙ—CH₂CH₂Y₇₁—,

—Y₇₁—(CH₂CH₂O)ₙ—CH₂C(═Y₇₂)—Y₇₁—,

—Y₇₁—C(═Y₇₂)—(CH₂)ₐ₂—Y₇₃—(CH₂CH₂O)ₙ—CH₂CH₂—Y₇₃—(CH₂)ₐ₂—C(═Y₇₂)—Y₇₁— and

—Y₇₁—(CR₇₁R₇₂)ₐ₂—Y₇₃—(CH₂)_{b2}—O—(CH₂CH₂O)ₙ—(CH₂)_{b2}—Y₇₃—(CR₇₁R₇₂)ₐ₂—Y₇₁—,

wherein:

Y₇₁ and Y₇₃ are independently O, S, SO, SO₂, NR₇₃ or a bond;

Y₇₂ is O, S, or NR₇₄;

R₇₁₋₇₄ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆heteroalkyl, C₁₋₆alkoxy, aryloxy, C₁₋₆heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy, preferably hydrogen, methyl, ethyl or propyl;

(a2) and (b2) are independently zero or a positive integer, preferably zero or an integer from about 1 to about 6 (i.e., 1, 2, 3, 4, 5, 6), and more preferably 1 or 2; and

(n) is an integer from about 5 to about 2300, preferably from about 5 to about 460.

The terminal end of PEG can end with H, NH₂, OH, CO₂H, C₁₋₆ alkyl (e.g., methyl, ethyl, propyl), C₁₋₆ alkoxy, acyl or aryl. In a preferred embodiment, the terminal hydroxyl group of PEG is substituted with a methoxy or methyl group. In one preferred embodiment, the PEG employed in the PEG lipid is methoxy PEG.

The PEG may be directly conjugated to lipids or via a linker moiety. The polymers for conjugation to a lipid structure are converted into a suitably activated polymer, using the activation techniques described in U.S. Pat. Nos. 5,122,614 and 5,808,096 and other techniques known in the art without undue experimentation.

Examples of activated PEGs useful for the preparation of a PEG lipid include, for example, methoxypolyethylene glycol-succinate, mPEG-NHS, methoxypolyethylene glycol-succinimidyl succinate, methoxypolyethyleneglycol-acetic acid (mPEG-CH₂COOH), methoxypolyethylene glycol-amine (mPEG-NH₂), and methoxypolyethylene glycol-tresylate (mPEG-TRES).

In certain aspects, polymers having terminal carboxylic acid groups can be employed in the PEG lipids described herein. Methods of preparing polymers having terminal carboxylic acids in high purity are described in U.S. patent application Ser. No. 11/328,662, the contents of which are incorporated herein by reference.

In alternative aspects, polymers having terminal amine groups can be employed to make the PEG-lipids described herein. The methods of preparing polymers containing terminal amines in high purity are described in U.S. patent application Ser. Nos. 11/508,507 and 11/537,172, the contents of each of which are incorporated by reference.

PEG and lipids can be bound via a linkage, i.e. a non-ester containing linker moiety or an ester containing linker moiety. Suitable non-ester containing linkers include, but are not limited to, an amido linker moiety, an amino linker moiety, a carbonyl linker moiety, a carbamate linker moiety, a carbonate (OC(═O)O) linker moiety, a urea linker moiety, an ether linker moiety, a succinyl linker moiety, and combinations thereof. Suitable ester linker moieties include, e.g., succinoyl, phosphate esters (—O—P(O)(OH)—O—), sulfonate esters, and combinations thereof.

In one embodiment, the nanoparticle composition described herein includes a polyethyleneglycol-diacylglycerol (PEG-DAG) or polyethylene-diacylglycamide. Suitable polyethyleneglycol-diacylglycerol or polyethyleneglycol-diacylglycamide conjugates include a dialkylglycerol or dialkylglycamide group having alkyl chain length independently containing from about C₄ to about C₃₀ (preferably from about C₈ to about C₂₄) saturated or unsaturated carbon atoms. The dialkylglycerol or dialkylglycamide group can further include one or more substituted alkyl groups.

The term “diacylglycerol” (DAG) used herein refers to a compound having two fatty acyl chains, R₁₁ and R₁₂. The R₁₁ and R₁₂ have the same or different carbon chain in length of about 4 to about 30 carbons (preferably about 8 to about 24) and are bonded to glycerol by ester linkages. The acyl groups can be saturated or unsaturated with various degrees of unsaturation. DAG has the general formula:

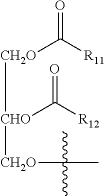

In a preferred embodiment, the PEG-diacylglycerol conjugate is a PEG-dilaurylglycerol (C12), a PEG-dimyristylglycerol (C14, DMG), a PEG-dipalmitoylglycerol (C16, DPG) or a PEG-distearylglycerol (C18, DSG). Those of skill in the art will readily appreciate that other diacylglycerols are also contemplated in the PEG-diacylglycol conjugate. Suitable PEG-diacylglycerol conjugates for use in the present invention, and methods of making and using them, are described in U.S. Patent Publication No. 2003/0077829, and PCT Patent Application No. CA 02/00669, the contents of each of which are incorporated herein by reference.

Examples of the PEG-diacylglycerol conjugate can be selected from among PEG-dilaurylglycerol (C12), PEG-dimyristylglycerol (C14), PEG-dipalmitoylglycerol (C16), PEG-disterylglycerol (C18). Examples of the PEG-diacylglycamide conjugate includes PEG-dilaurylglycamide (C12), PEG-dimyristylglycamide (C14), PEG-dipalmitoyl-glycamide (C16), and PEG-disterylglycamide (C18).

In another embodiment, the nanoparticle composition described herein includes a polyethyleneglycol-dialkyloxypropyl conjugates (PEG-DAA).

The term “dialkyloxypropyl” refers to a compound having two alkyl chains, R₁₁ and R₁₂. The R₁₁ and R₁₂ alkyl groups include the same or different carbon chain length between about 4 to about 30 carbons (preferably about 8 to about 24). The alkyl groups can be saturated or have varying degrees of unsaturation. Dialkyloxypropyls have the general formula:

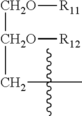

wherein R₁₁ and R₁₂ alkyl groups are the same or different alkyl groups having from about 4 to about 30 carbons (preferably about 8 to about 24). The alkyl groups can be saturated or unsaturated. Suitable alkyl groups include, but are not limited to, lauryl (C12), myristyl (C14), palmityl (C16), stearyl (C18), oleoyl (C18) and icosyl (C20).

In one embodiment, R₁₁ and R₁₂ are both the same, i.e., R₁₁ and R₁₂ are both myristyl (C14), both stearyl (C18) or both oleoyl (C18), etc. In another embodiment, R₁₁ and R₁₂ are different, i.e., R₁₁ is myristyl (C14) and R₁₂ is stearyl (C18). In a preferred embodiment, the PEG-dialkylpropyl conjugates include the same R₁₁ and R₁₂.

In yet another embodiment, the nanoparticle composition described herein includes PEG conjugated to phosphatidylethanolamines (PEG-PE). The phosphatidylethanolaimes useful for the PEG lipid conjugation can contain saturated or unsaturated fatty acids with carbon chain lengths in the range of about 4 to about 30 carbons (preferably about 8 to about 24). Suitable phosphatidylethanolamines include, but are not limited to: dimyristoylphosphatidylethanolamine (DMPE), dipalmitoylphosphatidylethanolamine (DPPE), dioleoylphosphatidylethanolamine (DOPE) and distearoylphosphatidylethanolarnine (DSPE).

In yet another embodiment, the nanoparticle composition described herein includes PEG conjugated to ceramides (PEG-Cer). Ceramides have only one acyl group. Ceramides can have saturated or unsaturated fatty acids with carbon chain lengths in the range of about 4 to about 30 carbons (preferably about 8 to about 24).

In alternative embodiments, the nanoparticle composition described herein includes PEG conjugated to cholesterol derivatives. The term “cholesterol derivative” means any cholesterol analog containing a cholesterol structure with modification, i.e., substitutions and/or deletions thereof. The term cholesterol derivative herein also includes steroid hormones and bile acids.

Illustrative examples of PEG lipids include N-(carbonyl-methoxypolyethyleneglycol)-1,2-dimyristoyl-sn-glycero-3-phosphoethanolamine (^{2 kDa}mPEG-DMPE or ^{5 kDa}mPEG-DMPE); N-(carbonyl-methoxypolyethyleneglycol)-1,2-dipalmitoyl-sn-glycero-3-phosphoethanolamine (^{2 kDa}mPEG-DPPE or ^{5 kDa}mPEG-DPPE), N-(carbonyl-methoxypolyethyleneglycol)-1,2-distearoyl-sn-glycero-3-phosphoethanolamine (^{750 Da}mPEG-DSPE, ^{2 kDa}mPEG-DSPE, ^{5 kDa}mPEG-DSPE); and pharmaceutically acceptable salts therof (i.e., sodium salt) and mixtures thereof.

In certain preferred embodiments, the nanoparticle composition described herein includes a PEG lipid having PEG-DAG or PEG-ceramide, wherein PEG has molecular weight from about 200 to about 20,000, preferably from about 500 to about 10,000, and more preferably from about 1,000 to about 5,000.

A few illustrative embodiments of PEG-DAG and PEG-ceramide are provided in Table 1.

TABLE 1
PEG-Lipid

| | |
|---|---|
| PEG-DAG | mPEG-diimyristoylglycerol |
| | mPEG-dipalmitoylglycerol |
| | mPEG-distearoylglycerol |
| PEG-Ceramide | mPEG-CerC8 |
| | mPEG-CerC14 |
| | mPEG-CerC16 |
| | mPEG-CerC20 |

Preferably, the nanoparticle composition described herein includes the PEG lipid selected from among PEG-DSPE, PEG-dipalmitoylglycamide (C16), PEG-Ceramide (C16), etc. and mixtures thereof. The structures of mPEG-DSPE, mPEG-dipalmitoylglycamide (C16), and mPEG-Ceramide (C16) are as follows:

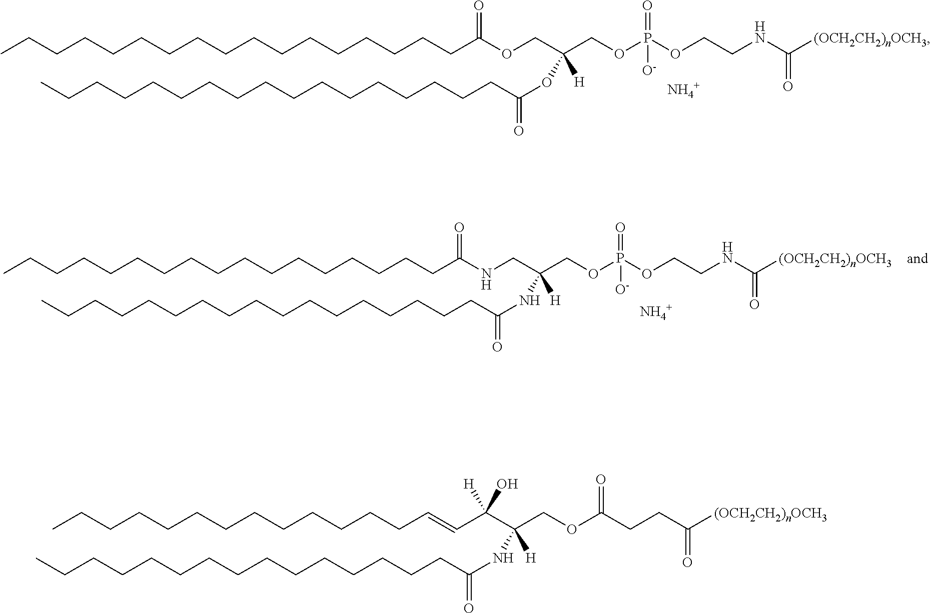

wherein, (n) is an integer from about 5 to about 2300, preferably from about 5 to about 460.

In one preferred embodiment, (n) is about 45.

In a further embodiment and as an alternative to PAO-based polymers such as PEG, one or more effectively non-antigenic materials such as dextran, polyvinyl alcohols, carbohydrate-based polymers, hydroxypropylmethacrylamide (HPMA), polyalkylene oxides, and/or copolymers thereof can be used. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose. See also commonly-assigned U.S. Pat. No. 6,153,655, the contents of which are incorporated herein by reference. It will be understood by those of ordinary skill that the same type of activation can be employed as described herein as for PAOs such as PEG. Those of ordinary skill in the art will further realize that the foregoing list is merely illustrative and that all polymeric materials having the qualities described herein are contemplated. For purposes of the present invention, “substantially or effectively non-antigenic” means all materials understood in the art as being nontoxic and not eliciting an appreciable immunogenic response in mammals.

### 5. Nucleic Acids/Oligonucleotides

The nanoparticle compositions described herein can be used for delivering various nucleic acids into cells or tissues. The nucleic acids include plasmids and oligonucleotides. Preferably, the nanoparticle compositions described herein are used for delivery of oligonucleotides.

In order to more fully appreciate the scope of the present invention, the following terms are defined. The artisan will appreciate that the terms, “nucleic acid” or “nucleotide” apply to deoxyribonucleic acid (“DNA”), ribonucleic acid, (“RNA”) whether single-stranded or double-stranded, unless otherwise specified, and to any chemical modifications or analogs thereof, such as, locked nucleic acids (LNA). The artisan will readily understand that by the term “nucleic acid,” included are polynucleic acids, derivates, modifications and analogs thereof. An “oligonucleotide” is generally a relatively short polynucleotide, e.g., ranging in size from about 2 to about 200 nucleotides, preferably from about 8 to about 50 nucleotides, more preferably from about 8 to about 30 nucleotides, and yet more preferably from about 8 to about 20 or from about 15 to about 28 in length. The oligonucleotides according to the invention are generally synthetic nucleic acids, and are single stranded, unless otherwise specified. The terms, “polynucleotide” and “polynucleic acid” may also be used synonymously herein.

The oligonucleotides (analogs) are not limited to a single species of oligonucleotide but, instead, are designed to work with a wide variety of such moieties, it being understood that linkers can attach to one or more of the 3′- or 5′-terminals, usually PO₄ or SO₄ groups of a nucleotide. The nucleic acid molecules contemplated can include a phosphorothioate internucleotide linkage modification, sugar modification, nucleic acid base modification and/or phosphate backbone modification. The oligonucleotides can contain natural phosphorodiester backbone or phosphorothioate backbone or any other modified backbone analogues such as LNA (Locked Nucleic Acid), PNA (nucleic acid with peptide backbone), CpG oligomers, and the like, such as those disclosed at Tides 2002, Oligonucleotide and Peptide Technology Conferences, May 6-8, 2002, Las Vegas, Nev. and Oligonucleotide & Peptide Technologies, 18 & 19 Nov. 2003, Hamburg, Germany, the contents of which are incorporated herein by reference.

Modifications to the oligonucleotides contemplated by the invention include, for example, the addition or substitution of functional moieties that incorporate additional charge, polarizability, hydrogen bonding, electrostatic interaction, and functionality to an oligonucleotide. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodouracil, backbone modifications, methylations, base-pairing combinations such as the isobases isocytidine and isoguanidine, and analogous combinations. Oligonucleotides contemplated within the scope of the present invention can also include 3′ and/or 5′ cap structure

For purposes of the present invention, “cap structure” shall be understood to mean chemical modifications, which have been incorporated at either terminus of the oligonucleotide. The cap can be present at the 5′-terminus (5′-cap) or at the 3′-terminus (3′-cap) or can be present on both termini. A non-limiting example of the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide; 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety. Details are described in WO 97/26270, the contents of which are incorporated by reference herein. The 3′-cap can include for example 4′,5′-methylene nucleotide; 1-(beta-D-erythrofuranosyl) nucleotide; 4′-thio nucleotide, carbocyclic nucleotide; 5′-aminoalkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide;5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties. See also Beaucage and Iyer, 1993, Tetrahedron 49, 1925; the contents of which are incorporated by reference herein.

A non-limiting list of nucleoside analogs have the structure:

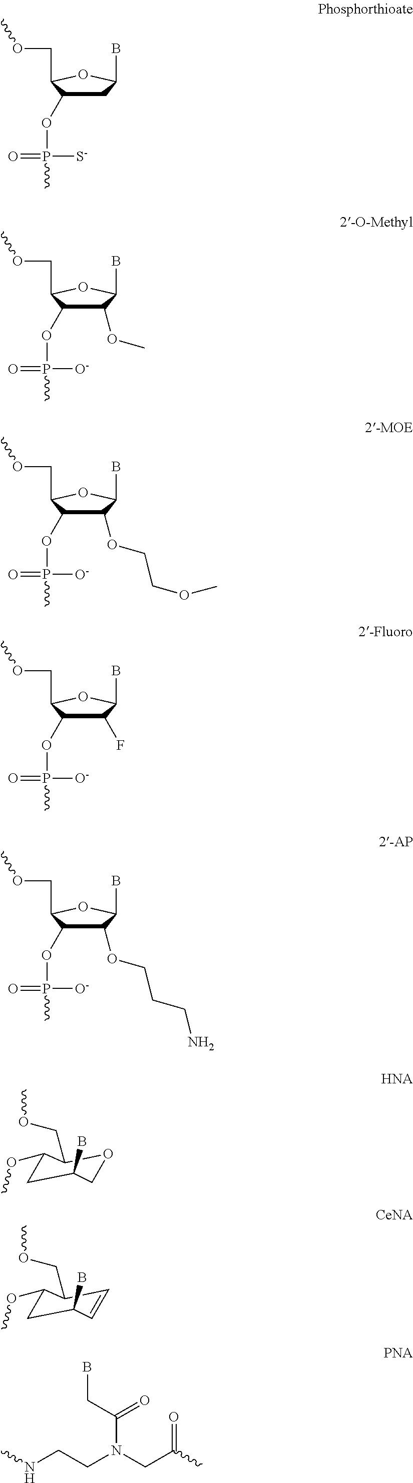

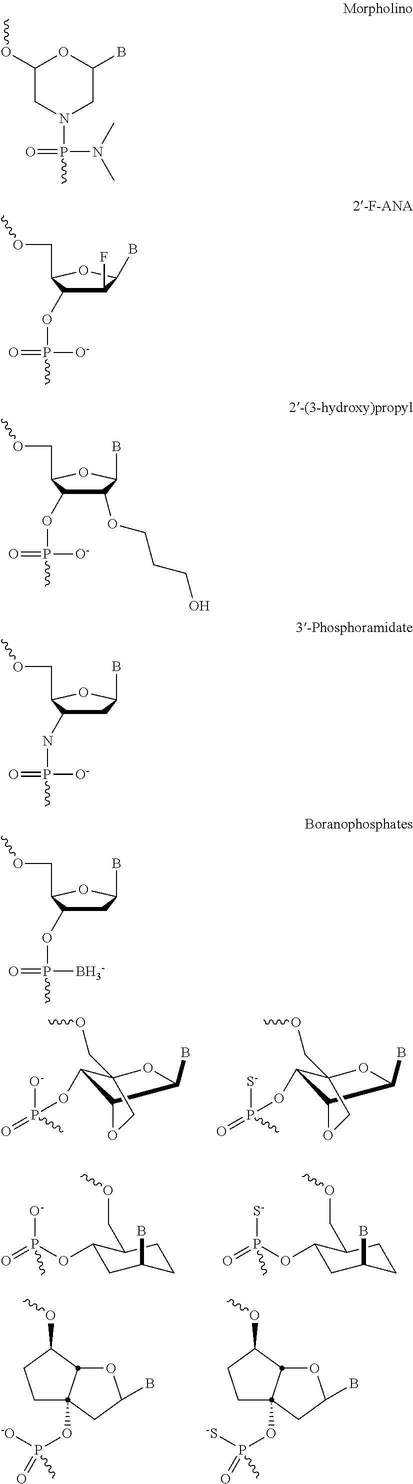

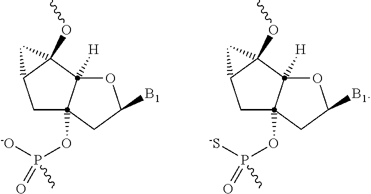

See more examples of nucleoside analogues described in Freier & Altmann; Nucl. Acid Res., 1997, 25, 4429-4443 and Uhlmann; Curr. Opinion in Drug Development, 2000, 3(2), 293-213, the contents of each of which are incorporated herein by reference.

The term “antisense,” as used herein, refers to nucleotide sequences which are complementary to a specific DNA or RNA sequence that encodes a gene product or that encodes a control sequence. The term “antisense strand” is used in reference to a nucleic acid strand that is complementary to the “sense” strand. In the normal operation of cellular metabolism, the sense strand of a DNA molecule is the strand that encodes polypeptides and/or other gene products. The sense strand serves as a template for synthesis of a messenger RNA (“mRNA”) transcript (an antisense strand) which, in turn, directs synthesis of any encoded gene product. Antisense nucleic acid molecules may be produced by any art-known methods, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription of the mRNA or its translation. The designations “negative” or (−) are also art-known to refer to the antisense strand, and “positive” or (+) are also art-known to refer to the sense strand.

For purposes of the present invention, “complementary” shall be understood to mean that a nucleic acid sequence forms hydrogen bond(s) with another nucleic acid sequence. A percent complementarity indicates the percentage of contiguous residues in a nucleic acid molecule which can form hydrogen bonds, i.e., Watson-Crick base pairing, with a second nucleic acid sequence, i.e., 5, 6, 7, 8, 9, 10 out of 10 being 50%, 60%, 70%, 80%, 90%, and 100% complementary. “Perfectly complementary” means that all the contiguous residues of a nucleic acid sequence form hydrogen bonds with the same number of contiguous residues in a second nucleic acid sequence.

The nucleic acids (such as one or more same or differen oligonucleotides or oligonucloetide derivatives) useful in the nanoparticle described herein can include from about 5 to about 1000 nucleic acids, and preferably relatively short polynucleotides, e.g., ranging in size preferably from about 8 to about 50 nucleotides in length (e.g., about 8, 9, 10, 11, 12, 13, 14, 15, 16, 17, 18, 19, 20, 21, 22, 23, 24, 25, 26, 27, 28, 29 or 30).

In one aspect of useful nucleic acids encapsulated within the nanoparticle described herein, oligonucleotides and oligodeoxynucleotides with natural phosphorodiester backbone or phosphorothioate backbone or any other modified backbone analogues include:

LNA (Locked Nucleic Acid);

PNA (nucleic acid with peptide backbone);

short interfering RNA (siRNA);

microRNA (miRNA);

nucleic acid with peptide backbone (PNA);

phosphorodiamidate morpholino oligonucleotides (PMO);

tricyclo-DNA;

decoy ODN (double stranded oligonucleotide);

catalytic RNA sequence (RNAi);

ribozymes;

aptamers;

spiegelmers (L-conformational oligonucleotides);

CpG oligomers, and the like, such as those disclosed at:

Tides 2002, Oligonucleotide and Peptide Technology Conferences, May 6-8, 2002, Las Vegas, Nev. and Oligonucleotide & Peptide Technologies, 18 & 19 Nov. 2003, Hamburg, Germany, the contents of which are incorporated herein by reference.

In another aspect of the nucleic acids encapsulated within the nanoparticle, oligonucleotides can optionally include any suitable art-known nucleotide analogs and derivatives, including those listed by Table 2, below:

TABLE 2
Representative Nucleotide Analogs And Derivatives

| | |
|---|---|
| 4-acetylcytidine | 5-methoxyaminomethyl- |
| | 2-thiouridine |
| 5-(carboxyhydroxymethyl)uridine | beta, D-mannosylqueuosine |
| 2′-O-methylcytidine | 5-methoxycarbonylmethyl- |
| | 2-thiouridine |
| 5-methoxycarbonylmethyluridine | 5-carboxymethylaminomethyl- |
| | 2-thiouridine |
| 5-methoxyuridine | 5-carboxymethylaminomethyluridine |
| Dihydrouridine | 2-methylthio-N6- |
| | isopentenyladenosine |
| 2′-O-methylpseudouridine | N-[(9-beta-D-ribofuranosyl- |
| | 2-methylthiopurine-6- |
| | yl)carbamoyl]threonine |
| D-galactosylqueuosine | N-[(9-beta-D- |
| | ribofuranosylpurine-6-yl)N- |
| | methylcarbamoyl]threonine |
| 2′-O-methylguanosine | uridine-5-oxyacetic |
| | acid-methylester |
| 2′-halo-adenosine | 2′-halo-cytidine |
| 2′-halo-guanosine | 2′-halo-thymine |
| 2′-halo-uridine | 2′-halo-methylcytidine |
| 2′-amino-adenosine | 2′-amino-cytidine |
| 2′-amino-guanosine | 2′-amino-thymine |
| 2′-amino-uridine | 2′-amino-methylcytidine |
| Inosine | uridine-5-oxyacetic acid |
| N6-isopentenyladenosine | Wybutoxosine |
| 1-methyladenosine | Pseudouridine |
| 1-methylpseudouridine | Queuosine |
| 1-methylguanosine | 2-thiocytidine |
| 1-methylinosine | 5-methyl-2-thiouridine |
| 2,2-dimethylguanosine | 2-thiouridine |
| 2-methyladenosine | 4-thiouridine |
| 2-methylguanosine | 5-methyluridine |
| 3-methylcytidine | N-[(9-beta-D- |
| | ribofuranosylpurine-6-yl)- |
| | carbamoyl]threonine |
| 5-methylcytidine | 2′-O-methyl-5-methyluridine |
| N6-methyladenosine | 2′-O-methyluridine |
| 7-methylguanosine | Wybutosine |
| 5-methylaminomethyluridine | 3-(3-amino-3- |
| | carboxy-propyl)uridine |
| Locked-adenosine | Locked-cytidine |
| Locked-guanosine | Locked-thymine |
| Locked-uridine | Locked-methylcytidine |

In one preferred aspect, the target oligonucleotides encapsulated in the nanoparticles include, for example, but are not limited to, oncogenes, pro-angiogenesis pathway genes, pro-cell proliferation pathway genes, viral infectious agent genes, and pro-inflammatory pathway genes.

In one preferred embodiment, the oligonucleotide encapsulated within the nanoparticle described herein is involved in targeting tumor cells or downregulating a gene or protein expression associated with tumor cells and/or the resistance of tumor cells to anticancer therapeutics. For example, antisense oligonucleotides for downregulating any art-known cellular proteins associated with cancer, e.g., BCL-2 can be used for the present invention. See U.S. patent application Ser. No. 10/822,205 filed Apr. 9, 2004, the contents of which are incorporated by reference herein. A non-limiting list of preferred therapeutic oligonucleotides includes antisense HIF1-α oligonucleotides, antisense survivin oligonucleotides, antisense ErbB3 oligonucleotides, antisense β-catenin oligonucleotides and antisense Bcl-2 oligonucleotides.

More preferably, the oligonucleotides according to the invention described herein include phosphorothioate backbone and LNA.

In one preferred embodiment, the oligonucleotide can be, for example, antisense survivin LNA, antisense ErbB3 LNA, or antisense HIF1-α LNA.

In another preferred embodiment, the oligonucleotide can be, for example, an oligonucleotide that has the same or substantially similar nucleotide sequence as does Genasense® (a/k/a oblimersen sodium, produced by Genta Inc., Berkeley Heights, N.J.). Genasense® is an 18-mer phosphorothioate antisense oligonucleotide (SEQ ID NO: 4), that is complementary to the first six codons of the initiating sequence of the human bcl-2 mRNA (human bcl-2 mRNA is art-known, and is described, e.g., as SEQ ID NO: 19 in U.S. Pat. No. 6,414,134, incorporated by reference herein).

Preferred embodiments contemplated include:

(i) antisense Survivin LNA, Oligo-1 (SEQ ID NO: 1)
^{m}Cₛ-Tₛ-^{m}Cₛ-Aₛ-aₛ-tₛ-cₛ-cₛ-aₛ-tₛ-gₛ-gₛ-^{m}Cₛ-Aₛ-Gₛ-c;
where the upper case letter represents LNA, the “s” represents a phosphorothioate backbone;

(ii) antisense Bcl2 siRNA:

(SEQ ID NO: 2)
SENSE 5′-gcaugcggccucuguuugadTdT-3′
(SEQ ID NO: 3)
ANTISENSE 3′-dTdTcguacgccggagacaaacu-5′

where dT represents DNA;

(iii) Genasense (phosphorothioate antisense oligonucleotide): (SEQ ID NO: 4)
tₛ-cₛ-tₛ-cₛ-cₛ-cₛ-aₛ-gₛ-cₛ-gₛ-tₛ-gₛ-cₛ-gₛ-cₛ-cₛ-cₛ-aₛ-t
where the lower case letter represents DNA and and “s” represents phosphorothioate backbone;

(iv) antisense HIF1α LNA (SEQ ID NO: 5)
5′-ₛTₛGₛGₛcₛaₛaₛgₛcₛaₛtₛcₛcₛTₛGₛTₛa-3′
where the upper case letter represents LNA and the “s” represents phosphorothioate backbone.

(v) antisense ErbB3 LNA, Oligo-2 (SEQ ID NO: 6)
5′-TₐAₛGₛcₛcₛtₛgₛtₛcₛaₛcₛtₛtₛCₛTₛCₛ-3′
where the upper case letter represents LNA and the “s” represents phosphorothioate backbone.

LNA includes 2′-O,4′-C methylene bicyclonucleotide as shown below:

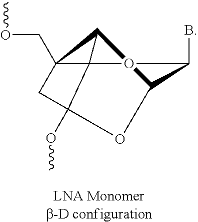

A scrambled antisense ErbB3 LNA, Oligo-3 (SEQ ID NO: 7) has the sequence of:
5′-TAGcttgtcccatt^{m}CT^{m}C-3′
where the upper case letter represents LNA, ^{m}C represents methylated cytosine, and the internucleoside linkage is phosphorothioate.

See detailed description of Survivin LNA disclosed in U.S. Patent Application Publication Nos. 2006/0154888, entitled “LNA Oligonucleotides and the Treatment of Cancer” and 2005/0014712, entitled “Oligomeric Compounds for the Modulation Survivin Expression”, the contents of each of which is incorporated herein by reference. See also U.S. Patent Application Publication Nos. 2004/0096848, entitled “Oligomeric Compounds for the Modulation HIF-1 Alpha Expression” and 2006/0252721, entitled “Potent LNA Oligonucleotides for Inhibition of HIF-1A Expression”, the contents of which are also incorporated herein by reference. See also, the contents of which are incorporated herein by reference in its entirety.

Examples of suitable target genes are described in PCT Publication No. WO 03/74654, PCT/US03/05028, and U.S. patent application Ser. No. 2007/0042983, the contents of which are incorporated by reference herein.

### 6. Targeting Groups

Optionally/preferably, the nanoparticle compositions described herein further include a targeting ligand for a specific cell or tissue type. The targeting group can be attached to any component of a nanoparticle composition (preferably, fusogenic lipids and PEG-lipids) using a linker molecule, such as an amide, amido, carbonyl, ester, peptide, disulphide, silane, nucleoside, abasic nucleoside, polyether, polyamine, polyimide, peptide, carbohydrate, lipid, polyhydrocarbon, phosphate ester, phosphoramidate, thiophosphate, alkylphosphate, maleimidyl linker or photolabile linker. Any known techniques in the art can be used for conjugating a targeting group to any component of the nanoparticle composition without undue experimentation.

For example, targeting agents can be attached to the polymeric portion of PEG lipids to guide the nanoparticles to the target area in vivo. The targeted delivery of the nanoparticle described herein enhances the cellular uptake of the nanoparticles encapsulating therapeutic nucleic acids, thereby improving the therapeutic efficacies. In certain aspects, some cell penetrating peptides can be replaced with a variety of targeting peptides for targeted delivery to the tumor site.

In one preferred aspect of the invention, the targeting moiety, such as a single chain antibody (SCA) or single-chain antigen-binding antibody, monoclonal antibody, cell adhesion peptides such as RGD peptides and Selectin, cell penetrating peptides (CPPs) such as TAT, Penetratin and (Arg)₉, receptor ligands, targeting carbohydrate molecules or lectins allows nanoparticles to be specifically directed to targeted regions. See J Pharm Sci. 2006 September; 95(9):1856-72 Cell adhesion molecules for targeted drug delivery, the contents of which are incorporated herein by reference.

Preferred targeting moieties include single-chain antibodies (SCAs) or single-chain variable fragments of antibodies (sFv). The SCA contains domains of antibodies which can bind or recognize specific molecules of targeting tumor cells. In addition to maintaining an antigen binding site, a SCA conjugated to a PEG-lipid can reduce antigenicity and increase the half life of the SCA in the bloodstream.

The terms “single chain antibody” (SCA), “single-chain antigen-binding molecule or antibody” or “single-chain Fv” (sFv) are used interchangeably. The single chain antibody has binding affinity for the antigen. Single chain antibody (SCA) or single-chain Fvs can and have been constructed in several ways. A description of the theory and production of single-chain antigen-binding proteins is found in commonly assigned U.S. patent application Ser. No. 10/915,069 and U.S. Pat. No. 6,824,782, the contents of each of which are incorporated by reference herein.

Typically, SCA or Fv domains can be selected among monoclonal antibodies known by their abbreviations in the literature as 26-10, MOPC 315, 741F8, 520C9, McPC 603, D1.3, murine phOx, human phOx, RFL3.8 sTCR, 1A6, Se155-4,18-2-3,4-4-20,7A4-1, B6.2, CC49,3C2,2c, MA-15C5/K₁₂G_{O}, Ox, etc. (see, Huston, J. S. et al., Proc. Natl. Acad. Sci. USA 85:5879-5883 (1988); Huston, J. S. et al., SIM News 38(4) (Supp):11 (1988); McCartney, J. et al., ICSU Short Reports 10:114 (1990); McCartney, J. E. et al., unpublished results (1990); Nedehnan, M. A. et al., J. Nuclear Med. 32 (Supp.):1005 (1991); Huston, J. S. et al., In: Molecular Design and Modeling: Concepts and Applications, Part. B, edited by J. J. Langone, Methods in Enzymology 203:46-88 (1991); Huston, J. S. et al., In: Advances in the Applications of Monoclonal Antibodies in Clinical Oncology, Epenetos, A. A. (Ed.), London, Chapman & Hall (1993); Bird, R. E. et al., Science 242:423-426 (1988); Bedzyk, W. D. et al., J. Biol. Chem. 265:18615-18620 (1990); Colcher, D. et al., J. Nat. Cancer Inst. 82:1191-1197 (1990); Gibbs, R. A. et al., Proc. Natl. Acad. Sci. USA 88:4001-4004 (1991); Milenic, D. E. et al., Cancer Research 51:6363-6371 (1991); Pantoliano, M. W. et al., Biochemistry 30:10117-10125 (1991); Chaudhary, V. K. et al., Nature 339:394-397 (1989); Chaudhary, V. K. et al., Proc. Natl. Acad. Sci. USA 87:1066-1070 (1990); Batra, J. K. et al., Biochem. Biophys. Res. Comm. 171:1-6 (1990); Batra, J. K. et al., J. Biol. Chem. 265:15198-15202 (1990); Chaudhary, V. K. et al., Proc. Natl. Acad Sci. USA 87:9491-9494 (1990); Batra, J. K. et al., Mol. Cell. Biol. 11:2200-2205 (1991); Brinkmann, U. et al., Proc. Natl. Acad. Sci. USA 88:8616-8620 (1991); Seetharam, S. et al., J. Biol. Chem. 266:17376-17381 (1991); Brinkmann, U. et al., Proc. Natl. Acad. Sci. USA 89:3075-3079 (1992); Glockshuber, R. et al., Biochemistry 29:1362-1367 (1990); Skerra, A. et al., Bio/Technol. 9:273-278 (1991); Pack, P. et al., Biochemistry 31:1579-1534 (1992); Clackson, T. et al., Nature 352:624-628 (1991); Marks, J. D. et al., J. Mol. Biol. 222:581-597 (1991); Iverson, B. L. et al., Science 249:659-662 (1990); Roberts, V. A. et al., Proc. Natl. Acad. Sci. USA 87:6654-6658 (1990); Condra, J. H. et al., J. Biol. Chem. 265:2292-2295 (1990); Laroche, Y. et al., J. Biol. Chem. 266:16343-16349 (1991); Holvoet, P. et al., J. Biol. Chem. 266:19717-19724 (1991); Anand, N. N. et al., J. Biol. Chem. 266:21874-21879 (1991); Fuchs, P. et al., Biol Technol. 9:1369-1372 (1991); Breitling, F. et al., Gene 104:104-153 (1991); Seehaus, T. et al., Gene 114:235-237 (1992); Takkinen, K. et al., Protein Engng. 4:837-841 (1991); Dreher, M. L. et al., J. Immunol. Methods 139:197-205 (1991); Mottez, E. et al., Eur. J. Immunol. 21:467-471 (1991); Traunecker, A. et al., Proc. Natl. Acad. Sci. USA 88:8646-8650 (1991); Traunecker, A. et al., EMBO J. 10:3655-3659 (1991); Hoo, W. F. S. et al., Proc. Natl. Acad. Sci. USA 89:4759-4763 (1993)). Each of the foregoing publications is incorporated herein by reference.

A non-limiting list of targeting groups includes vascular endothelial cell growth factor, FGF2, somatostatin and somatostatin analogs, transferrin, melanotropin, ApoE and ApoE peptides, von Willebrand's Factor and von Willebrand's Factor peptides, adenoviral fiber protein and adenoviral fiber protein peptides, PD1 and PD1 peptides, EGF and EGF peptides, RGD peptides, folate, etc. Other optional targeting agents appreciated by artisans in the art can be also employed in the nanoparticles described herein.

In one preferred embodiment, the targeting agents useful for the nanoparticle described herein include single chain antibody (SCA), RGD peptides, selectin, TAT, penetratin, (Arg)₉, folic acid, anisamide, etc. and some of the preferred structures of these agents are:

C-TAT: (SEQ ID NO: 8) CYGRKKRRQRRR;

C-(Arg)₉: (SEQ ID NO: 9) CRRRRRRRRR;

RGD can be linear or cyclic:

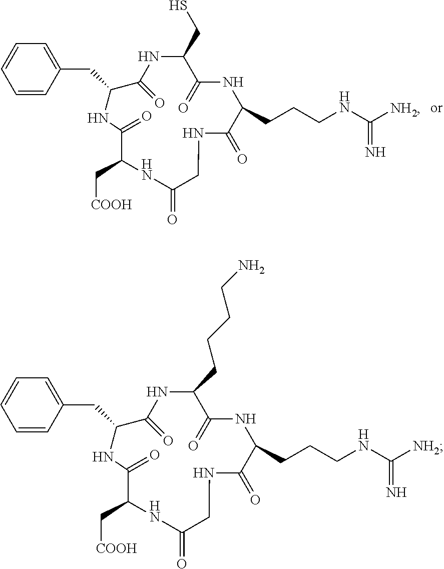

and

Folic acid is a residue of

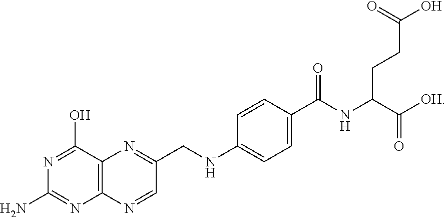

Arg₉ can include a cysteine for conjugating such as CRRRRRRRRR and TAT can add an additional cysteine at the end of the peptide such as CYGRKKRRQRRRC (SEQ ID NO: 10).

For purpose of the current invention, the abbreviations used in the specification and figures represent the following structures.:

(i) C-diTAT (SEQ ID NO: 11)=CYGRKKRRQRRRYGRKKRRQRRR—NH₂;

(ii) Linear RGD (SEQ ID NO: 12)=RGDC;

(iii) Cyclic RGD (SEQ ID NO: 13)=c-RGDFC;

(iv) RGD-TAT (SEQ ID NO: 14) CYGRKKRRQRRRGGGRGDS—NH₂; and

(v) Arg₉ (SEQ ID NO: 15).

Alternatively, the targeting group includes sugars and carbohydrates such as galactose, galactosamine, and N-acetyl galactosamine; hormones such as estrogen, testosterone, progesterone, glucocortisone, adrenaline, insulin, glucagon, cortisol, vitamin D, thyroid hormone, retinoic acid, and growth hormones; growth factors such as VEGF, EGF, NGF, and PDGF; neurotransmitters such as GABA, glutamate, acetylcholine; NOGO; inostitol triphosphate; epinephrine; norepinephrine; nitric oxide, peptides, vitamins such as folate and pyridoxine, drugs, antibodies and any other molecule that can interact with a receptor in vivo or in vitro.

### D. Preparation of Nanoparticles

The nanoparticle described herein can be prepared by any art-known process without undue experimentation.

For example, the nanoparticle can be prepared by providing nucleic acids such as oligonucleotides in an aqueous solution (or an aqueous solution without nucleic acids for comparison study) in a first reservoir, providing an organic lipid solution containing the nanoparticle composition described herein in a second reservoir, and mixing the aqueous solution with the organic lipid solution such that the organic lipid solution mixes with the aqueous solution to produce nanoparticles encapsulating the nucleic acids. Details of the process are described in U.S. Patent Publication No. 2004/0142025, the contents of which are incorporated herein by reference.

Alternatively, the nanoparticles described herein can be prepared by using any methods known in the art including, e.g., a detergent dialysis method or a modified reverse-phase method which utilizes organic solvents to provide a single phase during mixing the components. In a detergent dialysis method, nucleic acids (i.e., siRNA) are contacted with a detergent solution of cationic lipids to form a coated nucleic acid complex.

In one embodiment of the invention, the cationic lipids and nucleic acids such as oligonucleotides are combined to produce a charge ratio of from about 1:20 to about 20:1, preferably in a ratio of from about 1:5 to about 5:1, and more preferably in a ratio of from about 1:2 to about 2:1.

In one embodiment of the invention, the cationic lipids and nucleic acids such as oligonucleotides are combined to produce a charge ratio of from about 1:1 to about 20:1, from about 1:1 to about 12:1, and more preferably in a ratio of from about 2:1 to about 6:1. Alternatively, the nitrogen to phoshpate (N/P) ratio of the nanoparticle composition ranges from about 2:1 to about 5:1, (i.e., 2.5:1).

In another embodiment, the nanoparticle described herein can be prepared by using a dual pump system. Generally, the process includes providing an aqueous solution containing nucleic acids in a first reservoir and a lipid solution containing the nanoparticle composition described in a second reservoir. The two solutions are mixed by using a dual pump system to provide nanoparticles. The resulting mixed solution is subsequently diluted with an aqueous buffer and the nanoparticles formed can be purified and/or isolated by dialysis. The nanoparticles can be further processed to be sterilized by filtering through a 0.22 μm filter.

The nanoparticles containing nucleic acids range from about 5 to about 300 nm in diameter. Preferably, the nanoparticles have a median diameter of less than about 150 nm (e.g., about 50-150 nm), more preferably a diameter of less than about 100 nm, by the measurement using the Dynamic Light Scattering technique (DLS). A majority of the nanoparticles have a median diameter of about 30 to 100 nm (e.g., 59.5, 66, 68, 76, 80, 93, 96 nm), preferably about 60 to about 95 nm. Artisans will appreciate that the measurement using other art-known techniques such as TEM may provide a median diameter number decreased by half, as compared to the DLS technique. The nanoparticles of the present invention are substantially uniform in size as shown by polydispersity.

Optionally, the nanoparticles can be sized by any methods known in the art. The size can be controlled as desired by artisans. The sizing may be conducted in order to achieve a desired size range and relatively narrow distribution of nanoparticle sizes. Several techniques are available for sizing the nanoparticles to a desired size. See, for example, U.S. Pat. No. 4,737,323, the contents of which are incorporated herein by reference.

The present invention provides methods for preparing serum-stable nanoparticles such that nucleic acids (e.g., LNA or siRNA) are encapsulated in a lipid multi-lamellar structure (i.e. a lipid bilayer) and are protected from degradation. The nanoparticles described herein are stable in an aqueous solution. Nucleic acids included in the nanoparticles are protected from nucleases present in the body fluid.

Additionally, the nanoparticles prepared according to the present invention are preferably neutral or positively-charged at physiological pH.

The nanoparticle or nanoparticle complex prepared using the nanoparticle composition described herein includes: (i) a cationic lipid of Formula (I); (ii) a neutral lipid/fusogenic lipid; (iii) a PEG-lipid and (iv) nucleic acids such as an oligonucleotide.

In one embodiment, the nanoparticle composition includes a mixture of

a cationic lipid of Formula (I), a diacylphosphatidylethanolarnine, a PEG conjugated to phosphatidylethanolamine (PEG-PE), and cholesterol;

a cationic lipid of Formula (I), a diacylphosphatidylcboline, a PEG conjugated to phosphatidylethanolamine (PEG-PE), and cholesterol;

a cationic lipid of Formula (I), a diacylphosphatidylethanolamine, a diacylphosphatidyl-choline, a PEG conjugated to phosphatidylethanolamine (PEG-PE), and cholesterol;

a cationic lipid of Formula (I), a diacylphosphatidylethanolamine, a PEG conjugated to ceramide (PEG-Cer), and cholesterol; or

a cationic lipid of Formula (I), a diacylphosphatidylethanolamine, a PEG conjugated to phosphatidylethanolamine (PEG-PE), a PEG conjugated to ceramide (PEG-Cer), and cholesterol.

Additional nanoparticle compositions can be prepared by modifying compositions containing art-known cationic lipid(s). Nanoparticle compositions containing art-known cationic lipid(s) can be modified by replacing art-known cationic lipids with a cationic lipid of Formula (I) and/or adding a cationic lipid of Formula (1). See art-known compositions described in Table IV of US Patent Application Publication No. 2008/0020058, the contents of which are incorporated herein by reference.

A non-limiting list of nanoparticle compositions for the preparation of nanoparticles is set forth in Table 3.

TABLE 3

| Sample | | | |
|---|---|---|---|
| No. | Nanoparticle Composition | Molar Ratio | Oligo |
| 1 | Compd 6: DOPE: DSPC: | 15:15:20:40:10 | Oligo-1 |
| | Chol: DSPE-PEG | | |
| 2 | Compd 6: DOPE: DSPC: | 15:5:20:50:10 | Oligo-1 |
| | Chol: DSPE-PEG | | |
| 3 | Compd 6: DOPE: DSPC: | 25:15:20:30:10 | Oligo-1 |
| | Chol: DSPE-PEG | | |
| 4 | Compd 6: EPC: Chol: DSPE-PEG | 20:47:30:3 | Oligo-1 |
| 5 | Compd 6: DOPE: Chol: DSPE-PEG | 17:60:20:3 | Oligo-1 |
| 6 | Compd 6: DOPE: DSPE-PEG | 20:78:2 | Oligo-1 |
| 7 | Compd 6: DOPE: Chol: | 17:60:20:3 | Oligo-2 |
| | C16mPEG-Ceramide | | |
| 8 | Compd 6: DOPE: Chol: DSPE- | 18:60:20:1:1 | Oligo-2 |
| | PEG: C16mPEG-Ceramide | | |

In one embodiment, the molar ratio of a cationic lipid (compound 6): DOPE: cholesterol: PEG-DSPE: C16mPEG-Ceramide in the nanoparticle is in a molar ratio of about 18%: 60%:20%:1%:1%, respectively, based the total lipid present in the nanoparticle composition (Sample No. 8).

In another embodiment, the nanoparticle contains a cationic lipid (compound 6), DOPE, cholesterol and C16mPEG-Ceramide in a molar ratio of about 17%:60%:20%:3% of the total lipid present in the nanoparticle composition (Sample No. 7)

These nanoparticle compositions preferably contain a cationic lipid having the structure:

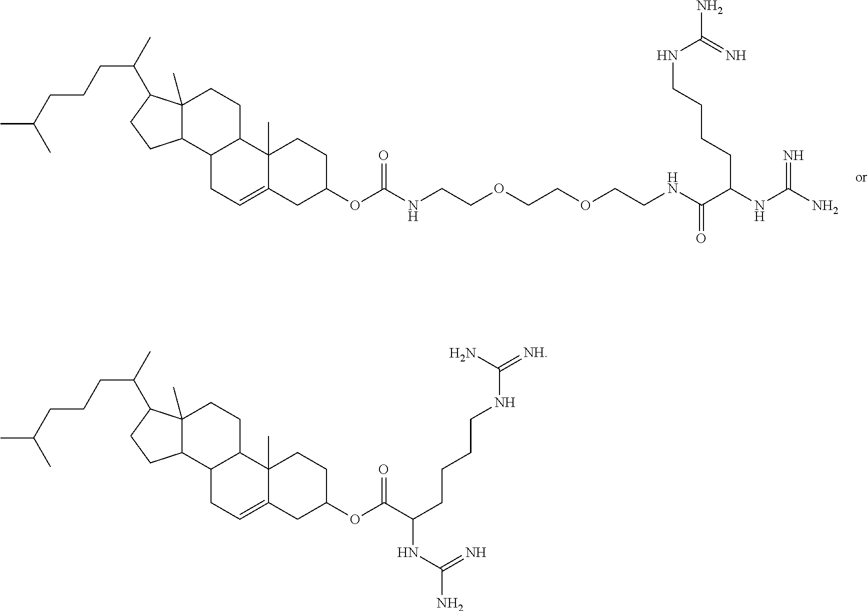

The molar ratio as used herein refers to the amount relative to the total lipid present in the nanoparticle composition.

### E. Methods of Treatment

The nanoparticles described herein can be employed in the treatment for preventing, inhibiting, reducing or treating any trait, disease or condition that is related to or responds to the levels of target gene expression in a cell or tissue, alone or in combination with other therapies. The method includes administering the nanoparticle described herein to a mammal in need thereof.

One aspect of the present invention provides methods of introducing or delivering therapeutic nucleic acids such as oligonucleotides into a mammalian cell in vivo and/or in vitro.

The method according to the present invention includes contacting a cell with the nanoparticle described herein. The delivery can be made in vivo as part of a suitable pharmaceutical composition or directly to the cells in an ex vivo environment.

In another aspect, the present invention is useful for introducing oligonucleotides to a mammal. The nanoparticles described herein can be administered to a mammal, preferably human.

In yet antoher aspect, the present invention preferably provides methods of inhibiting, or downregulating (or modulating) a gene expression in mammalian cells or tissues. The downregulation or inhibition of gene expression can be achieved in vivo, ex vivo and/or in vitro. The methods include contacting human cells or tissues with nanoparticles encapsulating nucleic acids described herein or administering the nanoparticles in a mammal in need thereof. Once the contacting has occurred, successful inhibition or down-regulation of gene expression such as in mRNA or protein levels shall be deemed to occur when at least about 10%, preferably at least about 20% or higher (e.g., at least about 25%, 30%, 40%, 50%, 60%) is realized in vivo, ex vivo or in vitro when compared to that observed in the absence of the nanoparticles described herein.

For purposes of the present invention, “inhibiting” or “downregulating” shall be understood to mean that the expression of a target gene, or level of RNAs or equivalent RNAs encoding one or more protein subunits, or activity of one or more protein subunits, such as ErbB3, HIF-1a, Survivin and BCL2, is reduced when compared to that observed in the absence of the nanoparticles described herein.

In one preferred embodiment, a target gene includes, for example, but is not limited to, oncogenes, pro-angiogenesis pathway genes, pro-cell proliferation pathway genes, viral infectious agent genes, and pro-inflammatory pathway genes.

Preferably, gene expression of a target gene is inhibited in cancer cells or tissues, for example, brain, breast, colorectal, gastric, lung, mouth, pancreatic, prostate, skin or cervical cancer cells. The cancer cells or tissues can be from one or more of the following: solid tumors, lymphomas, small cell lung cancer, acute lymphocytic leukemia (ALL), pancreatic cancer, glioblastoma, ovarian cancer, gastric cancer, breast cancer, colorectal cancer, prostate cancer, cervical cancer, brain tumors, KB cancer, lung cancer, colon cancer, epidermal cancer, etc.

In one particular embodiment, the nanoparticles according to the methods described herein include, for example, antisense bcl-2 oligonucleotides, antisense HIF-1a oligonucleotides, antisense Survivin oligonucleotides and antisense ErbB3 oligonucleotides.

The therapy contemplated herein uses nucleic acids encapsulated in the aforementioned nanoparticle. In one embodiment, therapeutic nucleotides containing eight or more consecutive antisense nucleotides can be employed in the treatment.

In one particular treatment, the nanoparticles including oligonucleotides (SEQ ID NO. 1, SEQ ID NOs: 2 & 3, SEQ ID NO: 4, SEQ ID NO: 5, and SEQ ID NO: 6) can be used.

Alternatively, there are also provided methods of treating a mammal. The methods include administering an effective amount of a pharmaceutical composition containing a nanoparticle described herein to a patient in need thereof. The efficacy of the methods would depend upon efficacy of the nucleic acids for the condition being treated. The present invention provides methods of treatment for various medical conditions in mammals. The methods include administering, to the mammal in need of such treatment, an effective amount of a nanoparticle containing encapsulated therapeutic nucleic acids. The nanoparticles described herein are useful for, among other things, treating diseases such as (but not limited to) cancer, inflammatory disease, and autoimmune disease.

In one embodiment, there are also provided methods of treating a patient having a malignancy or cancer, comprising administering an effective amount of a pharmaceutical composition containing the nanoparticle described herein to a patient in need thereof. The cancer being treated can be one or more of the following: solid tumors, lymphomas, small cell lung cancer, acute lymphocytic leukemia (ALL), pancreatic cancer, glioblastoma, ovarian cancer, gastric cancers, colorectal cancer, prostate cancer, cervical cancer, brain tumors, KB cancer, lung cancer, colon cancer, epidermal cancer, etc. The nanoparticles are useful for treating neoplastic disease, reducing tumor burden, preventing metastasis of neoplasms and preventing recurrences of tumor/neoplastic growths in mammals by downregulating gene expression of a target gene.

The nanoparticles are useful for treating neoplastic disease, reducing tumor burden, preventing metastasis of neoplasms and preventing recurrences of tumor/neoplastic growths in mammals by downregulating gene expression of a target gene. For example, the nanoparticles are useful in the treatment of metastatic disease (i.e. cancer with metastasis into the liver).

In yet another aspect, the present invention provides methods of inhibiting the growth or proliferation of cancer cells in vivo or in vitro. The methods include contacting cancer cells with the nanopaticle described herein. In one embodiment, the present invention provides methods of inhibiting the growth of cancer in vivo or in vitro wherein the cells express ErbB3 gene. In another embodiment, the present invention provides a means to deliver an antisense oligonucleotide such as an antisense ErbB3 LNA oligonucleotide inside a cancer cell in which the antisense oligonucleotide can enter the nucleus and bind to ErbB3 mRNA. As a consequence, target gene expression such as the ErbB3 expression is inhibited, which inhibits the growth of the cancer cells. Alternatively, the present invention provides methods of modulating apoptosis in cancer cells. The method includes contacting cells with the nanoparticle described herein.

In yet another aspect, there are also provided methods of increasing the sensitivity of cancer cells or tissues to chemotherapeutic agents in vivo or in vitro. In one particular aspect, the methods include introducing an oligonucleotide (e.g. antisense oligonucleotides including LNA) encapsulated in the nanoparticle described herein to cancer cells to reduce gene (e.g., survivin, HIF-1α or ErbB3) expression in the cancer cells or tissues, wherein the antisense oligonucleotide binds to mRNA and reduces gene expression.

In yet another aspect, there are provided methods of killing tumor cells in vivo or in vitro. The methods include introducing the nanoparticles described herein to tumor cells to reduce gene expression such as ErbB3 gene and contacting the tumor cells with an amount of at least one chemotherapeutic agent sufficient to kill a portion of the tumor cells. Thus, the portion of tumor cells killed can be greater than the portion which would have been killed by the same amount of the chemotherapeutic agent in the absence of the nanoparticles described herein.

In a further aspect of the invention, a chemotherapeutic agent can be used in combination, simultaneously or sequentially, in the methods employing the nanoparticles described herein. The nanoparticles described herein can be administered prior to or concurrently with the chemotherapeutic agent, or after the administration of the chemotherapeutic agent.

Still further aspects include combining the compound of the present invention described herein with other anticancer therapies for synergistic or additive benefit.

Alternatively, the nanoparticle composition described herein can be used to deliver a pharmaceutically active agent, preferably having a negative charge or a neutral charge to a mammal. The nanoparticle encapsulating pharmaceutically active agents/compounds can be administered to a mammal in need thereof. The pharmaceutically active agents/compounds include small molecular weight molecules. Typically, the pharmaceutically active agents have a molecular weight of less than about 1,500 daltons (i.e., less than 1,000 daltons).

In a further embodiment, the compounds described herein can be used to deliver nucleic acids, a pharmaceutically active agent, or in a combination thereof.

In yet a further embodiment, the nanoparticle associated with the treatment can contain a mixture of one or more therapeutic nucleic acids (either the same or different, for example, the same or different oligonucleotides containing LNA) and pharmaceutically active agents for synergistic application.

### F. Pharmaceutical Compositions/Formulations of Nanoparticles

Pharmaceutical compositions/formulations including the nanoparticles described herein may be formulated in conjunction with one or more physiologically acceptable carriers comprising excipients and auxiliaries which facilitate processing of the active compounds into preparations which can be used pharmaceutically. Proper formulation is dependent upon the route of administration chosen, i.e., whether local or systemic treatment is treated.

Suitable forms, in part, depend upon the use or the route of entry, for example oral, transdermal, or injection. Factors for considerations known in the art for preparing proper formulations include, but are not limited to, toxicity and any disadvantages that would prevent the composition or formulation from exerting its effect.

Administration of pharmaceutical compositions of nanoparticles described herein may be oral, pulmonary, topical (e.g., epidermal, transdermal, ophthalmic and mucous membranes including vaginal and rectal delivery) or parenteral including intravenous, intraarterial, subcutaneous, intraperitoneal or intramuscular injection or infusion.

In one preferred embodiment, the nanoparticles containing therapeutic oligonucleotides are administered intravenously (i.v.), intraperitoneally (i.p.) or as a bolus injection. Parenteral routes are preferred in many aspects of the invention.

For injection, including, without limitation, intravenous, intramuscular and subcutaneous injection, the nanoparticles of the invention may be formulated in aqueous solutions, preferably in physiologically compatible buffers such as physiological saline buffer or polar solvents including, without limitation, a pyrrolidone or dimethylsulfoxide.

The nanoparticles may also be formulated for bolus injection or for continuous infusion. Formulations for injection may be presented in unit dosage form, e.g., in ampoules or in multi-dose containers. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water soluble form. Aqueous injection suspensions may contain substances that modulate the viscosity of the suspension, such as sodium carboxymethyl cellulose, sorbitol, or dextran. Optionally, the suspension may also contain suitable stabilizers and/or agents that increase the concentration of the nanoparticles in the solution. Alternatively, the nanoparticles may be in powder form for constitution with a suitable vehicle, e.g., sterile, pyrogen-free water, before use.

For oral administration, the nanoparticles described herein can be formulated by combining the nanoparticles with pharmaceutically acceptable carriers well-known in the art. Such carriers enable the nanoparticles of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars (for example, lactose, sucrose, mannitol, or sorbitol), cellulose preparations such as maize starch, wheat starch, rice starch and potato starch and other materials such as gelatin, gum tragacanth, methyl cellulose, hydroxypropylmethylcellulose, sodium carboxymethylcellulose, and/or polyvinylpyrrolidone (PVP). If desired, disintegrating agents may be added, such as cross-linked polyvinyl pyrrolidone, agar, or alginic acid. A salt such as sodium alginate may also be used.

For administration by inhalation, the nanoparticles of the present invention can conveniently be delivered in the form of an aerosol spray using a pressurized pack or a nebulizer and a suitable propellant.

The nanoparticles may also be formulated in rectal compositions such as suppositories or retention enemas, using, e.g., conventional suppository bases such as cocoa butter or other glycerides.

In addition to the formulations described previously, the nanoparticles may also be formulated as depot preparations. Such long acting formulations may be administered by implantation (for example, subcutaneously or intramuscularly) or by intramuscular injection. A nanoparticle of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Additionally, the nanoparticles may be delivered using a sustained-release system, such as semi-permeable matrices of solid hydrophobic polymers containing the nanoparticles. Various sustained-release materials have been established and are well known by those skilled in the art.

In addition, antioxidants and suspending agents can be used in the pharmaceutical compositions of the nanoparticles described herein.

### G. Dosages

Determination of doses adequate to inhibit the expression of one or more preselected genes, such as a therapeutically effective amount in the clinical context, is well within the capability of those skilled in the art, especially in light of the disclosure herein.

For any therapeutic nucleic acids used in the methods of the invention, the therapeutically effective amount can be estimated initially from in vitro assays. Then, the dosage can be formulated for use in animal models so as to achieve a circulating concentration range that includes the effective dosage. Such information can then be used to more accurately determine dosages useful in patients.

The amount of the pharmaceutical composition that is administered will depend upon the potency of the nucleic acids included therein. Generally, the amount of the nanoparticles containing nucleic acids used in the treatment is that amount which effectively achieves the desired therapeutic result in mammals. Naturally, the dosages of the various nanoparticles will vary somewhat depending upon the nucleic acids (or pharmaceutically active agents) encapsulated therein (oligonucleotides such as antisense LNA molecules). In addition, the dosage, of course, can vary depending upon the dosage form and route of administration. In general, however, the nucleic acids encapsulated in the nanoparticles described herein can be administered in amounts ranging from about 0.1 mg/kg/dose to about 1 g/kg/dose, preferably from about 1 to about 500 mg/kg/dose and more preferably from 1 to about 100 mg/kg/dose (i.e., from about 2 to about 60 mg/kg/dose). The antisense oligonucleotide administered in the therapy can range in an amount of from about 4 to about 25 mg/kg/dose. For example, the treatment protocol includes administering an antisense oligonucleotide ranging from about 0.1 mg/kg/week to about 1 g/kg/week, preferably from about 1 to about 500 mg/kg/week and more preferably from 1 to about 100 mg/kg/week (i.e., from about 2 to about 60 mg/kg/week).

In one embodiment, the protocol includes administering an antisense oligonucleotide in an amount of about 4 to about 18 mg/kg/dose weekly, or about 4 to about 9.5 mg/kg/dose weekly.

In one particular embodiment, the treatment protocol includes an antisense oligonucleotide in an amount of about 4 to about 18 mg/kg/dose weekly for 3 weeks in a six week cycle (i.e. about 8 mg/kg/dose). Another particular embodiment includes about 4 to about 9.5 mg/kg/dose weekly (i.e., about 8 or 4.1 mg/kg/dose).

The range set forth above is illustrative and those skilled in the art will determine the optimal dosing based on clinical experience and the treatment indication. Moreover, the exact formulation, route of administration and dosage can be selected by the individual physician in view of the patient's condition. Additionally, toxicity and therapeutic efficacy of the compounds described herein can be determined by standard pharmaceutical procedures in cell cultures or experimental animals using methods well-known in the art.

Alternatively, an amount of from about 0.1 mg to about 140 mg/kg/day (0.1 to 100 mg/kg/day) can be used in the treatment depending on potency of the nucleic acids. Dosage unit forms generally range from about 1 mg to about 500 mg of an active agent, oligonucleotides.

In one embodiment, the treatment of the present invention includes administering the oligonucleotide encapsulated within the nanoparticles described herein in an amount of from about 0.1 to about 50 mg/kg/dose, such as from about 0.5 to about 45 mg/kg/dose (e.g. either in a single or multiple dose regime) to a mammal.

Alternatively, the delivery of the oligonucleotide encapsulated within the nanoparticles described herein includes contacting a concentration of oligoncleotides of from about 0.1 to about 1000 nM, preferably from about 10 to about 1500 nM (i.e. from about 30 to about 1000 nM) with tumor cells or tissues in vivo, ex vivo or in vitro.

The compositions may be administered once daily or divided into multiple doses which can be given as part of a multi-week treatment protocol. The precise dose will depend on the stage and severity of the condition, the susceptibility of the disease such as tumor to the nucleic acids, and the individual characteristics of the patient being treated, as will be appreciated by one of ordinary skill in the art.

In all aspects of the invention where nanoparticles are administered, the dosage amount mentioned is based on the amount of oligonucleotide molecules rather than the amount of nanoparticles administered.

It is contemplated that the treatment will be given for one or more days until the desired clinical result is obtained. The exact amount, frequency and period of administration of the nanoparticles encapsulating therapeutic nucleic acids (or pharmaceutically active agents) will vary, of course, depending upon the sex, age and medical condition of the patent as well as the severity of the disease as determined by the attending clinician.

Still further aspects include combining the nanoparticles of the present invention described herein with other anticancer therapies for synergistic or additive benefit.

### EXAMPLES

The following examples serve to provide further appreciation of the invention but are not meant in any way to restrict the effective scope of the invention.

In the examples, all synthesis reactions are run under an atmosphere of dry nitrogen or argon. N-(3-aminopropyl)-1,3-propanediamine, BOC-ON, ethylene oxide, LiOCl₄, cholesterol and 1H-pyrazole-1-carboxamidnie.HCl were purchased from Aldrich. All other reagents and solvents were used without further purification. An LNA-containing oligonucleotides such as Oligo-1 targeting survivin gene, Oligo-2 targeting ErbB3 gene and Oligo-3 (scrambled Oligo-2) were prepared in house and their sequences are described in Table 4. The internucleoside linkage in the oligonucleotides includes phosphorothioate, ^{m}C represents methylated cytosine, and the upper case letters indicate LNA.

TABLE 4

| LNA Oligo | Sequence |
|---|---|
| Oligo-1 | 5′-^{m}CT^{m}CAatccatgg^{m}CAGc-3′ |
| (SEQ ID NO: 1) | |
| Oligo-2 | 5′-TAGcctgtcactt^{m}CT^{m}V-3′ |
| (SEQ ID NO: 6) | |
| Oligo-3 | 5′-TAGcttgtcccat^{m}CT^{m}C-3 |
| (SEQ ID NO: 7) | |

The following abbreviations are used throughout the examples, such as LNA (Locked nucleic acid oligonucleotide), BACC (2-[N,N′-di(2-guanidiniumpropyl)]aminoethylcholesteryl-carbonate), 2-(Boc-oxyimino)-2-phenylacetatonitrile (BOC-ON), Chol (cholesterol), DIEA (diisopropylethylamine), DMAP (4-N,N-dimethylamino-pyridine), DOPE (L-a-dioleoyl phosphatidylethanolamine, Avanti Polar Lipids, USA or NOF, Japan), DLS (Dynamic Light Scaterring), DSPC (1,2-distearoyl-sn-glycero-3-phosphocholine) (NOF, Japan), DSPE-PEG (1,2-distearoyl-sn-glycero-3-phosphoethanolamine-N-(polyethylene glycol)2000 ammonium salt or sodium salt, Avanti Polar Lipids, USA and NOF, Japan), KD (knowndown), EPC (egg phosphatidylcholine, Avanti Polar Lipids, USA) and C16mPEG-Ceramide (N-palmitoyl-sphingosine-1-[succinyl(methoxypolyethylene glycol)2000, Avanti Polar Lipids, USA). Other abbreviations such as FAM (6-carboxyfluorescein), FBS (fetal bovine serum), GAPDH (glyceraldehyde-3-phosphate dehydrogenase), DMEM (Dulbecco's Modified Eagle's Medium), MEM (Modified Eagle's Medium), TEAA (tetraethylammonium acetate), TFA (trifluoroacetic acid), RT-qPCR (reverse transcription-quantitative polymerase chain reaction) were also used.

### Example 1

### General NMR Method

¹H NMR spectra were obtained at 300 MHz and ¹³C NMR spectra at 75.46 MHz using a Varian Mercury 300 NMR spectrometer and deuterated chloroform as the solvents unless otherwise specified. Chemical shifts (δ) are reported in parts per million (ppm) downfield from tetramethylsilane (TMS).

### Example 2

### General mRNA Down-Regulation Procedure

Cells are maintained in a complete medium (F-12K or DMEM, supplemented with 10% FBS). A 12 well plate containing 2.5×10⁵ cells in each well is incubated overnight at 37° C. The cells are washed once with Opti-MEM® and 400 μL of Opti-MEM® is added to each well. Then, the cells are treated with a nanoparticle solution encapsulating nucleic acids or a solution of free nucleic acids without the nanoparticles (naked oligonucleotides) as a control. The cells are incubated for 4 hours, followed by addition of 600 μL of media per well, and incubation for 24 hours. After 24 hours of the treatment, the intracellular mRNA levels of a target gene such as human ErbB3, and a housekeeping gene such as GAPDH are measured by RT-qPCR. The expression levels of mRNA are normalized to that of GAPDH.

### Example 3

### Preparation of Compound 1

To a solution of 2,2′-(ethane-1,2-diylbis(oxy))diethanamine (101.2 g, 683 mmol) in 250 mL of anhydrous dichloromethane (DCM) and 200 mL of THF was added a solution of di-tert-butyl dicarbonate (59.6 g, 273 mmol) in 150 mL of anhydrous DCM at 0° C. slowly over a period of 1.5 hours. The mixture was stirred for 16 hours at room temperature. The solvent was removed and the residue was taken into 300 mL of water and extracted into DCM (2×300 mL) The organic layers were combined and extracted with 0.5N HCl (2×250 mL). The aqueous layer was then basified with a 4 N sodium hydroxide solution to pH 8 and extracted with DCM (2×300 mL). The organic layers were combined and dried over anhydrous magnesium sulfate, filtered, concentrated and dried under vacuum at 40° C. to yield 28.5 g (yield 42%) of product: ¹³C NMR d 155.43, 78.42, 73.05, 69.74, 41.37, 39.92, 28.06.

### Example 4

### Preparation of Compound 2

To a solution of compound 1 (3.52 g, 14.2 mmol) in 70 mL of anhydrous DCM was added DIEA (2.48 mL, 14.2 mmol), followed by cholestery chloroformate (5.8 g, 12.9 mmol). The reaction mixture was stirred at room temperature for 2.5 hours, followed by 0.5N HCl (60 mL). The product was extracted into DCM (2×60 mL). The combined organic layers were dried over anhydrous magnesium sulfate, filtered and concentrated. The solids obtained were dried under vacuum at 35° C. to yield 8.05 g (yield 94%) of product: ¹³C NMR d 155.99, 155.80, 139.71, 122.36, 79.26, 76.57, 74.35, 70.29, 70.2, 56.71, 56.16, 50.04, 42.35, 40.74, 40.42, 39.78, 39.57, 38.62, 37.05, 36.61, 36.22, 35.85, 31.94, 28.48, 28.29, 28.23, 28.07, 24.35, 23.91, 22.88, 22.63, 21.12, 19.43, 18.80, 11.95.

### Example 5

### Preparation of Compound 3

To a solution of compound 2 (8.05 g, 12.2 mmol) in 55 mL of anhydrous DCM was added 24 mL of TFA at 0° C. The reaction mixture was stirred at room temperature for 1 hour. After the reaction was complete, the solvent was removed to dryness to yield 9.15 g (yield quant.) as a TFA salt. This compound was used without further purification: ¹³C NMR d 161.31, 160.77, 160.24, 159.72, 157.90, 139.17, 122.82, 120.84, 117.04, 113.24, 109.45, 70.05, 69.97, 66.14, 66.08, 56.72, 56.22, 50.06, 42.36, 40.79, 40.09, 39.80, 39.60, 38.33, 36.90, 36.56, 36.27, 35.88, 31.92, 28.31, 28.07, 27.98, 24.35, 23.95, 22.84, 22.59, 21.11, 19.25, 18.78, 14.51, 11.92.

### Example 6

### Preparation of Compound 4

To a solution of compound 3 (9.15 g, 13.6 mmol) in 100 mL of anhydrous DCM at 0° C. was added Boc-Lys(Boc)-OH (10.7 g, 20.4 mmol) followed by DMAP (2.5 g, 20.4 mmol) and EDC (3.92 g, 20.4 mmol). The mixture was stirred overnight at room temperature. The reaction was diluted with 100 mL of DCM, washed with 0.5N NaHCO₃ (2×70 mL) and 0.1N HCl (2×70 mL). The organic layer was dried over anhydrous magnesium sulfate, filtered and concentrated. The crude material was purified by silica gel column chromatography using a mixture of DCM/methanol (9:1, v/v) to yield 5.1 g (yield 42%) of product: ¹³C NMR d 171.90, 156.04, 155.84, 155.43, 139.54, 122.30, 79.76, 78.89, 74.25, 70.23, 70.09, 69.59, 56.59, 56.05, 54.37, 53.36, 49.94, 42.25, 40.63, 40.01, 39.67, 39.46, 39.19, 38.53, 36.94, 36.50, 36.13, 35.74, 32.31, 31.83, 29.64, 28.44, 28.33, 28.19, 28.15, 27.96, 24.26, 23.80, 22.81, 22.65, 22.55, 21.02, 19.31, 18.70, 11.86.

### Example 7

### Preparation of Compound 5

To a solution of compound 4 (5.1 g, 5.7 mmol) in 35 mL of anhydrous DCM was added 15 mL of TFA at 0° C. The reaction mixture was stirred at room temperature for 1.5 hours. The mixture was diluted with 50 mL of DCM and saturated NaHCO₃ solution was slowly added until the aqueous layer attained pH ˜5. The organic layer was separated and dried over anhydrous MgSO₄, filtered, concentrated and dried to yield 3.8 g (73%) of product as TFA salt: ¹³C NMR d 169.29, 156.33, 139.70, 122.40, 76.57, 74.35, 70.06, 56.72, 56.24, 50.03, 42.37, 39.79, 39.56, 38.69, 37.03, 36.61, 36.28, 35.90, 31.94, 28.29, 28.08, 24.38, 24.02, 22.90, 22.64, 21.17, 19.47, 18.83, 11.98,

### Example 8

### Preparation of Compound 6

To a solution of compound 5 (1.3 g, 1.88) in 13 mL of anhydrous chloroform was added 1H-pyrazole-1-carboxamidine HCl (1.10 g, 7.5 mmol), followed by DIEA (1.31 mL, 7.5 mmol, d 0.74) at room temperature. The reaction was refluxed for 16 hours. The solution was cooled to room temperature and precipitated by adding 15 mL acetonitrile. The solids were isolated with a centrifuge. The isolated solids were redissolved in 14 mL of water/ACN (1:1, v/v). After dissolution, 14 mL of ACN was added to precipitate solids. The solids were centrifuged and dried to yield 950 mg (66%) of product: ¹³C NMR d 171.06, 157.05, 156.43, 139.62, 122.39, 74.42, 70.03, 56.69, 56.24, 49.99, 42.32, 40.64, 39.76, 39.53, 38.63, 37.05, 36.57, 36.25, 35.87, 31.92, 28.26, 28.04, 24.35, 23.99, 22.87, 22.61, 21.13, 19.44, 18.80, 11.97.

### Example 9

### Preparation of Compound 7

To a solution of cholesterol (14.2 g, 36.8 mmol) in 140 mL of anhydrous DCM at 0° C. was added 2-(2-(2-Boc-aminoethoxy)ethoxy)acetic acid (5.1 g, 18.4 mmol) followed by DMAP (6.7 g, 54.8 mmol) and EDC (7.1 g, 36.8 mmol). The mixture was stirred at room temperature for 18 hours. The reaction mixture was diluted with 50 mL DCM, washed with 0.5N NaHCO₃ (2×80 mL) and 0.1N HCl (2×80 mL). The combined organic layers were dried over anhydrous magnesium sulfate, filtered and concentrated. The crude material was purified by silica gel column chromatography using a mixture of 25% EtOAc/hexanes to yield 8.5 g (72%) of product: ¹³C NMR d 169.57, 155.78, 139.16, 122.75, 79.05, 74.62, 70.76, 70.29, 70.24, 68.73, 56.64, 56.11, 49.99, 42.31, 40.39, 39.72, 39.52, 38.07, 36.92, 36.56, 36.19, 35.78, 31.91, 31.86, 28.43, 28.23, 28.02, 27.76, 24.30, 23.86, 22.85, 22.59, 21.05, 19.33, 18.75, 11.90.

### Example 10

### Preparation of Compound 8

To a solution of compound 7 (8.5 g, 13.4 mmol) in 80 mL of anhydrous DCM was added 20 mL of TFA at 0° C. The reaction was stirred at room temperature for 1.5 hours. After the reaction was complete, the solvent was removed to dryness to yield 10 g (yield quant.) of product as TFA salt. This compound was used without further purification: ¹³C NMR d 171.10, 160.78, 160.24, 138.74, 123.22, 116.93, 113.14, 77.42, 76.23, 70.54, 69.80, 68.21, 66.62, 56.66, 56.15, 49.98, 42.35, 40.10, 39.74, 39.56, 37.85, 36.78, 36.54, 36.23, 35.85, 31.88, 28.29, 28.07, 27.57, 24.34, 23.91, 22.87, 22.61, 21.08, 19.28, 18.78, 11.93.

### Example 11

### Preparation of Compound 9

To a solution of compound 8 (10 g, 15.5 mmol) in 100 mL of anhydrous DCM at 0° C. was added Boc-Lys(Boc)-OH (20.4 g, 38.8 mmol) followed by DMAP (5.6 g, 38.8 mmol) and EDC (7.27 g, 38.8 mmol). The mixture was stirred overnight at room temperature. The reaction was diluted with 100 mL DCM, washed with 0.5N NaHCO₃ (2×80 mL) and 0.1N HCl (2×80 mL). The organic layer was dried over anhydrous magnesium sulfate, filtered and concentrated. The crude material was purified by silica gel column chromatography using a mixture of DCM/methanol (9:1, v/v) to yield 6.2 g (47%) of product: ¹³C NMR: d 172.00, 169.82, 155.93, 139.15, 122.88, 78.98, 77.42, 74.92, 74.85, 70.90, 70.20, 70.10, 69.69, 68.78, 68.64, 68.53, 56.67, 56.12, 54.35, 49.99, 42.34, 40.16, 39.85, 39.75, 39.55, 39.27, 38.08, 36.94, 36.60, 36.21, 35.83, 32.64, 31.94, 31.89, 29.68, 28.51, 28.42, 28.26, 28.07, 27.99, 27.91, 27.82, 24.33, 23.99, 23.87, 22.88, 22.68, 22.62, 21.08, 19.36, 18.78, 11.93.

### Example 12

### Preparation of Compound 10

To a solution of compound 9 (6.2 g, 7.2 mmol) in 50 mL of anhydrous DCM was added 20 mL of TFA at 0° C. The reaction was stirred at room temperature for 1.5 hours. The reaction mixture was diluted with 60 mL of DCM and saturated NaHCO₃ solution was slowly added until the aqueous layer attained pH ˜5. The organic layer was separated and dried over anhydrous MgSO₄, filtered, concentrated and dried to yield 4.8 g (86%) of product as TFA salt: ¹³C NMR d 176.88, 171.39, 162.97, 140.68, 123.68, 116.13, 75.85, 71.82, 71.21, 71.03, 70.40, 69.67, 69.35, 64.65, 58.10, 57.69, 55.66, 51.54, 43.57, 41.17, 40.79, 40.62, 40.17, 39.16, 38.28, 37.77, 37.54, 37.25, 35.35, 33.21, 33.12, 29.48, 29.22, 28.84, 28.76, 25.47, 25.33, 25.25, 2152, 23.44, 23.21, 22.28, 19.96, 19.58, 12.66.

### Example 13

### Preparation of Compound 11

To a solution of compound 10 (1 g, 1.48 mmol) in 12 mL of anhydrous chloroform was added 1H-pyrazole-1-carboxamidine HCl (0.87 g, 5.9 mmol) followed by DMA (1.03 mL, 5.9 mmol, d 0.74) at room temperature. The reaction was refluxed for 16 hours. The solution was cooled to room temperature. The mixture was precipitated with 15 mL of ACN and crude solids were isolated with centrifuge. The solids were redissolved in 14 mL of water/ACN (1:1) solution. After dissolution, 14 mL ACN was added to precipitate solids. The solids were centrifuged and dried to yield 400 mg (36%) of product: ¹³C NMR d 171.18, 170.05, 157.01, 139.15, 122.85, 74.91, 70.60, 69.73, 69.10, 68.42, 56.66, 56.24, 55.03, 49.98, 42.36, 41.34, 39.75, 39.55, 38.06, 36.93, 36.61, 36.27, 35.91, 31.91, 28.30, 28.09, 27.80, 24.36, 24.04, 22.90, 22.65, 21.13, 19.40, 18.82, 11.99.

### Example 14

### Preparation of Compound 12, Cholesterol-Lys(Boc)₂

To a solution of cholesterol (6.0 g, 15.5 mmol) in 100 mL anhydrous DCM at 0° C. is added Boc-Lys(Boc)-OH (20.4 g, 38.8 mmol) followed by DMAP (5.6 g, 38.8 mmol) and EDC (7.27 g, 38.8 mmol). The mixture is stirred overnight at room temperature. The reaction is diluted with 100 mL DCM, washed with 0.5N NaHCO₃ (2×80 mL) and 0.1N HCl (2×80 mL). The organic layer is dried over anhydrous magnesium sulfate, filtered and concentrated. The crude material is purified by silica gel column chromatography using a mixture of DCM/methanol (9:1, v/v) to yield product.

### Example 15

### Preparation of Compound 13, Cholesterol-Lys(NH₂)₂

To a solution of compound 12 (9.6 g, 13.4 mmol) in 80 mL anhydrous DCM is added 20 mL TFA at 0° C. The reaction is stirred at room temperature for 1.5 hours. After completion of the reaction, the solvent is removed to dryness to yield product in quantitative yield as TFA salt. This compound is used without further purification.

### Example 16

### Preparation of Compound 14, Cholesterol-Lys[NH—C(═NH)(NH₂)₂]

To a solution of compound 13 (762 mg, 1.48 mmol) in 12 mL of anhydrous chloroform is added 1H-pyrazole-1-carboxamidine HCl (0.87 g, 5.9 mmol) followed by DIEA (1.03 mL, 5.9 mmol, d 0.74) at room temperature. The reaction is refluxed for 16 hours. The solution is cooled to room temperature. The mixture is precipitated with 15 mL of ACN and crude solids are isolated with centrifuge. The solids are redissolved in 14 mL of water/ACN (1:1) solution. After dissolution, 14 mL of ACN is added to precipitate solids. The solids are centrifuged and dried to yield product.

### Example 17

### Preparation of Nanoparticles

In this example, nanoparticle compositions encapsulating various nucleic acids such as LNA-containing oligonucleotides are prepared. For example, compound 6, DOPE, Chol, DSPE-PEG and ConPEG-Ceramide are mixed at a molar ratio of 18:60:20:1:1 in 10 mL of 90% ethanol (total lipid 30 μmole). LNA oligonucleotides (0.4 μmole) are dissolved in 10 mL of 20 mM Tris buffer (pH 7.4-7.6). After being heated to 37° C., the two solutions are mixed together through a duel syringe pump and the mixed solution is subsequently diluted with 20 mL of 20 mM Tris buffer (300 mM NaCl, pH 7.4-7.6). The mixture is incubated at 37° C. for 30 minutes and dialyzed in 10 mM PBS buffer (138 mM NaCl, 2.7 mM KCl, pH 7.4). Stable particles are obtained after the removal of ethanol from the mixture by dialysis. The nanoparticle solution is concentrated by centrifugation. The nanoparticle solution is transferred into a 15 mL centrifugal filter device (Amicon Ultra-15, Millipore, USA). Centrifuge speed is at 3,000 rpm and temperature is at 4° C. during centrifugation. The concentrated suspension is collected after a given time and is sterilized by filtration through a 0.22 μm syringe filter (Millex-GV, Millipore, USA).

The diameter and polydispersity of nanoparticle are measured at 25° in water (Sigma) as a medium on a Plus 90 Particle Size Analyzer Dynamic Light Scattering Instrument (Brookhaven, N.Y.).

Encapsulation efficiency of LNA oligonucleotides is determined by UV-VIS (Agilent 8453). The background UV-vis spectrum is obtained by scanning solution, which is a mixed solution composed of PBS buffer saline (250 μL), methanol (625 μL) and chloroform (250 μL). In order to determine the encapsulated nucleic acids concentration, methanol (625 μL) and chloroform (250 μL) are added to PBS buffer saline nanoparticle suspension (250 μL). After mixing, a clear solution is obtained and this solution is sonicated for 2 minutes before measuring absorbance at 260 nm. The encapsulated nucleic acid concentration and loading efficiency is calculated according to equations (1) and (2):

Cₑₙ(μg/ml)=A₂₆₀×OD₂₆₀ unit (μg/mL)×dilution factor (μL/μL) (1)

where the dilution factor is given by the assay volume (μL) divided by the sample stock volume (μL).

Encapsulation efficiency (%)=[Cₑₙ/Cᵢₙᵢₜᵢₐₗ]×100 (2)

where Cₑₙ is the nucleic acid (i.e., LNA oligonucleotide) concentration encapsulated in nanoparticle suspension after purification, and Cᵢₙᵢₜᵢₐₗ is the initial nucleic acid (LNA oligonucleotide) concentration before the formation of the nanoparticle suspension. Examples of various nanoparticle compositions are summarized in Tables 5 and 6.

TABLE 5

| Sample | | | |
|---|---|---|---|
| No. | Nanoparticle Composition | Molar Ratio | Oligo |
| 1 | Compd 6: DOPE: DSPC: | 15:15:20:40:10 | Oligo-1 |
| | Chol: PEG-DSPE | | |
| 2 | Compd 6: DOPE: DSPC: | 15:5:20:50:10 | Oligo-1 |
| | Chol: PEG-DSPE | | |
| 3 | Compd 6: DOPE: DSPC: | 25:15:20:30:10 | Oligo-1 |
| | Chol: PEG-DSPE | | |
| 4 | Compd 6: EPC: Chol: PEG-DSPE | 20:47:30:3 | Oligo-1 |
| 5 | Compd 6: DOPE: Chol: PEG-DSPE | 17:60:20:3 | Oligo-1 |
| 6 | Compd 6: DOPE: PEG-DSPE | 20:78:2 | Oligo-1 |
| 7 | Compd 6: DOPE: Chol: | 17:60:20:3 | Oligo-2 |
| | C16mPEG-Ceramide | | |
| 8 | Compd 6: DOPE: Chol: PEG- | 18:60:20:1:1 | Oligo-2 |
| | DSPE: C16mPEG-Ceramide | | |

TABLE 6

| Sample | Nanoparticle | | |
|---|---|---|---|
| No. | Composition | Molar Ratio | Oligo |
| NP1 | Compd 6: DOPE: Chol: | 18:60:20:1:1 | Oligo-2 |
| | PEG-DSPE: | | |
| | C16mPEG-Ceramide | | |
| NP2 | Compd 6: DOPE: Chol: | 18:60:20:1:1 | Scrambled |
| | PEG-DSPE: | | Oligo-2 |
| | C16mPEG-Ceramide | | (=Oligo-3) |
| NP3 | Compd 6: DOPE: Chol: | 18:60:20:1:1 | FAM- |
| | PEG-DSPE: | | Oligo-2 |
| | C16mPEG-Ceramide | | |
| NP4 | Compd 6: DOPE: Chol: | 18:60:20:1:1 | none |
| | PEG-DSPE: | | |
| | C16mPEG-Ceramide | | |

### Example 18

### Nanoparticle Stability

Nanoparticle stability is defined as their capability to retain the structural integrity in PBS buffer at 4° C. over time. The colloidal stability of nanoparticles is evaluated by monitoring changes in the mean diameter over time. Nanoparticles prepared by Sample No. NP1 in Table 6 are dispersed in 10 mM PBS buffer (138 mM NaCl, 2.7 mM KCl, pH 7.4) and stored at 4° C. At a given time point, about 20-50 μL of the nanoparticle suspension is taken and diluted with pure water up to 2 mL. The sizes of nanoparticles are measured by DLS at 25° C.

### Example 19

### In Vitro Nanoparticle Cellular Uptake

The efficiency of cellular uptake of nucleic acids (LNA oligonucleotide Oilgo-2) encapsulated in the nanoparticle described herein is evaluated in human cancer cells such as prostate cancer cells (15PC3 cell line). Nanoparticles of Sample NP3 are prepared using the method described in Example 16. LNA oligonucleotides (Oligo-2) are labeled with FAM for fluorescent microscopy studies.

The nanoparticles are evaluated in the 15PC3 cell line. The cells are maintained in a complete medium (DMEM, supplemented with 10% FBS). A 12 well plate containing 2.5×10⁵ cells in each well is incubated overnight at 37° C. The cells are washed once with Opti-MEM and 400 mL of Opti-MEM is added to each well. Then, the cells are treated with a nanoparticle solution of Sample No. NP3 (200 nM) encapsulating nucleic acids (FAM-modified Oligo 2) or a solution of free nucleic acids without the nanoparticles (naked FAM-modified Oligo 2) as a control. The cells are incubated for 24 hours at 37° C. The cells are washed with PBS five times, and then stained with 300 mL of Hoechst solution (2 mg/mL) per well for 30 minutes, followed by washing with PBS 5 times. The cells are fixed with pre-cooled (−20° C.) 70% EtOH at −20° C. for 20 minutes: The cells are inspected under fluorescent microscope to evaluate the efficiency of cellular uptake of nucleic acids encapsulated within the nanoparticle described herein.

### Example 20

### In Vitro Efficacy of Nanoparticles on mRNA Down-Regulation in Human Epidermal Cancer Cells

The efficacy of the nanoparticles described herein is evaluated in human epidermal cancer cells (A431 cell line). The A431 cells overexpress epidermal growth factor receptors (EGFR). The cells are treated with nanoparticles encapsulating antisense ErbB3 oligonucleotides (Sample NP5). The cells are also treated with nanoparticles encapsulating oligonucleotides with a scrambled sequence (Sample No. NP6) or empty placebo nanoparticles (Sample No. NP7) as a control. The nanoparticles are prepared using the method described in Example 17 (Table 7). The in vitro efficacy of each of the nanoparticles on downregulation of ErbB3 expression is measured by the procedures described in Example 2.

TABLE 7

| Sample | Nanoparticle | | |
|---|---|---|---|
| No. | Composition | Molar Ratio | Oligo |
| NP5 | Compd 6: DOPE: Chol: | 18:60:20:1:1 | Oligo-2 |
| | PEG-DSPE: | | |
| | C16mPEG-Ceramide | | |
| NP6 | Compd 6: DOPE: Chol: | 18:60:20:1:1 | Oligo-3 |
| | PEG-DSPE: | | |
| | C16mPEG-Ceramide | | |
| NP7 | Compd 6: DOPE: Chol: | 18:60:20:1:1 | none |
| | PEG-DSPE: | | |
| | C16mPEG-Ceramide | | |

### Example 21

### In Vitro Efficacy of Nanoparticles on mRNA Down-Regulation in a Variety of Human Cancer Cells: Gastric Cancer, Lung Cancer, Prostate Cancer, Breast Cancer and KB Cancer

The efficacy of the nanoparticles described herein is evaluated in a variety of cancer cells, for example, human gastric cancer cells (N87cell line), human lung cancer cells (A549 cell line), human prostate cancer cells (15PC3 cell line or DU145 cell line), human breast cancer cells (MCF7 cell line), human KB cancer cells (KB cell line). The cells are treated with one of the following: nanoparticles encapsulating antisense ErbB3 oligonucleotides (Sample NP5), nanoparticles encapsulating oligonucleotides with a scrambled sequence (Sample No. NP6) or empty placebo nanoparticles (Sample No. NP7). The in vitro efficacy of each of the nanoparticles on downregulation of ErbB3 expression is measured by the procedures described in Example 2.

### Example 22

### In Vivo Efficacy of Nanoparticles on mRNA Down-Regulation in Tumor and Liver of Human Prostate Cancer Xenografted Mice Model

The in vivo efficacy of nanoparticles described herein is evaluated in human prostate cancer xenografted mice. The 15PC3 human prostate tumors are established in nude mice by subcutaneous injection of 5×10⁶ cells/mouse into the right auxiliary flank. When tumors reach the average volume of 100 mm³, the mice are randomly grouped 5 mice per group. The mice of each group are treated with nanoparticle encapsulating antisense ErbB3 oligonucleotides (Sample NP5) or corresponding naked oligonucleotides (Oligo 2). The nanoparticles are given intravenously (i.v.) at 15 mg/kg/dose, 5 mg/kg/dose, 1 mg/kg/dose, or 0.5 mg/kg/dose at q3d x 4 for 12 days. The dosage amount is based on the amount of oligonucleotides in the nanoparticles. The naked oligonucleotides are given intraperitoneally (i.p.) at 30 mg/kg/dose or intravenously at 25 mg/kg/dose or 45 mg/kg/dose at q3d x 4 for 12 days. The mice are sacrificed twenty four hours after the final dose. Plasma samples are collected from the mice and stored at −20° C. Tumor and liver samples are also collected from the mice. The samples are analyzed for mRNA KD in the tumors and livers.

### Example 23

### In Vivo Efficacy of Nanoparticles on mRNA Down-Regulation in Human Colon Cancer Xenografted Mice Model

The in vivo efficacy of the nanoparticles described hrein is evaluated in human colon cancer xenografted mice. The nanoparticles described herein (Sample NP5) are given via intratumoral injection to the mice with human DLD-1 tumors at q3dx4 for 12 days. The naked oligonucleotides (Oligo 2), scrambled oligonucloetides (Oligo 3), and nanoparticles containing scrambled oligonucleotides (Sample NP6) are also given to the mice. Tumor samples from the mice of each test group are collected and analyzed by using qRT-PCR for mRNA down-regulation.

### Example 24

### In Vivo Efficacy of Nanoparticles on mRNA Down-Regulation in Human Cancer Xenografted Mice Model with Metastatis in Liver

The in vivo efficacy of the nanoparticles described herein is evaluated in human cancer xenografted mice with metastasis to the liver. The A549 cancer cells are injected intrasplenically, followed by a splenectomy to establish metastatic liver disease. Two days following the splenectomy, the mice of each group are intravenously given nanoparticles encapsulating antisense ErbB3 oligonucleotides (Sample NP5) or scrambled oligonucleotides (Sample NP6) at 0.5 mg/kg/dose at q3d x 10. Naked antisense ErbB3 oligonucleotides (Oligo 2) are given intravenously at 35 mg/kg/dose at q3d x 4. The survival of the animals is observed.

As used herein the terms “including,” “containing” and “comprising” and alternate word forms thereof do not exclude the possibility of further constituents being present. Wherever the terms “including,” “containing” and “comprising” and alternate word forms thereof are used in this disclosure in the description of various embodiments of the invention, it should be understood that this disclosure is also teaching corresponding embodiments in which one or more of said terms is limited to mean “consisting essentially of” or “consisting of” or the like.

1. A cationic lipid of Formula (I): 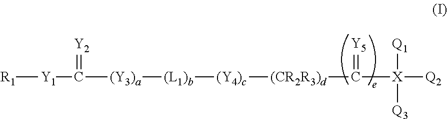
wherein
R₁ is a cholesterol or analog thereof;
Y₁, Y₂ and Y₅ are independently O, S or NR₄;
Y₃ and Y₄ are independently O, S or NR₅;
L₁ is a spacer having a substituted saturated or unsaturated, branched or linear, C₃₋₅₀ alkyl, wherein one or more carbons are replaced with NR₆, O, S or C(═Y), wherein Y is O, S or NR₄;
(a), (c) and (e) are independently 0 or 1;
(b) is 0 or a positive integer, provided that when (b) is 0, both (a) and (c) are not simultaneously positive integers;
(d) is 0 or a positive integer;
X is C or P;
Q₁ is H, C₁₋₆ alkyl, NH₂, or -(L₁₁)_{d1}-R₁₁;
Q₂ is H, C₁₋₆ alkyl, NH₂, or -(L₁₂)_{d2}-R₁₂;
Q₃ is (═O), 14, C₁₋₆ alkyl, NH₂, or -(L₁₃)_{d3}-R₁₃,
provided that
(i) when X is C, Q₃ is not (═O); and
(ii) when X is P, (e) is 0,
wherein
L₁₁, L₁₂ and L₁₃ are independently selected bifunctional spacers;
(d1), (d2) and (d3) are independently 0 or a positive integer;
R₁₁, R₁₂ and R₁₃ are independently hydrogen, NH₂, 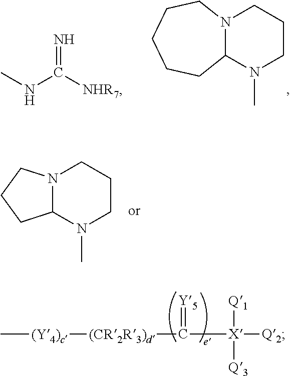
wherein
Y′₄ is O, S, or NR′₅;
Y′₅ are independently O, S or NR′₄;
(c′) and (e′) are independently 0 or 1;
(d′) is 0 or a positive integer;
X′ is C or P;
Q′₁ is H, C₁₋₆ alkyl, NH₂, or -(L′₁₁)_{d′1}-R′₁₁;
Q′₂ is H, C₁₋₆ alkyl, NH₂, or -(L′₁₂)_{d′2}-R′₁₂;
Q′₃ is (═O), H, C₁₋₆ alkyl, NH₂, or -(L′₁₃)_{d′3}-R′₁₃,
provided that
(i) when X′ is C, Q′₃ is not (═O); and
(ii) when X′ is P, (e′) is 0,
wherein
L′₁₁, L′₁₂ and L′₁₃ are independently selected bifunctional spacers;
(d′1), (d′2) and (d′3) are independently 0 or a positive integer;
R′₁₁, R′₁₂ and R′₁₃ are independently hydrogen, NH₂, 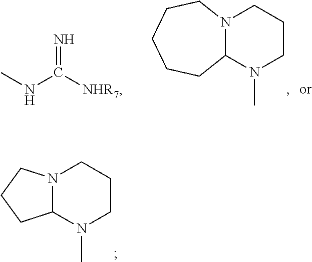
and
R₂₋₇, R′₂₋₅ and R′₇ are independently selected from among hydrogen, amino, substituted amino, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, and substituted C₁₋₆ heteroalkyl,
provided that at least one of Q₁₋₃ and Q′₁₋₃ includes

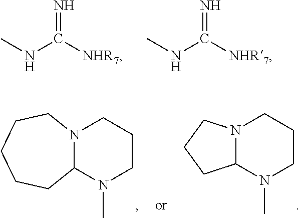

2-5. (canceled)

6. The cationic lipid of claim 1, wherein L₁, when combined with a moiety of (Y₄)_{c}—(CR₂R₃)_{d}—C(═Y₅)ₑ, is independently selected from the group consisting of:
—(CR₂₁R₂₂)ₜ₁—[C(═Y₆)ₑ₁—(Y₄)_{c}—(CR₂R₃)_{d}—C(═Y₅)ₑ—;
—(CR₂₁R₂₂)ₜ₁Y₇—(CR₂₃R₂₄)ₜ₂—(Y₈)ₑ₂—[C(═Y₆)]ₑ₁—(Y₄)_{c}—(CR₂R₃)_{d}—C(═Y₅)ₑ—,
—(CR₂₁R₂₂CR₂₃R₂₄Y₇)ₜ₃—[C(═Y₆)]ₑ₁—(Y₄)_{c}—(CR₂R₃)_{d}—C(═Y₅)ₑ—,
—(CR₂₁R₂₂CR₂₃R₂₄Y₇)ₜ₃(CR₂₅R₂₆)ₜ₄—(Y₈)ₑ₂—[C(═Y₆)]ₑ₁—(Y₄)_{c}—(CR₂R₃)_{d}—C(═Y₅)ₑ—,
—(CR₂₁R₂₂CR₂₃R₂₄Y₇)ₜ₃(CR₂₅R₂₆)ₜ₄—(Y₈)ₑ₂—[C(═Y₆)]ₑ₁—(CR₂₇R₂₈)ₜ₁—(Y₄)_{c}—(CR₂R₃)_{d}—C(═Y₅)ₑ—,
—[(CR₂₁R₂₂CR₂₃R₂₄)ₜ₅Y₇]ₜ₆(CR₂₅R₂₆)ₜ₄—(Y₈)ₑ₂—[C(═Y₆)]ₑ₁—(Y₄)_{c}—(CR₂R₃)_{d}—C(═Y₅)ₑ—,
—(CR₂₁R₂₂)ₜ₁—[(CR₂₃R₂₄)ₜ₂Y₇]ₜ₇(CR₂₅R₂₆)ₜ₄—(Y₄)_{c}—(CR₂R₃)_{d}—C(═Y₅)ₑ—,
—(CR₂₁R₂₂)ₜ₁—[(CR₂₃R₂₄)ₜ₂Y₇]ₜ₇(CR₂₅R₂₆)ₜ₄—(Y₈)ₑ₂—[C(═Y₆)]ₑ₁—(Y₄)_{c}—(CR₂R₃)_{d}—C(═Y₅)ₑ—,
—(CH₂)₄—C(═O)—,
—(CH₂)₅—C(═O)—,
—(CH₂)₆—C(═O)—,
—CH₂CH₂O—CH₂O—C(═O)—,
—(CH₂CH₂O)₂—CH₂O—C(═O)—,
—(CH₂CH₂O)₃—CH₂O—C(═O)—,
—(CH₂CH₂O)₂—C(═O)—,
—CH₂CH₂O—CH₂CH₂NH—C(═O)—,
—(CH₂CH₂O)₂—CH₂CH₂NH—C(═O)—,
—(CH₂CH₂O)₂—CH₂CH₂NH—C(═O)—CH₂NHC(═O)—,
—(CH₂CH₂O)₂—CH₂CH₂O—C(═O)—,
—CH₂—O—CH₂CH₂O—CH₂CH₂NH—C(═O)—,
—CH₂—O—(CH₂CH₂O)₂—CH₂CH₂NH—C(═O)—,
—CH₂—O—CH₂CH₂O—CH₂C(═O)—,
—CH₂—O—(CH₂CH₂O)₂—CH₂C(═O)—,
—(CH₂)₄—C(═O)NH—,
—(CH₂)₅—C(═O)NH—,
—(CH₂)₆—C(═O)NH—,
—CH₂CH₂O—CH₂O—C(═O)—NH—,
—(CH₂CH₂O)₂—CH₂O—C(═O)—NH—,
—(CH₂CH₂O)₃—CH₂O—C(═O)—NH—,
—(CH₂CH₂O)₂—C(═O)—NH—,
—(CH₂CH₂O)₂—CH₂C(═O)—NH—,
—CH₂CH₂O—CH₂CH₂NH—C(═O)—NH—,
—(CH₂CH₂O)₂—CH₂CH₂NH—C(═O)—NH—,
—CH₂—O—CH₂CH₂O—CH₂CH₂NH—C(═O)—NH—,
—CH₂—O—(CH₂CH₂O)₂—CH₂CH₂NH—C(═O)—NH—,
—CH₂—O—CH₂CH₂O—CH₂C(═O)—NH—,
—CH₂—O—(CH₂CH₂O)₂—CH₂C(═O)—NH—,
—(CH₂CH₂O)₂—,
—(CH₂CH₂O)₃—,
—CH₂CH₂O—CH₂O—,
—(CH₂CH₂O)₂—CH₂CH₂NH—,
—(CH₂CH₂O)₃—CH₂CH₂NH—,
—CH₂CH₂O—CH₂CH₂NH—,
—(CH₂CH₂O)₂—CH₂CH₂NH—,
—CH₂—O—CH₂CH₂O—CH₂CH₂NH—,
—CH₂—O—(CH₂CH₂O)₂—CH₂CH₂NH—,
—CH₂—O—CH₂CH₂O— and
—CH₂—O—(CH₂CH₂O)₂—
wherein:
Y₆ is O, NR₂₉, or S;
Y₇₋₈ are independently O, NR₂₉, or S;
R₂₁₋₂₉ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
each of (t1), (t2), (t3), (t4), (t5), (t6) and (t7) is independently zero or a positive integer;
each (c), (e), (e1) and (e2) are independently zero or 1; and
all the other variables are as defined above.

7. (canceled)

8. The cationic lipid of claim 1, wherein L₁₁₋₁₃ and L′₁₁₋₁₃ are independently selected from the group consisting of:
—(CR′₂₁R′₂₂)_{q1}(Y′₈)_{v′}[C(═Y′₉)]ᵥ(CR′₂₃R′₂₄)_{q2}—,
—(CR′₂₁R′₂₂)_{q1}(Y′₈)_{v′}[C(═Y′₉)]ᵥY′₁₀(CR′₂₃R′₂₄)_{q2}—,
—(CR′₂₁R′₂₂)_{q1}(Y′₈)_{v′}[C(═Y′₉)]ᵥ(CR′₂₃R′₂₄)_{q2}—Y′₁₁—(CR′₂₃R′₂₄)_{q3}—,
—(CR′₂₁R′₂₂)_{q1}(Y′₈)_{v′}[C(═Y′₉)]ᵥY′₁₀(CR′₂₃R′₂₄)_{q2}—Y′₁₁—(CR′₂₃R′₂₄)_{q3}—,
—(CR′₂₁R′₂₂)_{q1}(Y′₈)_{v′}[C(═Y′₉)]ᵥ(CR′₂₃R′₂₄CR′₂₅R′₂₆Y′₁₂)_{q4}(CR′₂₇CR′₂₈)_{q5}—,
—(CR′₂₁R′₂₂)_{q1}(Y′₈)_{v′}[C(═Y′₉)]ᵥY′₁₀(CR′₂₃R′₂₄CR′₂₅R′₂₆Y′₁₂)_{q4}(CR′₂₇CR′₂₈)_{q5}—, 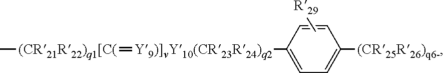
—(CH₂)₄—,
—(CH₂)₃—,
—O(CH₂)₂—
—C(═O)O(CH₂)₃—,
—C(═O)NH(CH₂)₃—,
—C(═O)(CH₂)₂—,
—C(═O)(CH₂)₃—,
—CH₂—C(═O)—O(CH₂)₃—,
—CH₂—C(═O)—NH(CH₂)₃—,
—CH₂—OC(═O)—O(CH₂)₃—,
—CH₂—OC(═O)—NH(CH₂)₃—,
—(CH₂)₂—C(═O)—O(CH₂)₃—,
—(CH₂)₂—C(═O)—NH(CH₂)₃—,
—CH₂C(═O)O(CH₂)₂—O—(CH₂)₂—,
—CH₂C(═O)NH(CH₂)₂—O—(CH₂)₂—,
—(CH₂)₂C(═O)O(CH₂)₂—O—(CH₂)₂—,
—(CH₂)₂C(═O)NH(CH₂)₂—O—(CH₂)₂—,
—CH₂C(═O)O(CH₂CH₂O)₂CH₂CH₂— and
—(CH₂)₂C(═O)O(CH₂CH₂O)₂CH₂CH₂—,
wherein:
Y′₈ and Y′₁₀₋₁₂ are independently O, NR′₃₀, or S;
Y′₉ are independently O, NR′₃₁, or S;
R^{′}₂₁₋₃₁ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
(q1), (q2), (q3), (q4), (q5), and (q6) are independently zero or a positive integer of from about 1 to about 10; and
(v) and (v′) are independently zero or 1.

9. (canceled)

10. The compound of claim 1, wherein the X(Q₁)(Q₂)(Q₃) moiety is

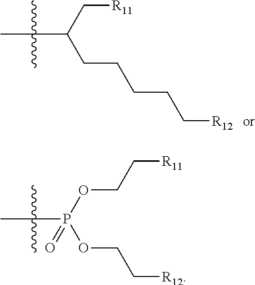

11. The cationic lipid of claim 1, having a Formula (Ia): 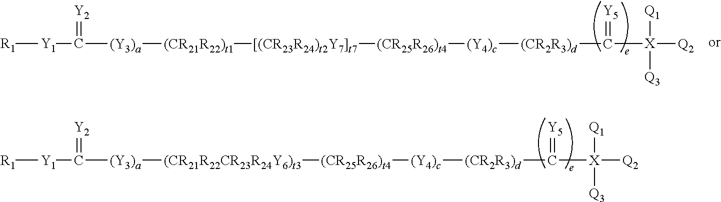
wherein
Y₆ and Y₇ are independently O, S or NR₂₉, preferably O or NH;
R₂₁₋₂₆ and R₂₉ are independently selected from among hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
(t1), (t2), (t3), (t4), and (t7) are independently 0 or a positive integer,
wherein R₂₁ and R₂₂ in each occurrence are independently the same or different, when (t1) is equal to or greater than 2;
wherein R₂₃, R₂₄, and Y₇ in each occurrence are independently the same or different, when (t2) and (t7) are indenpendently equal to or greater than 2,
wherein R₂₁, R₂₂, R₂₃, R₂₄, and Y₆, in each occurrence, are independently the same or different, when (t3) is equal to or greater than 2,
wherein R₂₅ and R₂₆ in each occurrence are independently the same or different,
when (t4) is equal to or greater than 2; and
all the other variables are as defined above.

12. The cationic lipid of claim 1 selected from the group consisting of:

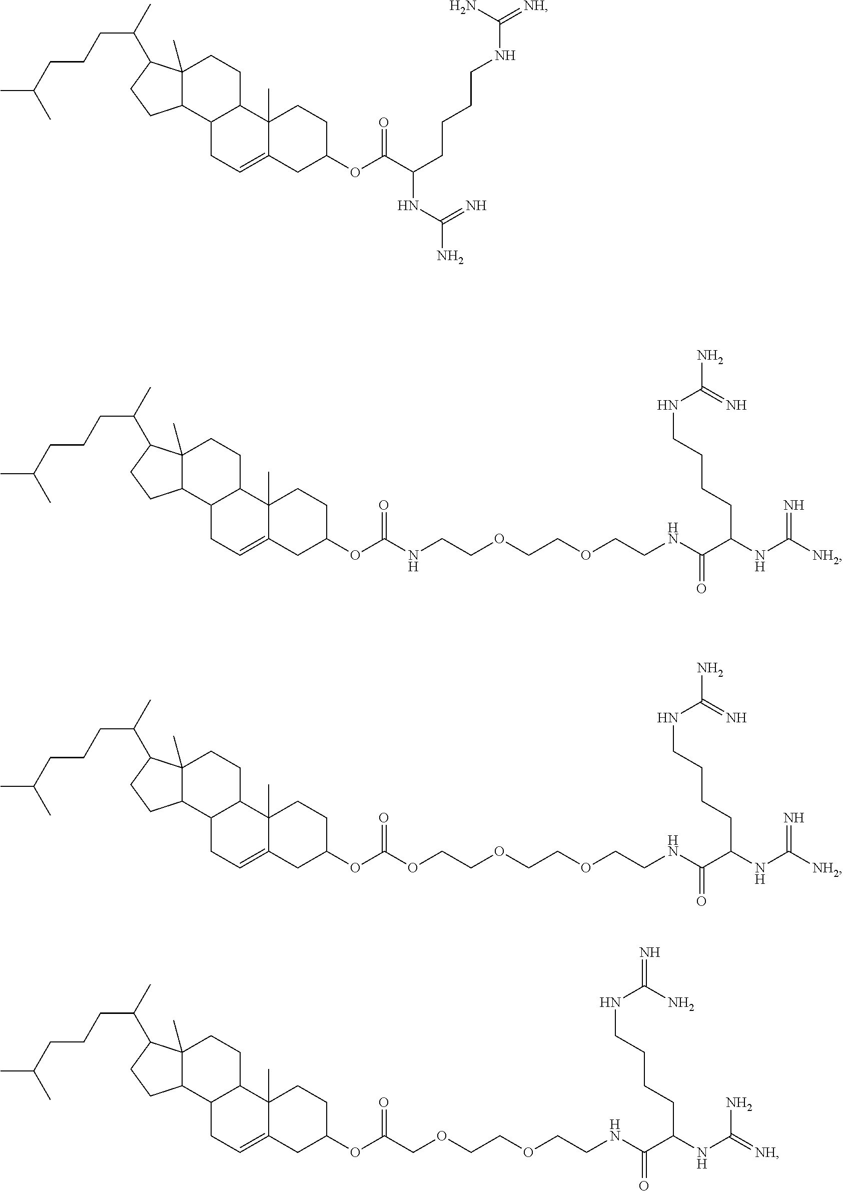

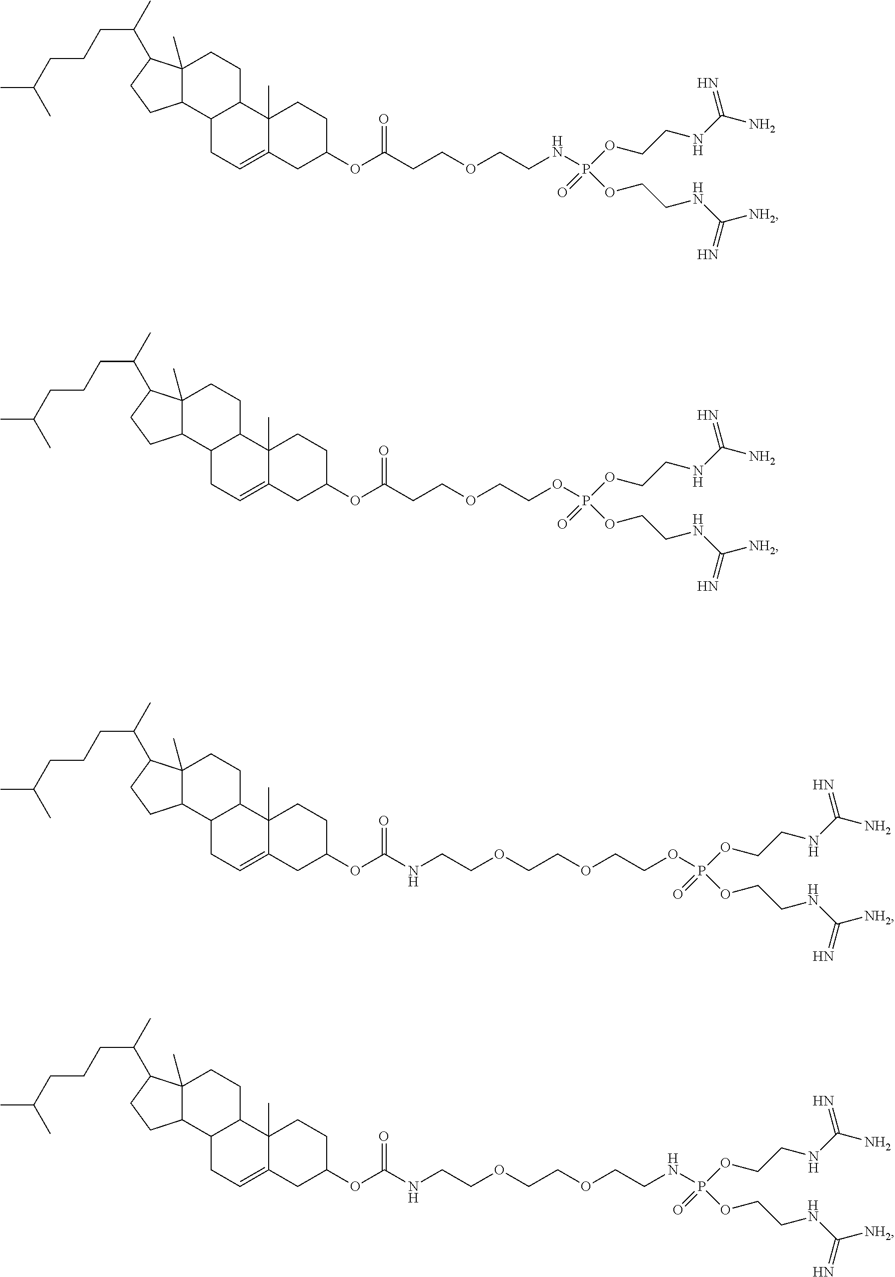

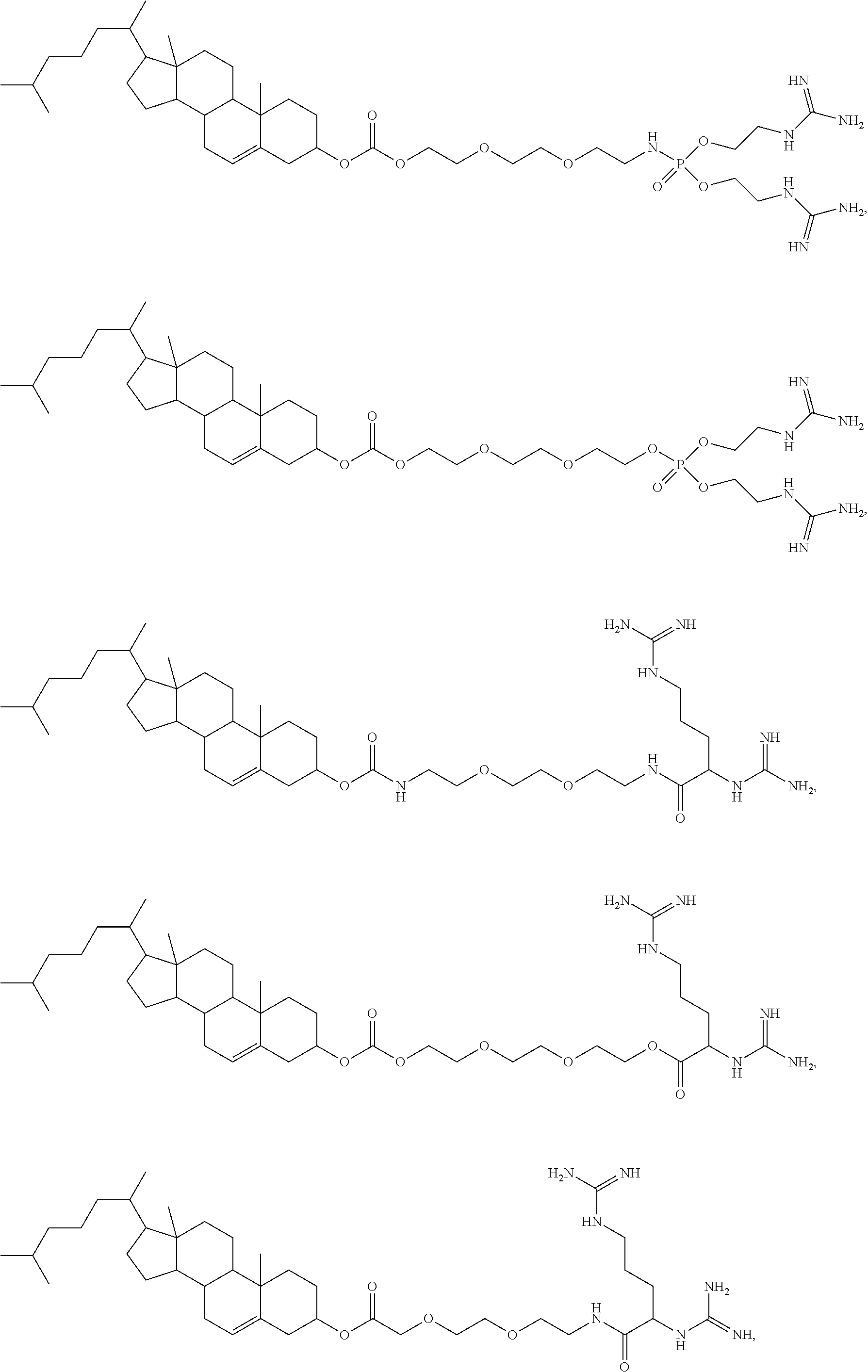

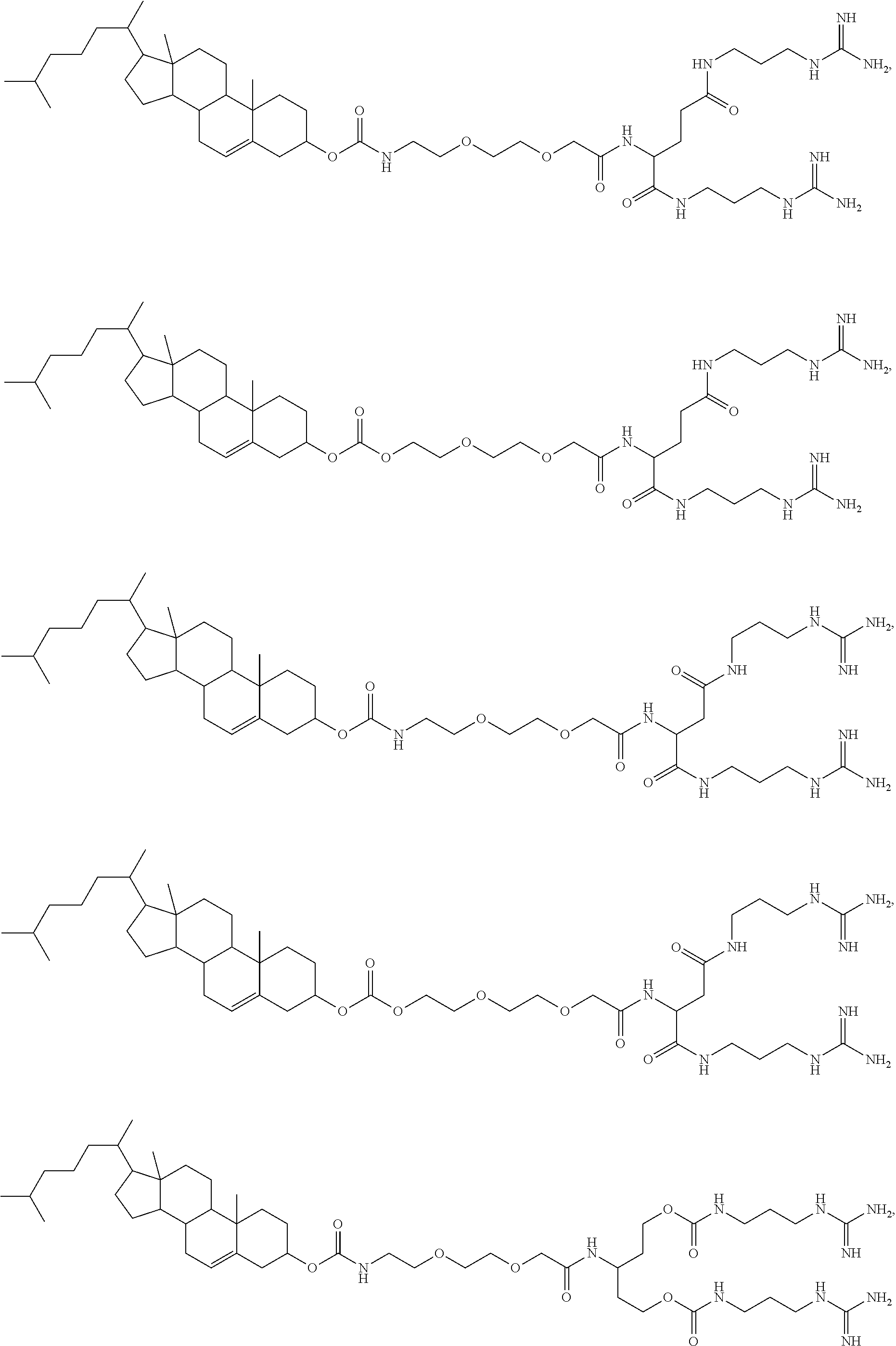

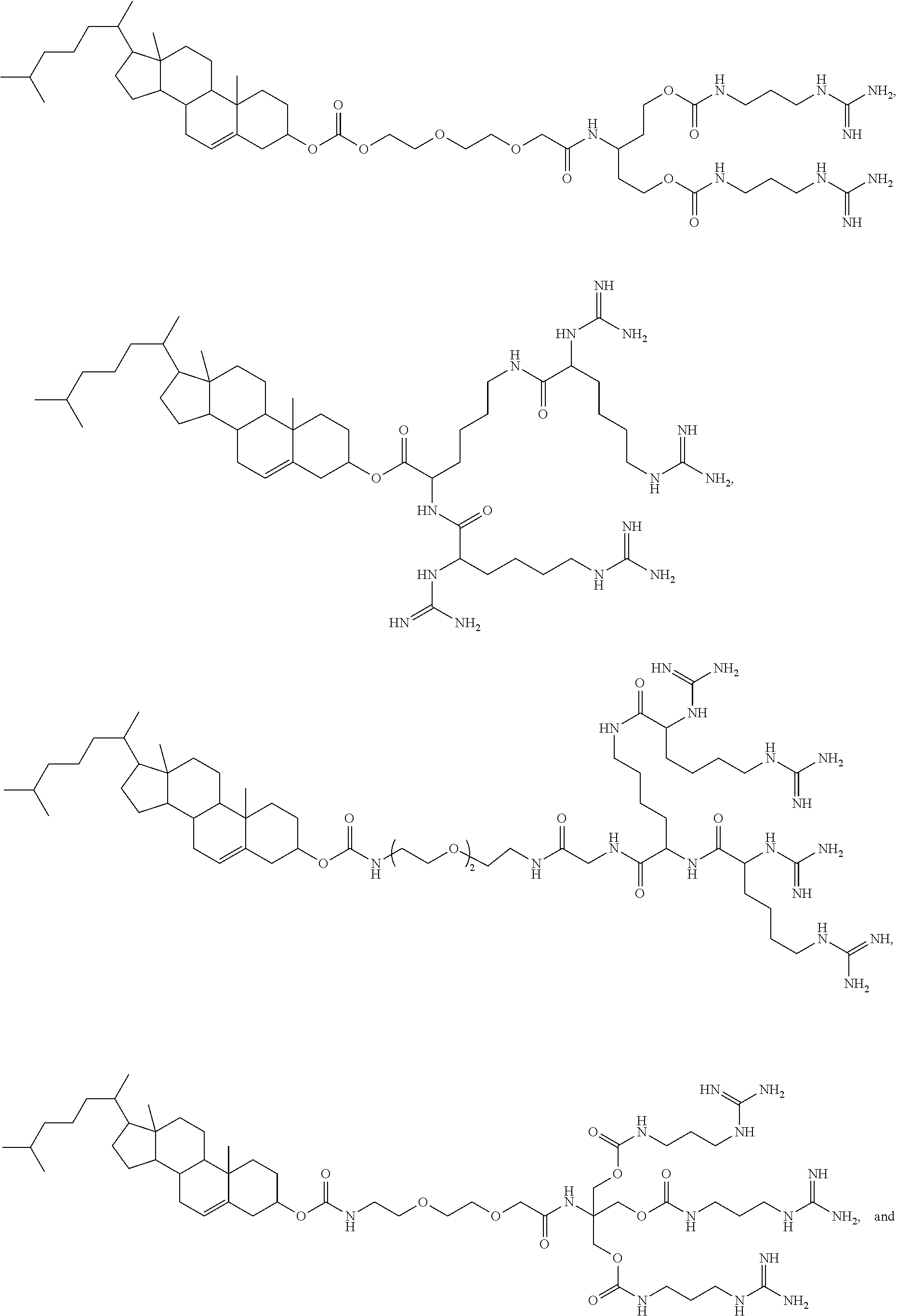

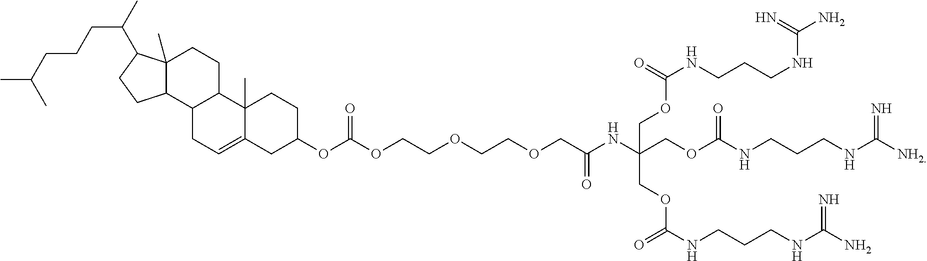

13. A nanoparticle composition comprising a cationic lipid of Formula (I) of claim 1.

14. The nanoparticle composition of claim 13, further comprising a fusogenic lipid and a PEG lipid.

15. The nanoparticle composition of claim 14, wherein the cationic lipid is selected from the group consisting of:

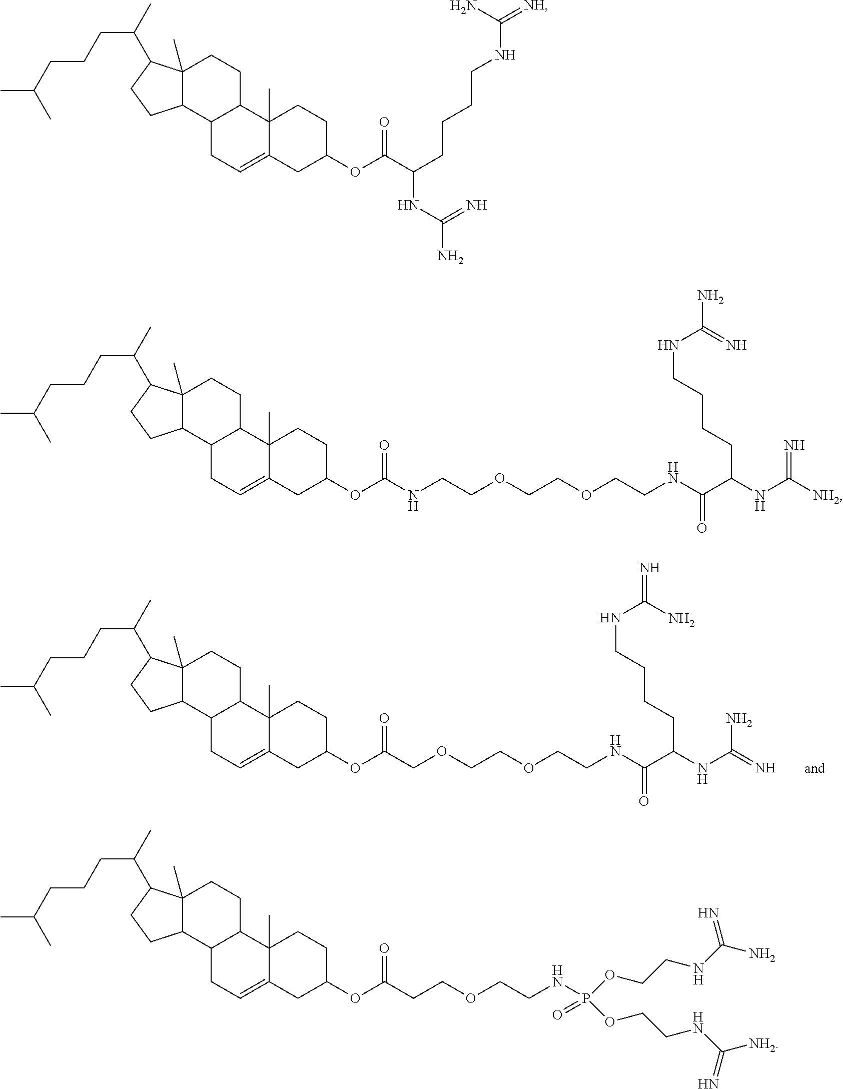

16. The nanoparticle composition of claim 14, wherein the fusogenic lipid is selected from the group consisting of DOPE, DOGP, POPC, DSPC, EPC and combinations thereof, and wherein the PEG lipid is selected from the group consisting of PEG-DSPE, PEG-dipalmitoylglycamide, C16mPEG-ceramide and combinations thereof.

17. (canceled)

18. The nanoparticle composition of claim 14, further comprising cholesterol.

19. The nanoparticle composition of claim 14 selected from the group of a mixture of:
a cationic lipid of Formula (I), a diacylphosphatidylethanolamine, a PEG conjugated to phosphatidylethanolamine (PEG-PE), and cholesterol;
a cationic lipid of Formula (I), a diacylphosphatidylcholine, a PEG conjugated to phosphatidylethanolamine (PEG-PE), and cholesterol;
a cationic lipid of Formula (I), a diacylphosphatidylethanolamine, a diacylphosphatidyl-choline, a PEG conjugated to phosphatidylethanolamine (PEG-PE), and cholesterol;
a cationic lipid of Formula (I), a diacylphosphatidylethanolamine, a PEG conjugated to ceramide (PEG-Cer), and cholesterol; and
a cationic lipid of Formula (I), a diacylphosphatidylethanolamine, a PEG conjugated to phosphatidylethanolamine (PEG-PE), a PEG conjugated to ceramide (PEG-Cer), and cholesterol.

20. The nanoparticle composition of claim 18, wherein the cationic lipid has a molar ratio ranging from about 10% to about 99.9% of the total lipid present in the nanoparticle composition.

21. (canceled)

22. The nanoparticle composition of claim 18, wherein a molar ratio of a cationic lipid, a non-cholesterol-based fusogenic lipid, a PEG lipid and cholesterol is about 15-25%:20-78%:0-50%:2-10%: of the total lipid present in the nanoparticle composition.

23. The nanoparticle composition of claim 18, wherein the cationic lipid, DOPE, cholesterol, and C16mPEG-Ceramide is included in a molar ratio of about 17%:60%:20%:3% of the total lipid present in the nanoparticle composition, wherein the cationic lipid is

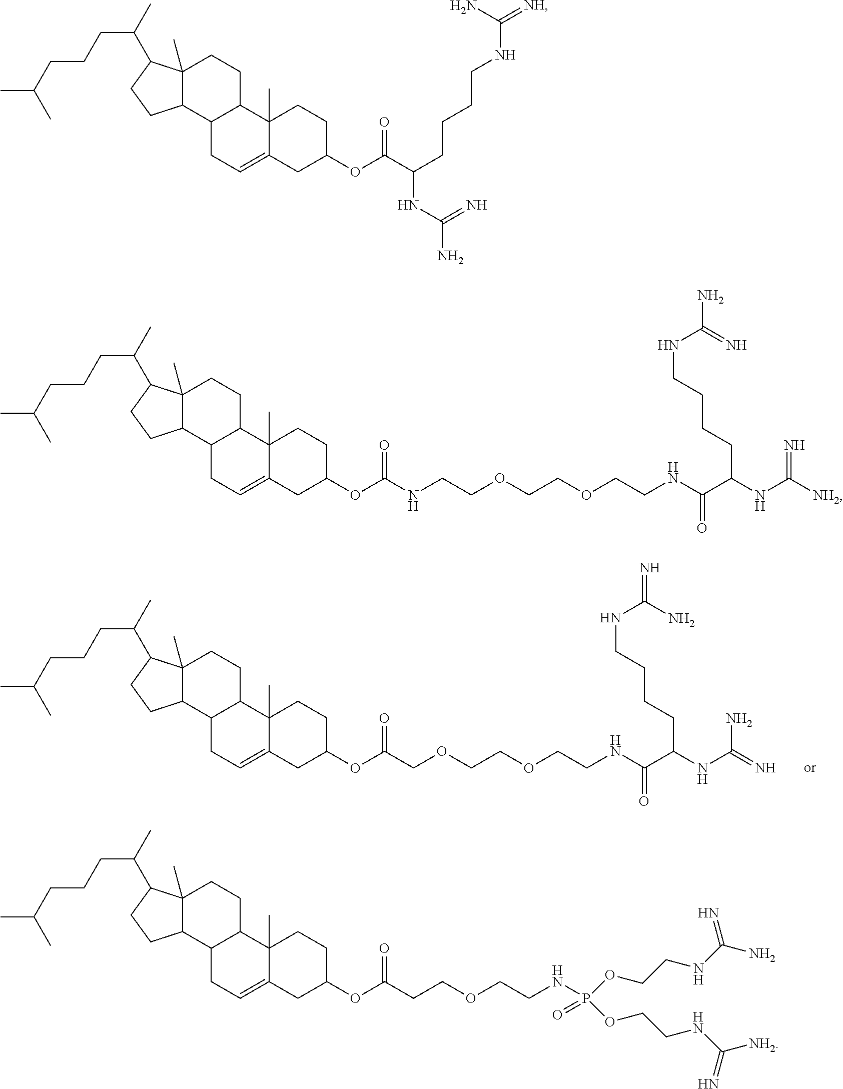

24. The nanoparticle composition of claim 18 comprising nucleic acids encapsulated with the nanoparticle composition.

25. The nanoparticle of claim 24, wherein the nucleic acids is a single stranded or double stranded oligonucleotide.

26. The nanoparticle of claim 24, wherein the nucleic acids is selected from the group consisting of deoxynucleotide, ribonucleotide, locked nucleic acids (LNA), short interfering RNA (siRNA), microRNA (miRNA), aptamers, peptide nucleic acid (PNA), phosphorodiamidate morpholino oligonucleotides (PMO), tricyclo-DNA, double stranded oligonucleotide (decoy ODN), catalytic RNA (RNAi), aptamers, spiegelmers, CpG oligomers and combinations thereof.

27-30. (canceled)

31. The nanoparticle of claim 25, wherein the oligonucleotide inhibits expression of oncogenes, pro-angiogenesis pathway genes, pro-cell proliferation pathway genes, viral infectious agent genes, and pro-inflammatory pathway genes.

32. The nanoparticle of claim 25, wherein the oligonucleotide is selected from the group consisting of antisense HIF-1α oligonucleotides, antisense survivin oligonucleotides, antisense ErbB3 oligonucleotides, β-catenin oligonucleotides and antisense Bcl-2 oligonucleotides.

33. The compound of claim 25, wherein the oligonucleotide comprises eight or more consecutive nucleotides set forth in SEQ ID NO: 1, SEQ ID NOs 2 and 3, SEQ ID NO: 3, SEQ ID NO: 4, SEQ ID NO: 5, SEQ ID NO: 5, and SEQ ID NO: 6.

34. The nanoparticle of claim 24, wherein the charge ratio of the cationic lipid and the nucleic acids ranges from about 1:1 to about 20:1.

35. The nanoparticle of claim 24, wherein the nanoparticle has a size ranging from about 50 nm to about 150 nm.

36-37. (canceled)

38. A method of inhibiting or downregulating a gene expression in human cells or tissues, comprising:
contacting human cells or tissues with a nanoparticle of claim 24.

39-40. (canceled)

41. A method of inhibiting the growth or proliferation of cancer cells comprising:
contacting a cancer cell with a nanoparticle of claim 24.

42. The method of claim 41, further comprising administering a chemotherapeutic agent.

43. A method of treating a cancer in a mammal, comprising:
administering an effective amount of a nanoparticle of claim 15 to a mammal in need thereof.

44. The method of claim 43, wherein the cancer is metastatic into the liver.
